(19) United States
(12) Patent Application Publication
(10) Pub. No.: US 2026/0207748 A1
(43) Pub. Date: Jul. 23, 2026

(54) ENGINEERED T CELL RECEPTORS AND ENGINEERED IMMUNE CELLS EXPRESSING THE SAME

(71) Applicants: TCRCURE BIOPHARMA CORP., Los Angeles, CA (US); GUANGDONG TCRCURE BIOPHARMA TECHNOLOGY CO., LTD., Guangzhou, Guangdong (CN); TCRCURE HONG KONG LTD., Hong Kong (CN)

(72) Inventors: Si Li, Alhambra, CA (US); Weiting Du, Arcadia, CA (US); Hongbo Yang, Los Angeles, CA (US); Lixia Zhao, Cary, NC (US); Xiaowei Zhang, Durham, NC (US); Zhenbo Su, Los Angeles, CA (US)

(21) Appl. No.: 19/142,471

(22) PCT Filed: Dec. 22, 2023
(86) PCT No.: PCT/US2023/085790

§ 371 Date: Jun. 23, 2025

Related U.S. Application Data
(60) Provisional application No. 63/477,071, filed on Dec. 23, 2022.
(60) Provisional application No. 63/613,545, filed on Dec. 21, 2023.

(51) Int. Cl. A61K 40/42; A61K 40/11; A61K 40/15; A61K 40/31; C07K 14/54; C07K 14/705; C07K 16/40; C12N 5/0783

(52) U.S. Cl. CPC A61K 40/4252; A61K 40/11; A61K 40/15; A61K 40/31; C07K 14/5437; C07K 14/705; C07K 14/7051; C07K 14/70521; C07K 14/70578; C07K 16/40; C12N 5/0636; C07K 2317/622; C07K 2319/00; C12N 2510/00

(57) ABSTRACT

Engineered T cell receptors (TCRs) are provided, including an engineered CD3 zeta subunit and/or target-recognizing TCR subunit. The engineered CD3 zeta subunit includes a co-stimulatory region operably linked to or incorporated into an intracellular domain of a mammalian CD3 zeta component. The engineered target-recognizing TCR subunit includes an antigen-binding region or a ligand as the target-recognizing moiety that is operably linked to or incorporated into an extracellular domain of any of mammalian TCRα, TCRβ, CD3ε, CD3γ, or CD3δ subunits or a functional portion or variant thereof. Some embodiments of the engineered target-recognizing TCR subunit include a truncated CD3 epsilon subunit lacking the intracellular domain thereof, and optionally further includes at least one functional region. This disclosure also provides immune cells expressing any of the engineered TCR subunit, some of which exhibit improved characteristics including more specific and efficient cytotoxicity against target cells, as well as favorably safer therapeutic profiles.

### CLAIM OF PRIORITY

This application claims the benefit of U.S. Provisional Application No. 63/477,071, filed on Dec. 23, 2022, and U.S. Provisional Application No. 63/613,545, filed on Dec. 21, 2023. The entire contents of the foregoing applications are incorporated herein by reference in their entirety.

### TECHNICAL FIELD

This disclosure relates to engineered T cell receptors (TCRs) and engineered immune cells expressing the same. In certain embodiments, the engineered T cell receptors include an engineered CD3 zeta subunit and/or an engineered CD3 epsilon subunit. In certain embodiments, the engineered T cell receptors include an engineered target-recognizing T cell receptor (TCR) subunit where the target-recognizing moiety is a ligand moiety.

### BACKGROUND

T cell receptor (TCR) is a protein complex that is expressed on the surface of T lymphocytes, which is generally responsible for recognizing fragments of antigens bound to major histocompatibility complex (MHC).

A T cell receptor typically comprises a membrane-anchored heterodimer (i.e. TCR alpha (TCRα or TCRa) and beta (TCRβ or TCRb) chains), each composed of a Variable (V) region, a Constant (C) region, a transmembrane region and a short cytoplasmic tail, with the Variable region containing hypervariable sequences and being responsible for the specific biding to the antigen/MHC complex. Yet TCR heterodimer itself is incapable of transducing an activation signal, which relies on a CD3 signal-transduction complex that is complexed with TCR heterodimer. The CD3 complex consists of 1 CD3 gamma (CD3γ or CD3g) chain, 1 CD3 delta (CD3δ or CD3d) chain, 2 CD3 epsilon (CD3ε or CD3e) chains, and 2 CD3 zeta (CD3ζ or CD3z) chains, which together allow the activation signals to be transduced to the interior of the T cells upon the interaction of antigen with the TCR heterodimer. The structure of a complete TCR-CD3 complex (i.e. “TCR signaling complex”, or “TCR complex”) is illustrated in FIG. 1. The various TCR heterodimer subunit (TCRα or TCRβ) and the various CD3 subunit (i.e. CD3γ, CD3δ, CD3ε and CD3ζ subunit) can be regarded as a TCR signaling complex subunit (also exchangeable to “TCR subunit”). A detailed description of TCR, and the use its functions are described, e.g., in Alcover, A. Et al. “Cell biology of T cell receptor expression and regulation.” Annual Review of Immunology 36 (2018): 103-125; Gaud, G. et al. “Regulatory mechanisms in T cell receptor signalling.” Nature Reviews Immunology 18.8 (2018): 485-497; and Shah, K., et al. “T cell receptor (TCR) signaling in health and disease.” Signal Transduction and Targeted Therapy 6.1 (2021): 412; each of which is incorporated by reference in its entirety.

TCR signaling plays an important role in medicine, including the treatment of cancer. However, in some cases, TCR signaling can be insufficient to activate T cells. Thus, there is a need in the art for engineered TCRs with improved activities.

### SUMMARY

The present disclosure provides an engineered T cell receptor (TCR) complex termed Antigen-specificity redirected TCR complex (or “Aspire-TCR”), which substantially comprises an engineered target-recognizing subunit and/or an exogenous CD3 zeta (i.e. CD3ζ or CD3z) subunit. Such engineered TCR complex is capable of specifically and efficiently recognizing specific target molecules expressed in target cells (e.g., tumor cells). When expressed in immune cells, the engineered T cell receptor (TCR) complex can confer the specific and efficient cytotoxicity of the immune cells expressing the engineered TCR complex against the target cells.

In a first aspect, the present disclosure provides an engineered CD3 zeta (CD3z) subunit, which comprises a co-stimulatory region that is operably linked to or incorporated into a CD3 zeta component. The CD3 zeta component comprises a mammalian CD3 zeta or a functional portion or a functional variant thereof, and the co-stimulatory region is within an intracellular domain of the engineered CD3 zeta subunit.

According to some embodiments, the CD3 zeta component comprises a human CD3 zeta or a functional portion or a functional variant thereof, comprising a sequence that has at least 80% sequence identity to SEQ ID NO: 1.

According to some embodiments, when the engineered CD3z subunit is expressed in an immune cell, at least one of the following is met: (1) the immune cell expressing the engineered CD3 zeta subunit has a reduced activation in the absence of antigen stimulation compared to when the immune cell does not express the engineered CD3 zeta subunit; (2) the immune cell expressing the engineered CD3 zeta subunit has a reduced cytotoxicity against non-target cells compared to when the immune cell does not express the engineered CD3 zeta subunit; (3) the immune cell expressing the engineered CD3 zeta subunit has an increased activation upon antigen stimulation compared to when the immune cell does not express the engineered CD3 zeta subunit; and (4) the immune cell expressing the engineered CD3 zeta subunit has an increased immune cell response against target cells corresponding thereto compared to when the immune cell does not express the engineered CD3 zeta subunit.

According to some embodiments, the CD3 zeta component is a full-length wildtype human CD3 zeta subunit comprising an amino acid sequence as set forth in SEQ ID NO: 1.

In any embodiments of the engineered CD3z subunit as described above, the co-stimulatory region may comprise a co-stimulatory domain, or a functional portion or a functional variant thereof, of CD28, 4-1BB, OX40, CD2, CD27, CDS, ICAM-1, LFA-1, or ICOS. As such, according to some embodiments of the engineered CD3 zeta subunit, the co-stimulatory region comprises a co-stimulatory domain, or a functional portion or a functional variant thereof, of CD28, comprising a sequence that has at least 80% sequence identity to SEQ ID NO: 2; yet according to some other embodiments of the engineered CD3 zeta subunit, the co-stimulatory region comprises a co-stimulatory domain, or a functional portion or a functional variant thereof, of 4-1BB, comprising a sequence that has at least 80% sequence identity to SEQ ID NO: 3.

According to some embodiments of the engineered CD3 zeta subunit, the co-stimulatory region is operably fused to the C-terminus of the CD3 zeta component. Herein the fusion between the co-stimulatory region and the CD3 zeta component may optionally be through a linker (such as a flexible linker) but may optionally be direct without any separator sequence or any linker therebetween. According to yet some other embodiments of the engineered CD3 zeta subunit, the co-stimulatory region is located between a transmembrane (TM) domain and an intracellular domain (ICD) of the CD3 zeta component (i.e. the co-stimulatory region is inserted at a region between TM and ICD of the CD3z subunit).

According to some embodiments, the engineered CD3 zeta subunit further comprises a STAT binding region within an intracellular domain thereof.

Herein in some embodiments, the STAT binding region may comprise a STAT3 binding motif comprising an amino acid sequence that is at least 50%, 75%, or 100% identical to the amino acid sequence of SEQ ID NO: 22, and further optionally, the STAT3 binding motif comprises an amino acid sequence that is at least 50%, 75%, or 100% identical to the amino acid sequence of SEQ ID NO: 21; in yet some other embodiments, the STAT binding region may comprise a STAT5 binding motif comprising an amino acid sequence that is at least 50%, 75%, or 100% identical to the amino acid sequence of SEQ ID NO: 23, and further optionally, the STAT5 binding motif comprises an amino acid sequence that is at least 50%, 75%, or 100% identical to the amino acid sequence of SEQ ID NO: 24.

Herein, the STAT binding region is optionally located within a region of the CD3 zeta component corresponding to positions 150-164 of SEQ ID NO: 1, and according to some embodiments, the STAT binding region is an insertion at a location of the CD3 zeta component corresponding to between position 157 and position 158 of SEQ ID NO: 1. As such, according to some embodiments where a STAT3 binding motif is inserted, the CD3 zeta component comprises an amino acid sequence that is at least 80%, 85%, 90%, 95%, or 100% identical to the amino acid sequence of SEQ ID NO: 20; yet according to some other embodiments where a STAT5 binding motif is inserted, the CD3 zeta component comprises an amino acid sequence that is at least 80%, 85%, 90%, 95%, or 100% identical to the amino acid sequence of SEQ ID NO: 29.

According to any of the above mentioned embodiments where the engineered CD3 zeta subunit comprises a STAT binding region within an intracellular domain thereof, when the engineered CD3 zeta subunit is expressed in a population of T cells, at least one of the following is met if compared to when the STAT binding region is absent in the engineered CD3 zeta subunit: (1) the population of T cells exhibit an increased expansion in vitro upon repeated stimulation with the antigen; (2) the population of T cells exhibit an increased efficacy in vivo against tumors that comprise the target cells; and (3) the population of T cells exhibit a higher ratio in tumor tissues infiltrated therewith in vivo.

In any of the embodiments of the engineered CD3 zeta subunit as described above, the immune cell may be a T lymphocyte (e.g. T cell), a tumor-infiltrating lymphocyte (TIL), or a natural kill (NK) cell. Herein, in some embodiments where the immune cell is a T cell, when the engineered CD3 zeta subunit is expressed in a population of T cells expressing a T cell receptor (TCR), the population of T cells exhibit an increased surface expression compared to when the co-stimulatory region is absent in the engineered CD3 zeta subunit.

Related to the above, a method for modulating activities of an immune cell is further provided. The method substantially comprises a step of expressing in the immune cell the engineered CD3 zeta subunit according to any one of the embodiments as described above. By means of the method as provided herein, at least one of the following is met for the immune cell expressing the engineered CD3 zeta subunit: (1) the immune cell has a reduced activation in the absence of antigen stimulation compared to when the immune cell does not express the engineered CD3 zeta subunit; (2) the immune cell has a reduced cytotoxicity against non-target cells compared to when the immune cell does not express the engineered CD3 zeta subunit; (3) the immune cell has an increased activation upon antigen stimulation compared to when the immune cell does not express the engineered CD3 zeta subunit; and (4) the immune cell has an increased immune cell response against target cells corresponding thereto compared to when the immune cell does not express the engineered CD3 zeta subunit.

In a second aspect, the present disclosure further provides an engineered CD3 epsilon (CD3e) subunit, which comprises a target-recognizing region (i.e. target-recognizing moiety or target-recognizing portion) and a CD3 epsilon component. The target-recognizing region is operably fused to an extracellular domain of the engineered CD3 epsilon subunit; and the CD3 epsilon component comprises a truncated CD3 epsilon with a truncation at a region of a mammalian CD3 epsilon subunit corresponding to the intercellular domain thereof.

Herein, according to some embodiments of the engineered CD3 epsilon subunit, the CD3 epsilon component comprises a truncated human CD3 epsilon. Optionally, the truncation is within a region of the human CD3 epsilon subunit corresponding to positions 151-179 of SEQ ID NO: 5, and as such, the CD3 epsilon component may optionally comprise an amino acid sequence that is at least 80%, 85%, 90%, 95%, or 100% identical to the amino acid sequence of SEQ ID NO: 10. Herein, it is further configured such that when the engineered CD3 epsilon subunit is expressed in a population of T cells, the population of T cells exhibit an increased surface expression of the engineered CD3 epsilon subunit and/or an increased expansion when compared to when the CD3 epsilon component comprises a full-length CD3 epsilon subunit.

According to some embodiments of the engineered CD3 epsilon subunit, the C-terminus of the CD3 epsilon component is the C-terminus of the engineered CD3 epsilon subunit. Herein, it is further configured such that when the engineered CD3 epsilon subunit is expressed in a population of T cells, at least one of the following is met: (1) the population of T cells exhibit an increased surface expression of the engineered CD3 epsilon subunit, comparing to when the CD3 epsilon component comprises a full-length CD3 epsilon subunit; (2) the population of T cells comprise a higher percentage of central memory T cells, comparing to when the CD3 epsilon component comprises a full-length CD3 epsilon subunit; and (3) the population of T cells exhibit an increased expansion in vitro upon repeated stimulation with the antigen, comparing to when the CD3 epsilon component comprises a full-length CD3 epsilon subunit.

According to some embodiments, the engineered CD3 epsilon subunit further comprises at least one functional region in an intracellular domain thereof, and the at least one functional region is operably fused to the C-terminus of the CD3 epsilon component.

Herein optionally, the at least one functional region comprises an immune receptor tyrosine based activation motif (ITAM), which may be derived from CD3z, CD3g, DAP12, FcαRI, FcγRI, FcγRII, FcγRIII, Dectin-1, CLEC-1, CD28, or CD72. According to some embodiments, the ITAM may comprise: (1) a CD3z ITAM3 that comprises an amino acid sequence that is at least 80%, 85%, 90%, 95%, or 100% identical to the amino acid sequence of SEQ ID NO: 11; or (2) a CD3z ITAM2-3 that comprises an amino acid sequence that is at least 80%, 85%, 90%, 95%, or 100% identical to the amino acid sequence of SEQ ID NO: 12, but may comprise other ITAM sequence(s) known to the field. According to some embodiments, the ITAM is located at, e.g., operably fused to, the C-terminus of the engineered CD3 epsilon subunit. Herein, the ITAM comprises a CD3z ITAM3 or a CD3z ITAM2-3. In some embodiments, the ITAM may further comprise a STAT binding region (e.g. a STAT3 binding motif or a STAT5 binding motif). Herein, it is further configured such that when the engineered CD3 epsilon subunit is expressed in a population of T cells, the population of T cells comprise a higher percentage of central memory T cells compared to when the ITAM is absent in the engineered CD3 epsilon subunit. It is to be noted that optionally the at least one functional region may comprise more than one ITAM, and each ITAM may have a same or a different sequence.

Optionally, the at least one functional region comprises any one or a combination of: (1) an FcεR1γ intracellular domain, which comprises an amino acid sequence that is at least 80%, 85%, 90%, 95%, or 100% identical to the amino acid sequence of SEQ ID NO: 25; (2) an OX40 intracellular domain, which comprises an amino acid sequence that is at least 80%, 85%, 90%, 95%, or 100% identical to the amino acid sequence of SEQ ID NO: 26; (3) a CD40 intracellular domain, which comprises an amino acid sequence that is at least 80%, 85%, 90%, 95%, or 100% identical to the amino acid sequence of SEQ ID NO: 27; (4) a DAP12 intracellular domain, which comprises an amino acid sequence that is at least 80%, 85%, 90%, 95%, or 100% identical to the amino acid sequence of SEQ ID NO: 28: (5) a 4-1BB intracellular domain, which comprises an amino acid sequence that is at least 80%, 85%, 90%, 95%, or 100% identical to the amino acid sequence of SEQ ID NO: 3; (6) a 4-1BB motif, which comprises an amino acid sequence that is at least 80%, 85%, 90%, 95%, or 100% identical to the amino acid sequence of SEQ ID NO: 15; (7) a CD40 motif, which comprises an amino acid sequence that is at least 80%, 85%, 90%, 95%, or 100% identical to the amino acid sequence of SEQ ID NO: 17; (8) a linker for activation of T cells (LAT) motif, which comprises an amino acid sequence that is at least 80%, 85%, 90%, 95%, or 100% identical to the amino acid sequence of SEQ ID NO: 16; and (9) a CD28 intracellular domain, which comprises an amino acid sequence that is at least 80%, 85%, 90%, 95%, or 100% identical to the amino acid sequence of SEQ ID NO: 2.

Optionally, the engineered CD3 epsilon subunit further comprises a STAT binding region within an intracellular domain thereof, which may comprise: (1) a STAT3 binding motif, which comprises an amino acid sequence that is at least 50%, 75%, or 100% identical to the amino acid sequence of SEQ ID NO: 22; or (2) a STAT5 binding motif, which comprises an amino acid sequence that is at least 50%, 75%, or 100% identical to the amino acid sequence of SEQ ID NO: 23. According to some embodiments, the STAT binding region comprises a STAT3 binding motif, comprising an amino acid sequence that is at least 50%, 75%, or 100% identical to the amino acid sequence of SEQ ID NO: 21. According to some other embodiments, the STAT binding region comprises a STAT5 binding motif, comprising an amino acid sequence that is at least 50%, 75%, or 100% identical to the amino acid sequence of SEQ ID NO: 24.

Optionally, the engineered CD3 epsilon subunit further comprises an ITAM in the intracellular domain thereof, and ITAM comprises the STAT binding region. According to some embodiments, the ITAM is a CD3z ITAM2-3 motif, which comprises an amino acid sequence that is at least 80%, 85%, 90%, 95%, or 100% identical to the amino acid sequence of SEQ ID NO: 18. According to some other embodiments, the ITAM is a CD3z ITAM3 motif, which comprises an amino acid sequence that is at least 80%, 85%, 90%, 95%, or 100% identical to the amino acid sequence of SEQ ID NO: 19.

According to some embodiments of the engineered CD3 epsilon subunit, the at least one functional region comprises both an FcεR1γ intracellular domain and an OX40 intracellular domain. Optionally, the at least one functional region comprises a first compound functional region comprising, from an N-terminus to a C-terminus direction, the FcεR1γ intracellular domain and the OX40 intracellular domain, and the first compound functional region comprises an amino acid sequence that is at least 80%, 85%, 90%, 95%, or 100% identical to the amino acid sequence of SEQ ID NO: 13. Further optionally, the at least one functional region further comprises, over the C-terminus of the first compound functional region, an ITAM (e.g., CD3z ITAM3, or CD3z ITAM2-3, etc.). Herein optionally, when the engineered CD3 zeta subunit is expressed in a population of T cells, the population of T cells exhibit an increased efficacy in vivo against tumors that comprise target cells corresponding to the population of T cells compared to when the FcεR1γ intracellular domain, the OX40 intracellular domain, and the ITAM are all absent in the engineered CD3 zeta subunit.

According to some embodiments of the engineered CD3 epsilon subunit, the at least one functional region comprises both a CD40 intracellular domain and a DAP12 intracellular domain. Optionally, the at least one functional region comprises a second compound functional region comprising, from an N-terminus to a C-terminus direction, the CD40 intracellular domain and the DAP12 intracellular domain, and the second compound functional region comprises an amino acid sequence that is at least 80%, 85%, 90%, 95%, or 100% identical to the amino acid sequence of SEQ ID NO: 14. Further optionally, the at least one functional region further comprises, at the C-terminus of the second compound functional region, an ITAM (e.g., CD3z ITAM3, or CD3z ITAM2-3, etc.).

In any of the embodiments as described above, the engineered CD3 epsilon subunit further comprises, from the N-terminus to the C-terminus thereof, a 4-1BB intracellular domain and an ITAM (e.g., CD3z ITAM3, or CD3z ITAM2-3, etc.). Herein, when the engineered CD3 epsilon subunit is expressed in a T cell that is specifically against a target cell having a specific antigen, at least one of the following is met if compared to when the 4-1BB intracellular domain and the ITAM are absent in the engineered CD3 epsilon subunit: (1) the T cell exhibits an increased cytotoxicity against the target cell in vitro; and (2) the T cell exhibits an increased stimulation by the antigen in vitro.

According to some embodiments of the engineered CD3 epsilon subunit, the at least one functional region comprises both a 4-1BB motif and an LAT motif. Further optionally, the engineered CD3 epsilon subunit further comprises an ITAM (e.g., CD3z ITAM3, or CD3z ITAM2-3, etc.) at the C-terminus thereof. Herein optionally, when the engineered CD3 epsilon subunit is expressed in a population of T cells, at least one of the following is met if compared to when the 4-1BB motif, the LAT motif and the ITAM are all absent in the engineered CD3 epsilon subunit: (1) the population of T cells comprise a higher percentage of central memory T cells; and (2) the population of T cells exhibit an increased expansion in vitro upon repeated stimulation with the antigen. Further optionally, the ITAM comprises a STAT binding region (e.g. a STAT3 binding motif, or a STAT5 binding motif, etc.).

According to some embodiments of the engineered CD3 epsilon subunit, the at least one functional region comprises both a CD40 motif and an LAT motif. Further optionally, the engineered CD3 epsilon subunit further comprises an ITAM (e.g., CD3z ITAM3, or CD3z ITAM2-3, etc.) at the C-terminus thereof. Optionally, when the engineered CD3 zeta subunit is expressed in a population of T cells, at least one of the following is met if compared to when the CD40 motif, the LAT motif, and the ITAM are all absent in the engineered CD3 zeta subunit: (1) the population of T cells exhibit an increased surface expression of the engineered CD3 epsilon subunit; (2) the population of T cells comprise a higher percentage of naïve T cells; (3) the population of T cells comprise a higher percentage of central memory T cells; (4) the population of T cells exhibit an increased efficacy in vivo against tumors that comprise the target cells; and (5) the population of T cells exhibit a higher ratio in tumor tissues infiltrated therewith in vivo.

In any of the embodiments of the engineered CD3 epsilon subunit as described above, the target-recognizing region may optionally be operably incorporated into the CD3 epsilon component.

In any of the embodiments of the engineered CD3 epsilon subunit as described above, the target-recognizing region may comprise: (1) an antigen-binding region; or (2) a ligand or a fragment thereof that binds to a cell-surface receptor expressed on a target cell of the immune cell. According to some embodiments, the target-recognizing region comprises an antigen-binding region, which may optionally comprise a single-chain variable fragment (scFv) that specifically recognizes ALPP, LYPD3, IL13Ra2, BCMA, CD16, CD19, CD20, CD22, CD27 CD138, CD33, CD123, CD171, CD70, CD7, CS-1, PSMA, PSCA, ROR1, GD2, MUC1, MUC16, HER2(ErbB2), MET, EphA2, EpCAM, CEA, CSPG4, Lewis Y antigen, Mesothelin, NKG2D, Glypican-3 (GPC-3), FAP, FRa (folate receptoralpha), EGFR, EGFR vIII, IL-11Ra (IL11 receptor alpha), VEGFR-II, B7-H6, and DNAM-1. In some specific embodiments, the antigen-binding region comprises a single-chain variable fragment (scFv) against ALPP. According to some other embodiments, the target-recognizing region comprises an antigen-binding region, which may optionally comprise a single-domain antibody (sdAb or nanobody). According to some embodiments, the target-recognizing region may comprise a ligand or a fragment thereof, and the ligand may optionally be IL13 (E13Y), IL-13, IL-11, IL-10, APRIL, GM-CSF, TPO, Adnectin, T1E, FLT3L, EPHRIN B2, CTLX, LFA-1, or FSH. In some specific embodiments, the ligand is IL13 (E13Y).

According to some embodiments of the engineered CD3 epsilon subunit, a flexible linker is between the target-recognizing region and the CD3 epsilon component, which may comprise a sequence of SEQ ID NO: 6.

In a third aspect, the present disclosure further provides an engineered TCR complex system.

According to some embodiments, the engineered TCR complex system comprises an engineered CD3 epsilon subunit according to any embodiments of the engineered CD3 epsilon subunit as described above in the second aspect. Optionally, the engineered TCR complex system may further comprise an engineered CD3 zeta subunit according to any embodiments engineered CD3 zeta subunit as described above in the first aspect.

In some embodiments of the engineered TCR complex system provided herein, the co-stimulatory region of the engineered CD3 zeta subunit comprises: (1) a co-stimulatory domain, or a functional portion or a functional variant thereof, of CD28; or (2) a co-stimulatory domain, or a functional portion or a functional variant thereof, of 4-1BB.

In embodiments of the engineered TCR complex system which comprise both an engineered CD3 epsilon subunit and engineered CD3 zeta subunit, one or both of the engineered CD3 epsilon subunit and the engineered CD3 zeta subunit may optionally comprise a STAT binding region within an intracellular domain thereof.

In embodiments of the engineered TCR complex system where both of the engineered CD3 epsilon subunit and the engineered CD3 zeta subunit comprise a STAT binding region within an intracellular domain thereof, the STAT binding region of the engineered CD3 epsilon subunit is optionally same or different from the STAT binding region of the engineered CD3 zeta subunit. In some embodiments where the STAT binding region of the engineered CD3 epsilon subunit and the STAT binding region of the engineered CD3 zeta subunit are different, the STAT binding region of the engineered CD3 epsilon subunit and the STAT binding region of the engineered CD3 zeta subunit are (1) respectively a STAT3 binding motif and a STAT5 binding motif or (2) are respectively a STAT5 binding motif and a STAT3 binding motif.

In embodiments of the engineered TCR complex system where only the engineered CD3 zeta subunit comprises a STAT binding region within an intracellular domain thereof, the STAT binding region comprises a STAT3 binding motif or a STAT5 binding motif.

In any of the embodiments of the engineered TCR complex system as described above, when the engineered CD3 epsilon subunit is expressed in a population of T cells, at least one of the following is met if compared to when the STAT binding region is absent in any of the engineered CD3 epsilon subunit or the engineered CD3 zeta subunit: (1) the population of T cells comprise a higher percentage of terminally differentiated effector T cells; (2) the population of T cells exhibit an increased antigen-specific stimulation in vitro; and (3) the population of T cells exhibit an increased expansion in vitro upon repeated stimulation with the antigen.

According to some embodiments, the engineered TCR complex system comprises an engineered CD3 zeta subunit according to any embodiments engineered CD3 zeta subunit as described above in the first aspect, and the engineered TCR complex system further comprises an engineered target-recognizing TCR subunit. Herein the engineered target-recognizing TCR subunit can be based on one of TCRα, TCRβ, TCRγ, TCRδ, CD3ε, CD3γ, or CD3δ, and preferably can be based on one of TCRα, TCRβ, CD3ε, CD3γ, or CD3δ. Herein optionally, the engineered target-recognizing TCR subunit comprises a target-recognizing region or a target-recognizing moiety, which may comprise (1) an antigen-binding region (e.g., a single-chain variable fragment (scFv) or a single-domain antibody); or (2) a ligand or a fragment thereof that binds to a cell-surface receptor expressed on a target cell of the immune cell.

In a fourth aspect, the present disclosure further provides a chimeric polypeptide, which comprises a target-recognizing region or moiety operably linked to or incorporated into one of a TCR alpha subunit, a TCR beta subunit, a CD3 gamma subunit, a CD3 delta subunit or a CD3 epsilon subunit, or a functional portion or a functional variant thereof. The target-recognizing region or moiety comprises a ligand, a functional portion, or a functional variant thereof. Herein when the chimeric polypeptide is expressed in an immune cell, the target-recognizing region of the chimeric polypeptide is capable of binding to a cell-surface receptor expressed on a target cell of the immune cell. As such, this fourth aspect substantially provides an engineered target-recognizing TCR subunit based on any one of TCRα, TCRβ, CD3ε, CD3γ, or CD3δ, where the target-recognizing region comprises a ligand moiety that allows the TCR subunit to be able, when expressed in an immune cell (e.g. T cell) to specifically recognize and bind to the cognate ligand receptor expressed on a target cell.

According to some embodiments of the chimeric polypeptide, the target-recognizing region is operably linked to or incorporated into a CD3 epsilon subunit or a functional portion or a functional variant thereof.

Herein according to some embodiments, the CD3 epsilon subunit comprises an amino acid sequence that is at least 80%, 85%, 90%, 95%, or 100% identical to the amino acid sequence of SEQ ID NO: 5; according to some other embodiments, the CD3 epsilon subunit comprises a truncated CD3 epsilon, which comprises an amino acid sequence that is at least 80%, 85%, 90%, 95%, or 100% identical to the amino acid sequence of SEQ ID NO: 10.

Optionally, the ligand is selected from IL13 (E13Y), IL-13, IL-11, IL-10, APRIL, GM-CSF, TPO, Adnectin, T1E, FLT3L, EPHRIN B2, CTLX, LFA-1, or FSH.

According to some embodiments, the ligand is IL13 (E13Y), which optionally can comprise an amino acid sequence that is at least 80%, 85%, 90%, 95%, or 100% identical to the amino acid sequence of SEQ ID NO: 4.

In a fifth aspect, the present disclosure further provides an engineered immune cell, which comprise an engineered TCR complex system according to any one of the embodiments as described above in the third aspect or a chimeric polypeptide according to any one of the embodiments as described above in the fourth aspect. Herein optionally, the engineered immune cell can be a T lymphocyte (or T cell), a tumor infiltrating lymphocyte (TIL), or a natural killer (NK) cell.

In a sixth aspect, the present disclosure further provides a method for treating a subject in need thereof, comprising administrating to the subject a therapeutically effective amount of the engineered immune cell according to any one of the embodiments as described above in the fifth aspect. Herein the subject can be a human, but can also be a mammal such as a dog, a cat, a monkey.

As used herein and throughout the disclosure elsewhere, “a” or “an” means “at least one” or “one or more.” In one example, in the engineered CD3 zeta (CD3z) subunit as provided above in the first aspect, which comprises “a co-stimulatory region” that is operably linked to or incorporated into a CD3 zeta component, the phrase “a co-stimulatory region” can be interpreted to mean “at least one co-stimulatory region”.

In one aspect, the disclosure is related to an engineered CD3 zeta (CD3z) subunit, comprising a co-stimulatory region operably linked to or incorporated into a CD3 zeta component, in some embodiments, the CD3 zeta component comprises a human CD3 zeta or a functional portion or a functional variant thereof, comprising a sequence that has at least 80% sequence identity to SEQ ID NO: 1; and the co-stimulatory region is within an intracellular domain of the engineered CD3 zeta subunit. In some embodiments, when expressed in an immune cell, at least one of the following is met: (1) the immune cell expressing the engineered CD3 zeta subunit has a reduced activation in the absence of antigen stimulation compared to when the immune cell does not express the engineered CD3 zeta subunit; (2) the immune cell expressing the engineered CD3 zeta subunit has a reduced cytotoxicity against non-target cells compared to when the immune cell does not express the engineered CD3 zeta subunit; (3) the immune cell expressing the engineered CD3 zeta subunit has an increased activation upon antigen stimulation compared to when the immune cell does not express the engineered CD3 zeta subunit; and (4) the immune cell expressing the engineered CD3 zeta subunit has an increased immune cell response against target cells corresponding thereto compared to when the immune cell does not express the engineered CD3 zeta subunit. In some embodiments, the CD3 zeta component comprises an amino acid sequence as set forth in SEQ ID NO: 1. In some embodiments, the co-stimulatory region comprises a co-stimulatory domain, or a functional portion or a functional variant thereof, of a protein selected from the group consisting of CD28, 4-1BB, OX40, CD2, CD27, CDS, ICAM-1, LFA-1, and ICOS. In some embodiments, the co-stimulatory region comprises a co-stimulatory domain, or a functional portion or a functional variant thereof, of CD28, comprising a sequence that has at least 80% sequence identity to SEQ ID NO: 2. In some embodiments, the co-stimulatory region comprises a co-stimulatory domain, or a functional portion or a functional variant thereof, of 4-1BB, comprising a sequence that has at least 80% sequence identity to SEQ ID NO: 3. In some embodiments, the co-stimulatory region is operably fused to the C-terminus of the CD3 zeta component. In some embodiments, the co-stimulatory region is located between a transmembrane domain and an intracellular domain of the CD3 zeta component.

In some embodiments, the engineered CD3 zeta subunit described herein further comprises a STAT binding region within an intracellular domain thereof. In some embodiments, the STAT binding region comprises a STAT3 binding motif that comprises an amino acid sequence that is at least 50%, 75%, or 100% identical to the amino acid sequence of SEQ ID NO: 22. In some embodiments, the STAT3 binding motif comprises an amino acid sequence that is at least 50%, 75%, or 100% identical to the amino acid sequence of SEQ ID NO: 21. In some embodiments, the STAT binding region comprises a STAT5 binding motif that comprises an amino acid sequence that is at least 50%, 75%, or 100% identical to the amino acid sequence of SEQ ID NO: 23. In some embodiments, the STAT5 binding motif comprises an amino acid sequence that is at least 50%, 75%, or 100% identical to the amino acid sequence of SEQ ID NO: 24. In some embodiments, the STAT binding region is located within a region of the CD3 zeta component corresponding to positions 150-164 of SEQ ID NO: 1. In some embodiments, the STAT binding region is an insertion at a location of the CD3 zeta component corresponding to between position 157 and position 158 of SEQ ID NO: 1. In some embodiments, the CD3 zeta component comprises an amino acid sequence that is at least 80%, 85%, 90%, 95%, or 100% identical to the amino acid sequence of SEQ ID NO: 20. In some embodiments, the CD3 zeta component comprises an amino acid sequence that is at least 80%, 85%, 90%, 95%, or 100% identical to the amino acid sequence of SEQ ID NO: 29.

In some embodiments, when the engineered CD3 zeta subunit is expressed in a population of T cells, at least one of the following is met if compared to when the STAT binding region is absent in the engineered CD3 zeta subunit: the population of T cells exhibit an increased expansion in vitro upon repeated stimulation with the antigen; the population of T cells exhibit an increased efficacy in vivo against tumors that comprise the target cells; and the population of T cells exhibit a higher ratio in tumor tissues infiltrated therewith in vivo.

In some embodiments, the immune cell is a T lymphocyte, a tumor-infiltrating lymphocyte (TIL), or a natural kill (NK) cell. In some embodiments, when the engineered CD3 zeta subunit is expressed in a population of T cells expressing a T cell receptor (TCR), the population of T cells exhibit an increased surface expression compared to when the co-stimulatory region is absent in the engineered CD3 zeta subunit.

In one aspect, the disclosure is related to a method for modulating activities of an immune cell, comprising: expressing in the immune cell the engineered CD3 zeta subunit described herein. In some embodiments, the immune cell expressing the engineered CD3 zeta subunit: (1) has a reduced activation in the absence of antigen stimulation compared to when the immune cell does not express the engineered CD3 zeta subunit; (2) has a reduced cytotoxicity against non-target cells compared to when the immune cell does not express the engineered CD3 zeta subunit; (3) has an increased activation upon antigen stimulation compared to when the immune cell does not express the engineered CD3 zeta subunit; or (4) has an increased immune cell response against target cells corresponding thereto compared to when the immune cell does not express the engineered CD3 zeta subunit.

In one aspect, the disclosure is related to an engineered CD3 epsilon (CD3e) subunit, comprising a target-recognizing region and a CD3 epsilon component, in some embodiments, the target-recognizing region is operably fused to an extracellular domain of the engineered CD3 epsilon subunit; and the CD3 epsilon component comprises a truncated CD3 epsilon with a truncation at a region of a human CD3 epsilon subunit corresponding to the intercellular domain thereof. In some embodiments, the truncation is within a region of the human CD3 epsilon subunit corresponding to positions 151-179 of SEQ ID NO: 5. In some embodiments, the CD3 epsilon component comprises an amino acid sequence that is at least 80%, 85%, 90%, 95%, or 100% identical to the amino acid sequence of SEQ ID NO: 10. In some embodiments, the CD3 epsilon component comprises the amino acid sequence of SEQ ID NO: 10. In some embodiments, when the engineered CD3 epsilon subunit is expressed in a population of T cells, the population of T cells exhibit an increased surface expression of the engineered CD3 epsilon subunit and/or an increased expansion when compared to when the CD3 epsilon component comprises a full-length CD3 epsilon subunit. In some embodiments, the C-terminus of the CD3 epsilon component is the C-terminus of the engineered CD3 epsilon subunit. In some embodiments, when the engineered CD3 epsilon subunit is expressed in a population of T cells, at least one of the following is met: (1) the population of T cells exhibit an increased surface expression of the engineered CD3 epsilon subunit, comparing to when the CD3 epsilon component comprises a full-length CD3 epsilon subunit; (2) the population of T cells comprise a higher percentage of central memory T cells, comparing to when the CD3 epsilon component comprises a full-length CD3 epsilon subunit; and (3) the population of T cells exhibit an increased expansion in vitro upon repeated stimulation with the antigen, comparing to when the CD3 epsilon component comprises a full-length CD3 epsilon subunit.

In some embodiments, the engineered CD3 epsilon subunit described herein further comprises at least one functional region in an intracellular domain thereof, in some embodiments, the at least one functional region is operably fused to the C-terminus of the CD3 epsilon component.

In some embodiments, the at least one functional region comprises an immune receptor tyrosine based activation motif (ITAM). In some embodiments, the ITAM is derived from CD3z, CD3g, DAP12, FcαRI, FcγRI, FcγRII, FcγRIII, Dectin-1, CLEC-1, CD28, or CD72. In some embodiments, the ITAM comprises: (1) a CD3z ITAM3 that comprises an amino acid sequence that is at least 80%, 85%, 90%, 95%, or 100% identical to the amino acid sequence of SEQ ID NO: 11; or (2) a CD3z ITAM2-3 that comprises an amino acid sequence that is at least 80%, 85%, 90%, 95%, or 100% identical to the amino acid sequence of SEQ ID NO: 12. In some embodiments, the ITAM is located at the C-terminus of the engineered CD3 epsilon subunit. In some embodiments, the ITAM is operably fused to the C-terminus of the CD3 epsilon component, in some embodiments, the ITAM comprises a CD3z ITAM3 or a CD3z ITAM2-3. In some embodiments, the ITAM comprises a STAT binding region, in some embodiments, the STAT binding region is a STAT3 binding motif or a STAT5 binding motif.

In some embodiments, when the engineered CD3 epsilon subunit is expressed in a population of T cells, the population of T cells comprise a higher percentage of central memory T cells compared to when the ITAM is absent in the engineered CD3 epsilon subunit.

In some embodiments, the at least one functional region comprises any one or a combination of: (1) an FcεR1γ intracellular domain, which comprises an amino acid sequence that is at least 80%, 85%, 90%, 95%, or 100% identical to the amino acid sequence of SEQ ID NO: 25; (2) an OX40 intracellular domain, which comprises an amino acid sequence that is at least 80%, 85%, 90%, 95%, or 100% identical to the amino acid sequence of SEQ ID NO: 26; (3) a CD40 intracellular domain, which comprises an amino acid sequence that is at least 80%, 85%, 90%, 95%, or 100% identical to the amino acid sequence of SEQ ID NO: 27; (4) a DAP12 intracellular domain, which comprises an amino acid sequence that is at least 80%, 85%, 90%, 95%, or 100% identical to the amino acid sequence of SEQ ID NO: 28; (5) a 4-1BB intracellular domain, which comprises an amino acid sequence that is at least 80%, 85%, 90%, 95%, or 100% identical to the amino acid sequence of SEQ ID NO: 3; (6) a 4-1BB motif, which comprises an amino acid sequence that is at least 80%, 85%, 90%, 95%, or 100% identical to the amino acid sequence of SEQ ID NO: 15; (7) a CD40 motif, which comprises an amino acid sequence that is at least 80%, 85%, 90%, 95%, or 100% identical to the amino acid sequence of SEQ ID NO: 17; (8) a linker for activation of T cells (LAT) motif, which comprises an amino acid sequence that is at least 80%, 85%, 90%, 95%, or 100% identical to the amino acid sequence of SEQ ID NO: 16; and (9) a CD28 intracellular domain, which comprises an amino acid sequence that is at least 80%, 85%, 90%, 95%, or 100% identical to the amino acid sequence of SEQ ID NO: 2.

In some embodiments, the engineered CD3 epsilon subunit described herein further comprises a STAT binding region within an intracellular domain thereof. In some embodiments, the STAT binding region comprises: (1) a STAT3 binding motif, which comprises an amino acid sequence that is at least 50%, 75%, or 100% identical to the amino acid sequence of SEQ ID NO: 22; or (2) a STAT5 binding motif, which comprises an amino acid sequence that is at least 50%, 75%, or 100% identical to the amino acid sequence of SEQ ID NO: 23. In some embodiments, the STAT binding region comprises a STAT3 binding motif, which comprises an amino acid sequence that is at least 50%, 75%, or 100% identical to the amino acid sequence of SEQ ID NO: 21. In some embodiments, the STAT binding region comprises a STAT5 binding motif, which comprises an amino acid sequence that is at least 50%, 75%, or 100% identical to the amino acid sequence of SEQ ID NO: 24. In some embodiments, the engineered CD3 epsilon subunit described herein further comprises an ITAM in the intracellular domain thereof, in some embodiments, ITAM comprises the STAT binding region. In some embodiments, the ITAM is a CD3z ITAM2-3 motif, which comprises an amino acid sequence that is at least 80%, 85%, 90%, 95%, or 100% identical to the amino acid sequence of SEQ ID NO: 18. In some embodiments, the ITAM is a CD3z ITAM3 motif, which comprises an amino acid sequence that is at least 80%, 85%, 90%, 95%, or 100% identical to the amino acid sequence of SEQ ID NO: 19. In some embodiments, the at least one functional region comprises both an FcεR1γ intracellular domain and an OX40 intracellular domain. In some embodiments, the at least one functional region comprises a first compound functional region comprising, from an N-terminus to a C-terminus direction, the FcεR1γ intracellular domain and the OX40 intracellular domain, in some embodiments, the first compound functional region comprises an amino acid sequence that is at least 80%, 85%, 90%, 95%, or 100% identical to the amino acid sequence of SEQ ID NO: 13. In some embodiments, the at least one functional region further comprises, over the C-terminus of the first compound functional region, an ITAM, in some embodiments, the ITAM comprises CD32 ITAM3 or CD3z ITAM2-3.

In some embodiments, when the engineered CD3 zeta subunit is expressed in a population of T cells, the population of T cells exhibit an increased efficacy in vivo against tumors that comprise the target cells compared to when the FcεR1γ intracellular domain, the OX40 intracellular domain, and the ITAM are all absent in the engineered CD3 zeta subunit.

In some embodiments, the at least one functional region comprises both a CD40 intracellular domain and a DAP12 intracellular domain. In some embodiments, the at least one functional region comprises a second compound functional region comprising, from an N-terminus to a C-terminus direction, the CD40 intracellular domain and the DAP12 intracellular domain, in some embodiments, the second compound functional region comprises an amino acid sequence that is at least 80%, 85%, 90%, 95%, or 100% identical to the amino acid sequence of SEQ ID NO: 14. In some embodiments, the at least one functional region further comprises, at the C-terminus of the second compound functional region, an ITAM, in some embodiments, the ITAM comprises a CD3z ITAM3 or a CD3z ITAM2-3.

In some embodiments, the engineered CD3 epsilon subunit described herein further comprises, from the N-terminus to the C-terminus thereof, a 4-1BB intracellular domain and an ITAM, in some embodiments, the ITAM comprises a CD3z ITAM3 or a CD3z ITAM2-3. In some embodiments, when the engineered CD3 epsilon subunit is expressed in a T cell that is specifically against a target cell having a specific antigen, at least one of the following is met if compared to when the 4-1BB intracellular domain and the ITAM are absent in the engineered CD3 epsilon subunit: the T cell exhibits an increased cytotoxicity against the target cell in vitro; and the T cell exhibits an increased stimulation by the antigen in vitro.

In some embodiments, the at least one functional region comprises both a 4-1BB motif and an LAT motif. In some embodiments, the engineered CD3 epsilon subunit described herein further comprises an ITAM at the C-terminus of the engineered CD3 epsilon subunit, in some embodiments, the ITAM comprises a CD3z ITAM3 or a CD3z ITAM2-3. In some embodiments, when the engineered CD3 epsilon subunit is expressed in a population of T cells, at least one of the following is met if compared to when the 4-1BB motif, the LAT motif and the ITAM are all absent in the engineered CD3 epsilon subunit: the population of T cells comprise a higher percentage of central memory T cells; and the population of T cells exhibit an increased expansion in vitro upon repeated stimulation with the antigen. In some embodiments, the ITAM comprises a STAT binding region, in some embodiments, the STAT binding region is a STAT3 binding motif or a STAT5 binding motif.

In some embodiments, the at least one functional region comprises both a CD40 motif and an LAT motif. In some embodiments, the engineered CD3 epsilon subunit described herein further comprises an ITAM at the C-terminus of the engineered CD3 epsilon subunit, in some embodiments, the ITAM comprises a CD3z ITAM3 or a CD3z ITAM2-3. In some embodiments, when the engineered CD3 zeta subunit is expressed in a population of T cells, at least one of the following is met if compared to when the CD40 motif, the LAT motif, and the ITAM are all absent in the engineered CD3 zeta subunit: the population of T cells exhibit an increased surface expression of the engineered CD3 epsilon subunit; the population of T cells comprise a higher percentage of naïve T cells; the population of T cells comprise a higher percentage of central memory T cells; the population of T cells exhibit an increased efficacy in vivo against tumors that comprise the target cells; and the population of T cells exhibit a higher ratio in tumor tissues infiltrated therewith in vivo.

In some embodiments, the target-recognizing region is operably incorporated into the CD3 epsilon component. In some embodiments, the target-recognizing region comprises: (1) an antigen-binding region; or (2) a ligand or a fragment thereof that binds to a cell-surface receptor expressed on a target cell of the immune cell. In some embodiments, the antigen-binding region comprises a single-chain variable fragment (scFv) that specifically recognizes ALPP, LYPD3, IL13Ra2, BCMA, CD16, CD19, CD20, CD22, CD27 CD138, CD33, CD123, CD171, CD70, CD7, CS-1, PSMA, PSCA, ROR1, GD2, MUC1, MUC16, HER2(ErbB2), MET, EphA2, EpCAM, CEA, CSPG4, Lewis Y antigen, Mesothelin, NKG2D, Glypican-3 (GPC-3), FAP, FRa (folate receptoralpha), EGFR, EGFR vIII, IL-11Ra (IL11 receptor alpha), VEGFR-II, B7-H6, and DNAM-1. In some embodiments, the antigen-binding region comprises a single-chain variable fragment (scFv) against ALPP. In some embodiments, the antigen-binding region comprises a single-domain antibody (sdAb or nanobody). In some embodiments, the ligand is selected from the group consisting of IL13 (E13Y), IL-13, IL-11, IL-10, APRIL, GM-CSF, TPO, Adnectin, T1E, FLT3L, EPHRIN B2, CTLX, LFA-1, and FSH. In some embodiments, the ligand is IL13 (E13Y).

In some embodiments, the engineered CD3 epsilon subunit described herein further comprises a flexible linker between the target-recognizing region and the CD3 epsilon component. In some embodiments, the flexible linker comprises a sequence of SEQ ID NO: 6.

In one aspect, the disclosure is related to an engineered TCR complex system, comprising at least one of: the engineered CD3 zeta subunit described herein; and the engineered CD3 epsilon subunit described herein. In some embodiments, the engineered TCR complex system described herein comprises both the engineered CD3 epsilon subunit and the engineered CD3 zeta subunit described herein.

In some embodiments, the co-stimulatory region of the engineered CD3 zeta subunit comprises: a co-stimulatory domain, or a functional portion or a functional variant thereof, of CD28; or a co-stimulatory domain, or a functional portion or a functional variant thereof, of 4-1BB. In some embodiments, one or both of the engineered CD3 epsilon subunit and the engineered CD3 zeta subunit comprise a STAT binding region within an intracellular domain thereof. In some embodiments, both of the engineered CD3 epsilon subunit and the engineered CD3 zeta subunit comprise a STAT binding region within an intracellular domain thereof, in some embodiments, the STAT binding region of the engineered CD3 epsilon subunit is different from the STAT binding region of the engineered CD3 zeta subunit. In some embodiments, the STAT binding region of the engineered CD3 epsilon subunit and the STAT binding region of the engineered CD3 zeta subunit are (1) respectively a STAT3 binding motif and a STAT5 binding motif or (2) are respectively a STAT5 binding motif and a STAT3 binding motif. In some embodiments, only the engineered CD3 zeta subunit comprises a STAT binding region within an intracellular domain thereof, in some embodiments, the STAT binding region comprises a STAT3 binding motif or a STAT5 binding motif. In some embodiments, when the engineered CD3 epsilon subunit is expressed in a population of T cells, at least one of the following is met if compared to when the STAT binding region is absent in any of the engineered CD3 epsilon subunit or the engineered CD3 zeta subunit: the population of T cells comprise a higher percentage of terminally differentiated effector T cells; the population of T cells exhibit an increased antigen-specific stimulation in vitro; and the population of T cells exhibit an increased expansion in vitro upon repeated stimulation with the antigen. In some embodiments, the engineered TCR complex system described herein comprises the engineered CD3 zeta subunit, and further comprising an engineered target-recognizing TCR subunit, in some embodiments, the TCR subunit is based on one of TCRα, TCRβ, TCRγ, TCRδ, CD3ε, CD3γ, or CD3δ, and preferably on one of TCRα, TCRβ, CD3ε, CD3γ, or CD3δ. In some embodiments, the engineered target-recognizing TCR subunit comprises a target-recognizing region, the target-recognizing region comprising: (1) an antigen-binding region, the antigen-binding region comprising at least one of a single-chain variable fragment (scFv) or a single-domain antibody; or (2) a ligand or a fragment thereof that binds to a cell-surface receptor expressed on a target cell of the immune cell.

In one aspect, the disclosure is related to a chimeric polypeptide, comprising a target-recognizing region operably linked to or incorporated into one of a TCR alpha subunit, a TCR beta subunit, a CD3 gamma subunit, a CD3 delta subunit or a CD3 epsilon subunit, or a functional portion or a functional variant thereof, in some embodiments, the target-recognizing region comprises a ligand, a functional portion, or a functional variant thereof, in some embodiments, when the chimeric polypeptide is expressed in an immune cell, the target-recognizing region of the chimeric polypeptide is capable of binding to a cell-surface receptor expressed on a target cell of the immune cell. In some embodiments, the target-recognizing region is operably linked to or incorporated into a CD3 epsilon subunit or a functional portion or a functional variant thereof. In some embodiments, the CD3 epsilon subunit comprises an amino acid sequence that is at least 80%, 85%, 90%, 95%, or 100% identical to the amino acid sequence of SEQ ID NO: 5. In some embodiments, the CD3 epsilon subunit comprises a truncated CD3 epsilon, which comprises an amino acid sequence that is at least 80%, 85%, 90%, 95%, or 100% identical to the amino acid sequence of SEQ ID NO: 10. In some embodiments, the ligand is selected from the group consisting of IL13 (E13Y), IL-13, IL-11, IL-10, APRIL, GM-CSF, TPO, Adnectin, T1E, FLT3L, EPHRIN B2, CTLX, LFA-1, and FSH. In some embodiments, the ligand is IL13 (E13Y). In some embodiments, the IL13 (E13Y) ligand comprises an amino acid sequence that is at least 80%, 85%, 90%, 95%, or 100% identical to the amino acid sequence of SEQ ID NO: 4.

In one aspect, the disclosure is related to an engineered immune cell, comprising: the engineered TCR complex system described herein; and/or a chimeric polypeptide described herein. In some embodiments, the engineered immune cell is a T lymphocyte, a tumor infiltrating lymphocyte (TIL), or a natural killer (NK) cell.

In one aspect, the disclosure is related to a method for treating a subject in need thereof, comprising administrating to the subject a therapeutically effective amount of the engineered immune cell described herein.

As used herein, the term “immune cells” can mean any of T lymphocytes (including αβ T cells or γδ T cells), tumor-infiltrating lymphocytes (TILs), natural killer (NK) cells, or NK T cells, or any of these above cells that have been engineered (e.g. cells expressing TCR or Chimeric antigen receptor (CAR)). In the Examples provided in the disclosure, the T lymphocytes, or T cells, are used as illustrating yet non-limiting example for the immune cells.

As used herein, “single-chain variable fragment” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

As used herein, the term “TCR signaling complex” or “TCR complex” refers to a complex formed between TCR subunits (preferably TCRα and TCRβ, but can also include TCRδ and TCRγ), and CD3 complex subunits (CD3ε, CD3γ, CD3δ, and CD3ζ); the term “TCR signaling complex subunit” or “TCR subunit” used herein refers to a subunit of the TCR complex, which include, for example, TCRα, TCRβ, TCRγ, TCRδ, CD3ε, CD3γ, CD3δ, and CD3ζ. One or more of the TCR subunits may be an engineered TCR subunit provided by the disclosure.

As used herein, the term “STAT binding region” includes, but is not limit to, a STAT3 binding motif (e.g., SEQ ID NO: 22) and/or a STAT5 binding motif (e.g., SEQ ID NO: 23). In some embodiments, the STAT binding region includes one STAT3 or STAT5 binding motif. In some embodiments, the STAT binding region includes two or more STAT3 or STAT5 binding motifs.

As used here, the term “immunoreceptor tyrosine-based activation motif (ITAM)” refers to a conserved sequence of four amino acids that is repeated twice and is present in the cytoplasmic tails (i.e. endodomains) of certain cell surface proteins of the immune system. Details of ITAM can be found, e.g., in Love, P. E., et al. “ITAM-mediated signaling by the T-cell antigen receptor.” Cold Spring Harbor Perspectives in Biology 2.6 (2010): a002485, which is incorporated by reference in its entirety.

As used herein, the term “expansion capability” refers to the capability of immune cells (e.g., immune cells expressing any of the engineered TCR complexes described herein) to proliferate (e.g., after about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days post transfection). In some embodiments, “increased expansion capability” indicates no less than 1.1-fold (i.e. 110% or more) increase of the proliferation rate of reference immune cells that are not transduced to express the corresponding subunits.

As used herein, the term “cytolytic toxicity” refers to the capability to of immune cells to kill specific target cells when the immune cells are co-cultured with the specific target cells. As used herein, the term “specific target cells” refer to cells that express antigens that can be specifically recognized by the CAR or TCR (e.g., Aspire-TCR) on the immune cells. In some embodiments, “increased cytolytic toxicity” indicates no less than 1.1-fold (i.e. 110% or more) increase of killing efficiency of specific target cells as compared to using control immune cells (e.g., non-transduced immune cells).

As used herein, the term “Aspire-TCR” can be any Aspire-TCRs constructs described herein.

As used herein, the term “increase”, “increased”, “reduce”, or “reduced”, “alter”, “altered”, “change”, “changed”, “higher”, “lower”, or alike, refers to the level change of no less than 10% compared to a reference level.

As used herein, the phrase “substantially unchanged” means that a level of a variable under examination (such as the expression level of the target CAR/TCR) changes by less than 5% if comparing a later timepoint with an earlier reference timepoint. If a change is greater than or equal to 5%, such a change can be deemed as an “increase” or “reduce” as mentioned herein.

As used herein, the phrase “background activation” or “basal activation” refers to the activation level of immune cells when no corresponding target cells are present. In some embodiments, the target cells can express one or more types of target molecules that can be specifically recognized by a target-recognizing region (e.g., any of the target-recognizing regions described herein) expressed by the immune cells.

As used herein, the phrase “target cells” refers to cells that can be specifically targeted or killed by immune cells.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows the structural diagram of a conventional TCR signaling complex.

FIGS. 2A-2C respectively show the structural diagram of an engineered CD3 zeta subunit (FIG. 2A), an engineered CD3 epsilon subunit (FIG. 2B), and a ligand-based target-recognizing Aspire-TCR subunit (FIG. 2C) according to some embodiments of the disclosure.

FIG. 3 shows structural diagrams of four Aspire-TCR constructs involved in the experiments.

FIGS. 4A-4E show the IL13 expression on different Aspire-T cells, including NT cells (FIG. 4A), CD3e (FIG. 4B), BBe (FIG. 4C), CD3e-28z (FIG. 4D), and CD3e-BBz (FIG. 4E).

FIG. 5 shows the specific cytotoxicity of IL13(E13Y) Aspire-T cells against target tumor cells (U251 MG glioma cell line).

FIG. 6 shows IFN-γ production of the activated IL13(E13Y) Aspire-T cells against tumor cells.

FIG. 7A shows the expression level of IL13Ra1 in THP1 cells.

FIG. 7B shows the expression level of IL13Ra2 in THP1 cells.

FIG. 7C shows the cytotoxicity of IL13(E13Y) Aspire-T cells against non-target THP-1 cells.

FIG. 8A shows the percentage of the IFN-γ-producing cells in the presence (“THP-1”) or absence (“No target”) of THP1 cells. Activation of IL13(E13Y) Aspire-T cells against the non-target THP-1 cells was assessed.

FIG. 8B shows the IFN-γ secretion levels of the activated IL13(E13Y) Aspire-T cells in the presence of the non-target THP-1 cells.

FIG. 9 shows the structural diagrams of two anti-ALPP Aspire-TCR constructs.

FIGS. 10A-10C show the expression of F8 scFv on different cells, including non-transduced T cells (“NT”; FIG. 10A), F8-BBe Aspire-T cells (FIG. 10B), and F8-28z Aspire-T cells (FIG. 10C).

FIGS. 11A-11B show the specific cytotoxicity of F8 Aspire-T cells against target tumor cells, including SiHa cells (FIG. 11A) and Caski cells (FIG. 11B).

FIG. 12 shows IFN-γ secretion levels of the activated F8 Aspire-T cells in the presence of target tumor cells.

FIG. 13A shows the expression level of ALPP ligand in A549 cells.

FIG. 13B shows the expression level of ALPP ligand in SiHa cells.

FIG. 13C shows the background cytotoxicity of F8 Aspire-T cells against the non-target A549 cells.

FIG. 14 shows the various DNA constructs encoding the Aspire-TCR designs A-K.

FIGS. 15A-15K show the Aspire-TCR complex structures of the designs A-K once expressed in a T cell, respectively.

FIGS. 16A-16D show the scFv expression among different Aspire-T cells including Aspire-T cells expressing scFv-CD3e-28z (FIG. 16B), scFv-ΔCD3e-28z (FIG. 16C), or scFv-ΔCD3eZ-28z (FIG. 16D) in comparison to the control NT T cells (FIG. 16A).

FIGS. 17A-17F show the scFv expression among different Aspire-T cells including Aspire-T cells expressing scFv-ΔCD3e-FOZ-28z (FIG. 17D), scFv-ΔCD3e-40DZ-28z (FIG. 17E), or scFv-ΔCD3e-41BBZ-28z (FIG. 17F) in comparison to the control NT T cells (FIG. 17A) and Aspire-T cells expressing scFv-CD3e-28z (FIG. 17B) or scFv-ΔCD3e-28z (FIG. 17C).

FIGS. 18A-18F show the scFv expression among different Aspire-T cells including Aspire-T cells expressing scFv-ΔCD3e-41BBM-M1Z-28z (FIG. 18B), scFv-ΔCD3e-41BBM-M1Z-28z (FIG. 18C), scFv-ΔCD3e-28z-S3 (FIG. 18D), scFv-ΔCD3e-S5-28z-S3 (FIG. 18E), or scFv-ΔCD3e-41BBM-M1Z-S5-28z-S3 (FIG. 18F) in comparison to Aspire-T cells expressing scFv-ΔCD3e-28z (FIG. 18A).

FIGS. 19A-19C show the scFv Aspire-T cells expansion in vitro.

FIGS. 20A-20B show the proportion of memory T cells in different scFv Aspire-T cells after expansion.

FIGS. 21A-21B show the specific cytotoxicity of scFv Aspire-T cells against target SiHa tumor cells.

FIGS. 22A-22B show the activation of scFv Aspire-T cells upon antigen-specific stimulation of target SiHa tumor cells.

FIGS. 23A-23B show the expansion of CAR+ T cells derived from various scFv Aspire-T cells in response to the simulation of tumor cells.

FIG. 24 shows the structural diagrams of IE06, where “CD8a” stands for the signal peptide from CD8A corresponding to amino acids 1-46 of SEQ ID NO: 31, “hIL12p40” stands for human IL12 beta subunit corresponding to amino acids 47-352 of SEQ ID NO: 31, “hIL12p35” stands for human IL12 alpha subunit corresponding to amino acids 368-564 of SEQ ID NO: 31, and “TM” stands for transmembrane domain corresponding to amino acids 565-610 of SEQ ID NO: 31. A linker is between the “hIL12p40” and the “hIL12p35”, corresponding to amino acids 353-367 of SEQ ID NO: 31.

FIG. 25 shows the in vivo tumor volume in NSG mice that were transplanted with tumor cells and treated with armored Aspire-T cells.

FIG. 26 shows the in vivo IE06-armored scFv Aspire-T cell proliferation using a tumor cell transplantation model in NSG mice. scFv Aspire-T cell proliferation is represented by hCD45: mCD45 ratio in peripheral blood of NSG mice. scFv Aspire-T cells were generated by transducing human peripheral T cells with the various DNA constructs encoding various Aspire-TCR designs.

FIG. 27 shows armored scFv Aspire-T cell infiltration in tumors from NSG mice that were transplanted with tumor cells. scFv Aspire-T cell infiltration is represented by hCD45: mCD45 ratio in tumor tissues.

FIG. 28 lists the relevant amino acid sequences discussed in the disclosure.

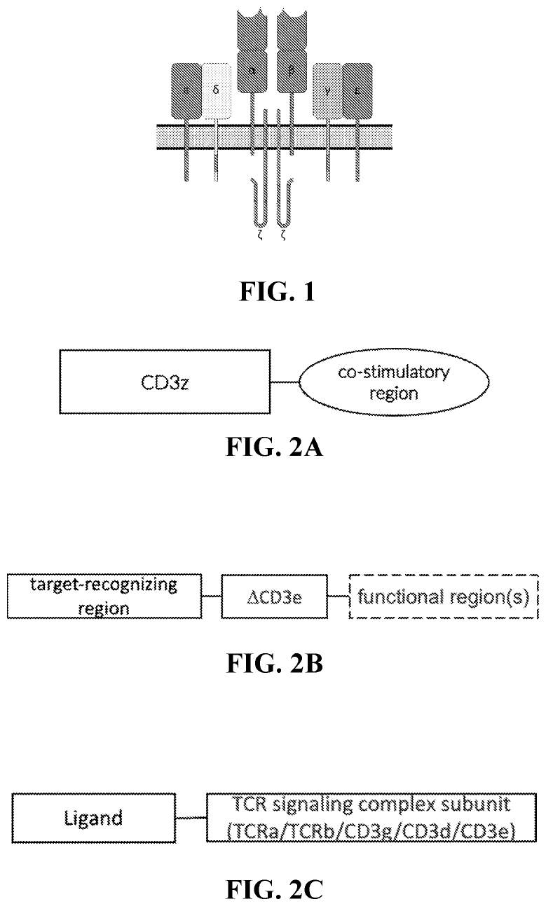

### DETAILED DESCRIPTION

The present disclosure provides an engineered T cell receptor (TCR) signaling complex termed “Antigen-specificity redirected TCR” complex (or “Aspire-TCR”), which comprises one or more engineered TCR signaling subunits that are based on TCRα, TCRβ, TCRγ, TCRδ, CD3ε, CD3γ, CD3δ, or CD3ζ. In particular, these engineered TCR signaling subunits may include an engineered CD3 zeta (i.e. CD3ζ or CD3z) subunit, and/or an engineered target-recognizing subunit based on any one of TCRα, TCRβ, TCRγ, TCRδ, CD3ε, CD3γ, and CD3ζ.

On the one hand, an engineered CD3 zeta subunit is provided, which substantially comprises a co-stimulatory region (e.g., the co-stimulatory domain of CD28, 4-1BB, OX40, etc.) operably linked to or incorporated into the intracellular domain of a CD3 zeta component (e.g., a human CD3z subunit). Expression of the engineered CD3 zeta subunit in the immune cells (e.g., T cells) can result in improved characteristics, such as an enhanced surface expression of a TCR signaling complex (esp. surface expression of a specific TCR heterodimer, or of an engineered target-recognizing TCRα/TCRβ/TCRγ/TCRδ/CD3ε/CD3γ/CD3δ co-expressed therewith), a reduced basal/background activation in the absence of antigen stimulation, a reduced cytotoxicity against non-target cells, an increased activation upon antigen stimulation, and/or an increased immune cell response against target cells, etc., of the immune cells.

The engineered CD3z subunit may be modified to further comprise a STAT (e.g., STAT3 or STAT5) binding region, allowing the immune cells expressing such engineered CD3z subunit to have an increased expansion capability upon repeated antigen stimulation, an increased in vivo antitumor efficacy, and/or an increased tumor tissue infiltration capability.

On the other hand, an engineered target-recognizing subunit is further provided, which substantially comprises a target-recognizing moiety (i.e. target-recognizing region) operably linked to or operably incorporated into the extracellular domain of a TCR signaling complex subunit (i.e. TCR subunit), which can be any one of TCRα, TCRβ, CD3γ, CD3δ, and CD3ε. Depending on different embodiments provided by the disclosure, the target-recognizing moiety may comprise a single-chain variable fragment (scFv) or a single-domain antibody (sdAb or nanobody) that can specifically recognize and bind to an epitope of a corresponding antigen expressed or presented on surfaces of specific target cells, or may comprise a ligand moiety (i.e. ligand region) that can specifically recognize and bind to a cognate receptor (i.e. the corresponding ligand receptor) expressed on the surface of specific target cells. Expression of such engineered target-recognizing subunit in immune cells (e.g., T cells) confers such engineered immune cells with specific and efficient cytotoxicity against the target cells that expressing these target molecules (i.e. antigens or cognate receptors).

In particular, the present disclosure provides an engineered target-recognizing CD3 epsilon subunit, in which the target-recognizing moiety (e.g., scFv, sdAb, or ligand) is operably fused to the N-terminus of a CD3 epsilon component that has a truncation at a region corresponding to an intracellular domain thereof (i.e. ΔCD3e), and further optionally, the C-terminus of the CD3 epsilon component (i.e. ΔCD3e) is operably connected with one or more functional regions (e.g., immune receptor tyrosine based activation motif (ITAM), CD28 intracellular domain, CD40 intracellular domain, 4-1 BB motif, STAT binding region, etc.). Depending on different embodiments of the engineered CD3 epsilon subunits (i.e. with different functional region(s)), they may confer the immune cells expressing the same with altered or improved characteristics such as an increased surface expression of the TCR signaling complex, an increased expansion capability upon repeated antigen stimulation, an altered percentage of memory T cells, an increased in vivo antitumor efficacy, and/or an increased tumor tissue infiltration capability, etc.

More details for the above-mentioned Aspire-TCR subunits are provided below.

### Engineered CD3 Zeta Subunit

In one aspect, the disclosure provides an engineered CD3 zeta (CD3z) subunit, which substantially comprises a co-stimulatory region operably linked to or incorporated into a CD3 zeta component. Herein, the CD3 zeta component comprises a mammalian CD3 zeta or a functional portion or a functional variant thereof, and the co-stimulatory region is within an intracellular domain of the engineered CD3 zeta subunit.

As used herein, the term “engineered CD3 zeta”, “engineered CD3 zeta subunit” or “engineered CD3 zeta subunit” is referred to as a mammalian CD3 zeta subunit-based polypeptide, or a functional portion or a functional variant thereof that substantially maintains the CD3 zeta functionality, e.g. capable of being incorporated into the TCR complex and/or mediating the TCR intracellular signaling. One example of the mammalian CD3 zeta subunit is a human CD3 zeta subunit, which will be used as the major embodiments provided below. As such, according to some embodiments, the CD3 zeta comprises an amino acid sequence that has at least 70%, such as 80%, 85%, 90%, 92%, 94%, 96%, 98%, or 100%, sequence identity to SEQ ID NO: 1.

In some embodiments, the engineered CD3z subunit includes a co-stimulatory region (e.g., any of the co-stimulatory regions described herein) operably linked or fused to a CD3z component (e.g., any of the CD3z components described herein). In some embodiments, the CD3z component described herein includes a human CD3z, a functional portion, or a functional variant thereof. For example, the CD3z component can be a full-length human CD3z. In some embodiments, the CD37z component described herein comprises an amino acid sequence that has at least 70%, such as 80%, 85%, 90%, 92%, 94%, 96%, 98%, or 100%, sequence identity to SEQ ID NO: 1. In some embodiments, the CD3z component described herein includes an intracellular domain, and the co-stimulatory region described herein is within the intracellular domain of the CD3z component described herein.

In some embodiments, the engineered CD3 zeta subunit is configured such that at least one of the following effects is realized: (1) the immune cell expressing the engineered CD3 zeta subunit has a reduced activation in the absence of antigen stimulation compared with when the immune cell does not express the engineered CD3 zeta subunit; (2) the immune cell expressing the engineered CD3 zeta subunit has a reduced cytotoxicity against non-target cells compared with when the immune cell does not express the engineered CD3 zeta subunit; (3) the immune cell expressing the engineered CD3 zeta subunit has an increased activation upon stimulation compared with when the immune cell does not express the engineered CD3 zeta subunit; or (4) the immune cell expressing the engineered CD3 zeta subunit has an increased immune cell response against target cells corresponding thereto compared with when the immune cell does not express the engineered CD3 zeta subunit.

As used herein, the term “co-stimulatory domain”, or “co-stimulatory signaling domain”, is referred to as a specific functional portion of the engineered CD3 zeta subunit that is capable of recruiting certain intracellular signaling molecules to thereby confer the immune cell at least one of the following capabilities including cytotoxicity, stemness (i.e. the capability to resist exhaustion), memory, persistence, etc. For example, the 4-1BB co-stimulatory domain contains binding motifs for, and therefore is capable of recruiting, tumor necrosis factor receptor-associated factors (TRAF) signaling adaptor proteins, thereby leading to increased T cell memory and persistence. In another example, the CD28 co-stimulatory domain contains binding motifs for, and thus is capable of recruiting, certain downstream signaling molecules such as phosphatidylinositol-3-kinase (PI3K), growth factor receptor-bound protein 2 (Grb2), and lymphocyte-specific protein tyrosinekinase (Lck), thereby leading to more effective T cell killing but reduced long-term T cell persistence.

Herein, the co-stimulatory domain can be from a natural co-stimulatory immune receptor such as CD28, 4-1BB, OX40, CD2, CD27, CDS, ICAM-1, LFA-1, and ICOS, etc. In some embodiments of the engineered CD3 zeta subunit, the co-stimulatory region comprises a co-stimulatory domain, or a functional portion or a functional variant thereof, of CD28, and as such, the first co-stimulatory region may comprise an amino acid sequence that has at least 70%, such as 80%, 85%, 90%, 92%, 94%, 96%, 98%, or 100%, sequence identity to SEQ ID NO: 2. In some embodiments of the engineered CD3 zeta subunit, the at least one co-stimulatory region comprises a first co-stimulatory region, which comprises a co-stimulatory domain, or a functional portion or a functional variant thereof, of 4-1BB, and as such, the first co-stimulatory region may comprise an amino acid sequence that has at least 70%, such as 80%, 85%, 90%, 92%, 94%, 96%, 98%, or 100%, sequence identity to SEQ ID NO: 3. In other embodiments, the engineered CD3 zeta subunit, the at least one co-stimulatory region comprises a first co-stimulatory region, which comprises a co-stimulatory domain, or a functional portion or a functional variant thereof, of any one of the receptors, OX40, CD2, CD27, CDS, ICAM-1, LFA-1, and ICOS. It is noted that the co-stimulatory domain may be from a non-natural source (i.e. artificially created or synthesized) and may contain engineered binding motifs for certain intracellular signaling molecules, which may be a combination of different binding motifs from different co-stimulatory domains of different immune receptors. Such sequences have been reported in K. G. Daniels et al., Science 10.1126/science.abq0225 (2022) and in WO2022173703A1, each of which is incorporated by reference in its entirety.

In some embodiments, the functional portion of a co-stimulatory domain is a portion of the co-stimulatory domain that may wholly or partially contain the functionalities of the co-stimulatory domain. For example, the functional portion may only contain one or more binding motifs for certain downstream signaling molecules of a known co-stimulatory domain. The term “functional variant” is referred to as a sequence variant of the co-stimulatory domain, such as those containing sequence substitutions, deletions, insertions, transpositions, etc., yet the functionalities of the co-stimulatory domain are wholly or partially retained.

In some embodiments, in the engineered CD3 zeta subunit provided herein, the at least one co-stimulatory region may have different locations relative to the CD3 zeta sequence, esp, the intracellular signaling domain of the CD3 zeta.

As used herein, the term “intracellular signaling region of the CD3 zeta” refers to a portion of the intracellular domain of the CD3 zeta subunit that is responsible for transducing the signal upon stimulation of the TCR complex, which typically include the three immunoreceptor tyrosine activation motifs (ITAMs). According to some embodiments, one or more of the at least one co-stimulatory region is fused over the C-terminal of the intracellular signaling domain of the CD3 zeta. Yet according to some other embodiments, one or more of the at least one co-stimulatory region is between a transmembrane domain and the intracellular signaling domain of the CD3 zeta. Yet according to some other embodiments, a first subset of the at least one co-stimulatory region is fused over the C-terminal of the intracellular signaling domain of the CD3 zeta, and a second sub-set of the at least one co-stimulatory region is between a transmembrane domain and the intracellular signaling domain of the CD3 zeta.

According to some specific embodiments, the engineered CD3 zeta subunit comprises a CD28 co-stimulatory region fused to the C-terminal of the CD3 zeta. The CD28 co-stimulatory region comprises an amino acid sequence as set forth in SEQ ID NO: 2, and the CD3 zeta comprises an amino acid sequence as set forth in SEQ ID NO: 1.

According to some specific embodiments, the engineered CD3 zeta subunit comprises a 4-1BB intracellular domain fused to the C-terminal of the CD3 zeta. The 4-1BB intracellular domain comprises an amino acid sequence as set forth in SEQ ID NO: 3, and the CD3 zeta comprises an amino acid sequence as set forth in SEQ ID NO: 1.

There is no limitation to the number of the at least one co-stimulatory region in the engineered CD3 zeta subunit disclosed herein. For example, according to some embodiment, there is only one co-stimulatory region in the engineered CD3 zeta subunit, whereas according to some other embodiments, there are more than one co-stimulatory region in the engineered CD3 zeta subunit. In the latter case, the more than one co-stimulatory region may be from the same immune receptor, or from different immune receptors.

One illustrating example for the engineered CD3 zeta subunit is provided, where the engineered subunit comprises only one co-stimulatory region (or co-stimulatory region) in the intracellular domain thereof. With reference to FIG. 2A, the co-stimulatory region is substantially located at the C-terminus of the engineered CD3 zeta subunit, i.e. the co-stimulatory region is fused to the C-terminus of the CD3 zeta subunit (SEQ ID NO: 1) via a flexible linker (shown as the connecting line between the two blocks representing the CD3z and the co-stimulatory region). Herein, the flexible linker is optional and may be dispensable in some embodiments.

There is no limitation to the actual location of the co-stimulatory region within the engineered CD3z subunit, as long as the CD3z functionality transducing the TCR signaling upon the specific antigen/MHC recognition by the engineered TCR complex. For example, the co-stimulatory region may be located between the transmembrane domain and the immunoreceptor tyrosine activation domain that contains three immunoreceptor tyrosine activation motifs (ITAMs) of the CD3z subunit.

In some embodiments, the co-stimulatory region is an intracellular signaling region, or a functional portion or functional variant thereof of a cell surface protein expressed in the T cells having a co-stimulatory functionality, i.e., being capable of providing co-stimulatory signals for the activation, survival, and/or proliferation of the T cells. Non-limiting examples of a co-stimulatory protein whose intracellular signaling domain or a functional portion thereof (e.g. a functional portion may comprise one or more signaling motifs) that can be used for engineering CD3 zeta subunit may include CD28, 4-1BB, LFA-1, CD4, C CD28, CD27, ICOS, HVEM, LIGHT, CD40L, 4-1BB, OX40, DR3, GITR, CD30, TIM1, SLAM, CD2, CD226.

In some embodiments, the co-stimulatory region can be from CD28 or 4-1BB, and can optionally have an amino acid sequence with 80-100% sequence identity to SEQ ID NOS: 2 and 3, respectively. In some embodiments, the co-stimulatory region can optionally be fused to the CD3z without any linker, or optionally through a flexible linker having a length of 1-20 amino acid residues and comprising largely less bulky amino acid residues such as Glycine (“G”) or Serine (“S”). According to one specific embodiment, the flexible linker can have a sequence as set forth in SEQ ID NO: 6 or SEQ ID NO: 7.

In some embodiments, the co-stimulatory region described herein is located between a transmembrane domain and an intracellular domain of the CD3z component described herein. In some embodiments, the co-stimulatory region is a STAT binding region, e.g., STAT3 or STAT5 binding region. In some embodiments, the STAT3 binding region may comprise an amino acid sequence that has at least 50%, 75% or 100% sequence identity to YXXQ (SEQ ID NO: 22), where the two X residues can be the same or different. In some embodiments, the first X residue is R (or any amino acids with similar physical and/or chemical properties, e.g., H or K) and the second residue is H (or any amino acids with similar physical and/or chemical properties, e.g., R or K). In some embodiments, the STAT3 binding region may comprise an amino acid sequence that has at least 50%, 75% or 100% sequence identity to YRHQ (SEQ ID NO: 21). In some embodiments, the STAT5 binding region may comprise an amino acid sequence that has at least 50%, 75% or 100% sequence identity to YXXL (SEQ ID NO: 23), where the two X residues can be the same or different. In some embodiments, the first X residue is L (or any amino acids with similar physical and/or chemical properties, e.g., A, V, I, M, F, Y, or W) and the second residue is S (or any amino acids with similar physical and/or chemical properties, e.g., T, N, or Q). In some embodiments, the STAT5 binding region may comprise an amino acid sequence that has at least 50%, 75% or 100% sequence identity to YLSL (SEQ ID NO: 24). In some embodiments, the STAT binding region is located in a region of the CD3z component corresponding to positions 150-164 of SEQ ID NO: 1, e.g., between any two amino acid residues corresponding to positions 149, 150, 151, 152, 153, 154, 155, 156, 157, 158, 159, 160, 161, 162, 163, 164, and 165 of SEQ ID NO: 1. In some embodiments, the sequence corresponding to a wildtype human CD3z between the two residues described above is deleted. In some embodiments, the STAT binding region is inserted to the CD3z component (e.g., any of the CD3z components described herein), within an intracellular domain thereof. In some embodiments, the STAT binding region is inserted at a location of the CD3z component corresponding to position 157 and position 158 of SEQ ID NO: 1. In some embodiments, the CD3z component, e.g., after insertion of the STAT binding region (e.g., any of the STAT3 or STAT5 binding regions described herein), may comprise an amino acid sequence that has at least 70%, such as 80%, 85%, 90%, 92%, 94%, 96%, 98%, or 100%, sequence identity to SEQ ID NO: 20 (when a STAT3 binding region YRHQ is inserted) or SEQ ID NO: 29 (when a STAT5 binding region YLSL is inserted).

In some embodiments, one or more co-stimulatory regions are fused to the C-terminus of the CD3 zeta component. In some embodiments, one or more co-stimulatory regions are inserted between a transmembrane domain and an intracellular domain of the CD3 zeta component. In some embodiments, the engineered CD3 zeta subunit comprises one, two, three, or four co-stimulatory regions. In some embodiments, the one or more co-stimulatory regions have the same sequence. In some embodiments, the one or more co-stimulatory regions are derived from the same co-stimulatory receptor (e.g., 4-1BB). In some embodiments, the one or more co-stimulatory regions are derived from different co-stimulatory receptors (e.g., 4-1BB and CD28).

In some embodiments, the co-stimulatory region comprises the amino acid sequence set forth in SEQ ID NO: 2 (RSKRSRLLHSDYMNMTPRRPGPTRKHYQPYAPPRDFAAYRS). It is to be noted that the underlined 4-AA portion in SEQ ID NO: 2 is the well-acknowledged core functional co-stimulatory sequence, yet unexpectedly, we found that the addition of the C-terminal 28-AA segment that is enriched with positively charged amino acids can improve the phenotypes by reducing the tonic signaling of Aspire-TCR.

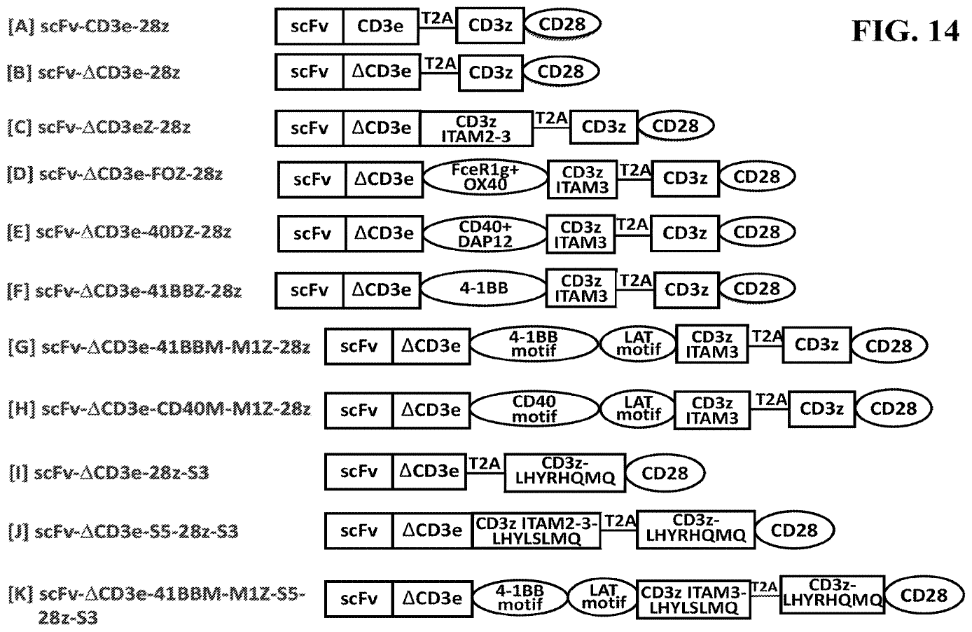

In some embodiments, provided herein is an engineered CD37z subunit, including from N-terminus to C-terminus, a human CD3z and a CD28 co-stimulatory domain. In some embodiments, the engineered CD3z submit is encoded by any one of the constructs shown as Designs A-H in FIG. 14. In some embodiments, the human CD3z described herein includes an amino acid sequence that has at least 70%, such as 80%, 85%, 90%, 92%, 94%, 96%, 98%, or 100%, sequence identity to SEQ ID NO: 1. In some embodiments, the CD28 co-stimulatory domain described herein includes an amino acid sequence that has at least 70%, such as 80%, 85%, 90%, 92%, 94%, 96%, 98%, or 100%, sequence identity to SEQ ID NO: 2.

In some embodiments, provided herein is an engineered CD3z subunit, including from N-terminus to C-terminus, a modified human CD3z and a CD28 co-stimulatory domain. In some embodiments, the engineered CD3z submit is encoded by any one of the constructs shown as Designs I-K in FIG. 14. In some embodiments, the modified human CD3z includes a STAT binding region (e.g., a STAT3 or STAT5 binding region) inserted within its intracellular region (e.g., between a location corresponding to position 157 and position 158 of SEQ ID NO: 1). In some embodiments, a STAT3 binding region (e.g., SEQ ID NO: 22 or SEQ ID NO: 21) is used for insertion herein. In some embodiments, the modified human CD3z described herein includes an amino acid sequence that has at least 70%, such as 80%, 85%, 90%, 92%, 94%, 96%, 98%, or 100%, sequence identity to SEQ ID NO: 20. In some embodiments, the CD28 co-stimulatory domain described herein includes an amino acid sequence that has at least 70%, such as 80%, 85%, 90%, 92%, 94%, 96%, 98%, or 100%, sequence identity to SEQ ID NO: 2. In some embodiments, the CD3z component described herein consists of or comprises the modified human CD3z described herein.

### Engineered Target-Recognizing Subunit

In one aspect, the present disclosure further provides an engineered target-recognizing subunit which, when expressed in an immune cell, can confer the specific targeting of the immune cell against the target cells expressing the specific target molecules recognizable by the engineered target-recognizing subunit.

The engineered target-recognizing subunit comprises an extracellular domain, which comprises a target-recognizing region. As used herein, the term “target-recognizing region” is exchangeable to “target-recognizing moiety” or “target-recognizing portion” or alike, and is referred to as a portion of the engineered target-recognizing subunit within the extracellular domain thereof that can specifically recognize and bind to a target molecule (i.e. cognate binding partner) that is expressed on the target cells to thereby allow the engineered TCR complex to exert its cytotoxicity against the target cells.

In some embodiments, the target-recognizing region may comprise a single-chain variable fragment (scFv) or a single-domain antibody (sdAb or nanobody) that can specifically recognize and bind to an epitope of an antigen presented on the surface of the target cells that corresponds to the scFv or the sdAb (i.e. the target molecules). In some embodiments, any scFv or sdAb that can specifically target the target cell-enriched target molecules can be used as the target-recognizing region of the engineered TCR complex.

In some embodiments, the target-recognizing region may comprise a ligand, or a functional portion or a functional variant thereof (herein, “functional” is defined as being capable of binding to the corresponding receptor), that can specifically recognize and bind to a cell-surface receptor corresponding thereto that is expressed on the surface of the target cells. Under this scenario, any ligand, or a receptor binding portion or a functional variant thereof, that can specifically target the target cell-enriched target molecules can be used as the target-recognizing region of the engineered target-recognizing subunit. Herein the ligand or a functional portion or variant thereof may be a natural ligand, but may also be a peptide that has been artificially identified or engineered.

In some embodiments, the engineered target-recognizing subunit that carries the aforementioned target-recognizing region may be fused to the backbone of any one of TCRα, TCRb, CD3g, CD3d, CD3e, or optionally be based on an engineered protein that can be incorporated into the TCR complex. In some embodiments, the target-recognizing region can be in any location of the engineered target-recognizing subunit, as long as it is within the extracellular domain thereof. For example, the target-recognizing region can be located over the N-terminus of one of the above mentioned five subunits (i.e., TCRa, TCRb, CD3g, CD3d or CD3e), with FIG. 2B illustrating one embodiment where the target-recognizing region (ligand) is fused with the CD3e (either full-length CD3e or a truncated form) via a GS-rich linker (i.e. “GS linker”).

In some embodiments, the engineered target-recognizing subunit comprises a CD3e. In some embodiments, the CD3e comprises an amino acid sequence that has at least 70%, such as 80%, 85%, 90%, 92%, 94%, 96%, 98%, or 100%, sequence identity to SEQ ID NO: 5.

In some embodiments, the target-recognizing region comprises an IL13(E13Y) ligand region (“IL13E13Y”). In some embodiments, the target-recognizing region comprises an amino acid sequence that has at least 70%, such as 80%, 85%, 90%, 92%, 94%, 96%, 98%, or 100%, sequence identity to SEQ ID NO: 4. In some embodiments, the target-recognizing region comprises an anti-ALPP (“F8”) scFv. In some embodiments, the target-recognizing region comprises an amino acid sequence that has at least 70%, such as 80%, 85%, 90%, 92%, 94%, 96%, 98%, or 100%, sequence identity to SEQ ID NO: 9.

In some embodiments, the engineered target-recognizing subunit comprises a CD3e that is fused to the target-recognizing region via a linker (e.g., a GS-linker). In some embodiments, the linker comprises an amino acid sequence that has at least 70%, such as 80%, 85%, 90%, 92%, 94%, 96%, 98%, or 100%, sequence identity to SEQ ID NO: 6 or SEQ ID NO: 7.

Herein, the engineered protein (i.e. the backbone protein based on which the target-recognizing region is fused to or incorporated into) can comprise an extracellular domain that harbors the target-recognizing region, a transmembrane domain, and optionally an intracellular domain. Each of these domains may be from any of the TCR-CD3 complex subunits such as TCRa, TCRb, CD3g, CD3d, CD3e, and CD3z, or their combinations, or may be artificially engineered.

In some examples of the backbone protein, the extracellular domain described herein may comprise the extracellular domain of the CD3e subunit which is further fused to the target-recognizing region to its N-terminus in the engineered target-recognizing subunit, the transmembrane domain described herein may comprise the transmembrane domain of the CD3e subunit, and the intracellular domain described herein may comprise the intracellular domain of the CD3z subunit.

In another example, the extracellular domain described herein may comprise the extracellular domain of the CD3g subunit which is further fused to the target-recognizing region over its N-terminus, the transmembrane domain described herein may comprise the transmembrane domain of the CD3e subunit, and the intracellular domain described herein may comprise the intracellular domain of the CD3z subunit.

In yet another example, the extracellular domain described herein may comprise the extracellular domain of the TCRa subunit which is further fused to the target-recognizing region over its N-terminus, the transmembrane domain described herein may comprise the transmembrane domain of the CD3e subunit, and the intracellular domain described herein may comprise the intracellular stimulating domain of the CD3z subunit fused with the intracellular stimulating domain of the CD3e subunit.

It is noted that the engineered proteins described above are only illustrating examples, and further engineering can be made to each of the extracellular domain. For example, the intracellular domain may comprise 0-10 (e.g., 0, 1, 2, 3, 4, 5, 6, 7, 8, 9, or 10) intracellular signaling motifs (e.g. ITAMs), each from the CD3e, CD3g, CD3g, and CD3z, or as an engineered ITAM.

In some embodiments, the engineered target-recognizing subunit comprises at least one functional region in an intracellular domain thereof. In some embodiments, the at least one functional region is fused to the C-terminus of the CD3 epsilon component.

In some embodiments, the at least one functional region comprises an immune receptor tyrosine based activation motif (ITAM). In some embodiments, the ITAM is derived from CD3z, CD3g, DAP12, FcαRI, FcγRI, FcγRII, FcγRIII, Dectin-1, CLEC-1, CD28, or CD72. In some embodiments, the ITAM comprises: (1) a CD3z ITAM3 that comprises an amino acid sequence that is at least 70%, 80%, 85%, 90%, 95%, or 100% identical to the amino acid sequence of SEQ ID NO: 11; or (2) a CD3z ITAM2-3 that comprises an amino acid sequence that is at least 70%, 80%, 85%, 90%, 95%, or 100% identical to the amino acid sequence of SEQ ID NO: 12. In some embodiments, the ITAM is located at the C-terminus of the engineered CD3 epsilon subunit. In some embodiments, the ITAM is fused to the C-terminus of the CD3 epsilon component, wherein the ITAM comprises a CD3z ITAM3 or a CD3z ITAM2-3. In some embodiments, the ITAM comprises a STAT binding region, wherein the STAT binding region is a STAT3 binding motif (e.g., any of the STAT3 binding motifs described herein) or a STAT5 binding motif (e.g., any of the STAT5 binding motifs described herein).

In some embodiments, the at least one functional region comprises any one or a combination of: (1) an FcεR1γ intracellular domain, which comprises an amino acid sequence that is at least 90%, 95%, or 100% identical to the amino acid sequence of SEQ ID NO: 25; (2) an OX40 intracellular domain, which comprises an amino acid sequence that is at least 90%, 95%, or 100% identical to the amino acid sequence of SEQ ID NO: 26; (3) a CD40 intracellular domain, which comprises an amino acid sequence that is at least 90%, 95%, or 100% identical to the amino acid sequence of SEQ ID NO: 27; (4) a DAP12 intracellular domain, which comprises an amino acid sequence that is at least 90%, 95%, or 100% identical to the amino acid sequence of SEQ ID NO: 28; (5) a 4-1BB intracellular domain, which comprises an amino acid sequence that is at least 90%, 95%, or 100% identical to the amino acid sequence of SEQ ID NO: 3; (6) a 4-1BB motif, which comprises an amino acid sequence that is at least 90%, 95%, or 100% identical to the amino acid sequence of SEQ ID NO: 15; (7) a CD40 motif, which comprises an amino acid sequence that is at least 90%, 95%, or 100% identical to the amino acid sequence of SEQ ID NO: 17; and (8) a linker for activation of T cells (LAT) motif, which comprises an amino acid sequence that is at least 90%, 95%, or 100% identical to the amino acid sequence of SEQ ID NO: 16.

In some embodiments, the engineered CD3 epsilon subunit comprises a STAT binding region within an intracellular domain thereof. In some embodiments, the STAT binding region comprises: (1) a STAT3 binding motif, which comprises an amino acid sequence that is at least 50%, 75%, or 100% identical to the amino acid sequence of SEQ ID NO: 22; or (2) a STAT5 binding motif, which comprises an amino acid sequence that is at least 50%, 75%, or 100% identical to the amino acid sequence of SEQ ID NO: 23. In some embodiments, the STAT3 binding region described herein may comprise an amino acid sequence that has at least 50%, 75% or 100% sequence identity to YXXQ (SEQ ID NO: 22), where the two X residues can be the same or different. In some embodiments, the first X residue is R (or any amino acids with similar physical and/or chemical properties, e.g., H or K) and the second residue is H (or any amino acids with similar physical and/or chemical properties, e.g., R or K). In some embodiments, the STAT5 binding region described herein may comprise an amino acid sequence that has at least 50%, 75% or 100% sequence identity to YXXL (SEQ ID NO: 23), where the two X residues can be the same or different. In some embodiments, the first X residue is L (or any amino acids with similar physical and/or chemical properties, e.g., A, V, I, M, F, Y, or W) and the second residue is S (or any amino acids with similar physical and/or chemical properties, e.g., T, N, or Q). In some embodiments, the STAT binding region comprises a STAT3 binding motif, which comprises an amino acid sequence that is at least 50%, 75%, or 100% identical to the amino acid sequence of SEQ ID NO: 21. In some embodiments, the STAT binding region comprises a STAT5 binding motif, which comprises an amino acid sequence that is at least 50%, 75%, or 100% identical to the amino acid sequence of SEQ ID NO: 24.

In some embodiments, the engineered CD3 epsilon subunit described herein comprises an ITAM in the intracellular domain thereof, wherein ITAM comprises a STAT binding region (e.g., any of the STAT binding regions described herein). In some embodiments, the ITAM is a CD32 ITAM2-3 motif, which comprises an amino acid sequence that is at least 70%, 80%, 85%, 90%, 95%, or 100% identical to the amino acid sequence of SEQ ID NO: 18 (when a STAT5 binding region YLSL is inserted). In some embodiments, the ITAM is a CD3z ITAM3 motif, which comprises an amino acid sequence that is at least 70%, 80%, 85%, 90%, 95%, or 100% identical to the amino acid sequence of SEQ ID NO: 19 (when a STAT5 binding region YLSL is inserted). In some embodiments, the ITAM described herein can include a STAT3 binding region (e.g., any of the STAT3 binding regions described herein) inserted. In some embodiments, the ITAM described herein can include a STAT5 binding region (e.g., any of the STAT5 binding regions described herein) inserted. In some embodiments, the insertion site can be between any two residues within the CD3z ITAM2-3 motif or the CD3z ITAM3 motif described herein. In some embodiments, the insertion site corresponds to position 58 and position 59 of a CD3z ITAM2-3 motif (SEQ ID NO: 12). In some embodiments, the insertion site corresponds to position 27 and position 28 of a CD3z ITAM3 motif (SEQ ID NO: 11). In some embodiments, the at least one functional region comprises both an FcεR1γ intracellular domain and an OX40 intracellular domain. In some embodiments, the at least one functional region comprises a first compound functional region comprising, from an N-terminus to a C-terminus direction, the FcεR1γ intracellular domain and the OX40 intracellular domain, wherein the first compound functional region comprises an amino acid sequence that is at least 70%, 80%, 85%, 90%, 95%, or 100% identical to the amino acid sequence of SEQ ID NO: 13. In some embodiments, the at least one functional region further comprises, over the C-terminus of the first compound functional region, an ITAM, wherein the ITAM comprises CD3z ITAM3 or CD3z ITAM2-3. In some embodiments, the at least one functional region comprises both a CD40 intracellular domain and a DAP12 intracellular domain. In some embodiments, the at least one functional region comprises a second compound functional region comprising, from an N-terminus to a C-terminus direction, the CD40 intracellular domain and the DAP12 intracellular domain, wherein the second compound functional region comprises an amino acid sequence that is at least 90%, 95%, or 100% identical to the amino acid sequence of SEQ ID NO: 14. In some embodiments, the at least one functional region further comprises, at the C-terminus of the second compound functional region, an ITAM, wherein the ITAM comprises a CD3z ITAM3 or a CD3z ITAM2-3. In some embodiments, the engineered CD3 epsilon subunit comprises, from the N-terminus to the C-terminus thereof, a 4-1BB intracellular domain and an ITAM, wherein the ITAM comprises a CD3z ITAM3 or a CD3z ITAM2-3. In some embodiments, the at least one functional region comprises both a 4-1BB motif and an LAT motif.

In some embodiments, the engineered CD3 epsilon subunit comprises an ITAM at the C-terminus of the engineered CD3 epsilon subunit. In some embodiments, the ITAM comprises a CD3z ITAM3 or a CD3z ITAM2-3. In some embodiments, the ITAM comprises a STAT binding region, wherein the STAT binding region is a STAT3 binding motif or a STAT5 binding motif.

In some embodiments, the at least one functional region comprises both a CD40 motif and an LAT motif. In some embodiments, the engineered CD3 epsilon subunit comprises an ITAM at the C-terminus of the engineered CD3 epsilon subunit. In some embodiments, the ITAM comprises a CD3z ITAM3 or a CD3z ITAM2-3. In some embodiments, the target-recognizing region is operably linked to or incorporated into the CD3 epsilon component.

In some embodiments, the target-recognizing region comprises: (1) an antigen-binding region; or (2) a ligand or a fragment thereof that binds to a cell-surface receptor expressed on a target cell of the immune cell. In some embodiments, the antigen-binding region comprises a single-chain variable fragment (scFv) that specifically recognizes ALPP, LYPD3, IL13Ra2, BCMA, CD16, CD19, CD20, CD22, CD27 CD138, CD33, CD123, CD171, CD70, CD7, CS-1, PSMA, PSCA, ROR1, GD2, MUC1, MUC16, HER2(ErbB2), MET, EphA2, EpCAM, CEA, CSPG4, Lewis Y antigen, Mesothelin, NKG2D, Glypican-3 (GPC-3), FAP, FRa (folate receptoralpha), EGFR, EGFR vIII, IL-11Ra (IL11 receptor alpha), VEGFR-II, B7-H6, and DNAM-1. In some embodiments, the antigen-binding region comprises a single-chain variable fragment (scFv) against ALPP. In some embodiments, the antigen-binding region comprises a single-domain antibody (sdAb or nanobody). In some embodiments, the ligand is selected from the group consisting of IL13(E13Y), IL-13, IL-11, IL-10, APRIL, GM-CSF, TPO, Adnectin, T1E, FLT3L, EPHRIN B2, CTLX, LFA-1, and FSH. In some embodiments, the ligand is IL13(E13Y).

In some embodiments, provided herein is an engineered target-recognizing subunit (e.g., any of the CD3e submits described herein).

In some embodiments, provided herein is an engineered CD3e subunit, including from N-terminus to C-terminus, a target recognizing region (e.g., any of the target recognizing regions described herein), and a full-length human CD3e. In some embodiments, the engineered CD3e subunit is encoded by the construct shown as Design A in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the human CD3e described herein includes an amino acid sequence that has at least 70%, such as 80%, 85%, 90%, 92%, 94%, 96%, 98%, or 100%, sequence identity to SEQ ID NO: 5.

In some embodiments, provided herein is an engineered CD3e subunit, including from N-terminus to C-terminus, a target recognizing region (e.g., any of the target recognizing regions described herein), and a truncated human CD3e. In some embodiments, the engineered CD3e subunit is encoded by any one of the constructs shown as Design B and Design I in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10.

In some embodiments, provided herein is an engineered CD3e subunit, including from N-terminus to C-terminus, a target recognizing region (e.g., any of the target recognizing regions described herein), a truncated human CD3e, and a CD3z ITAM2-3 (e.g., any of the CD3z ITAM2-3 motifs described herein). In some embodiments, the engineered CD3e subunit is encoded by the construct shown as Design C in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the CD3z ITAM2-3 includes an amino acid sequence that has at least 70%, such as 80%, 85%, 90%, 92%, 94%, 96%, 98%, or 100%, sequence identity to SEQ ID NO: 12.

In some embodiments, provided herein is an engineered CD3e subunit, including from N-terminus to C-terminus, a target recognizing region (e.g., any of the target recognizing regions described herein), a truncated human CD3e, a FcεR1γ intracellular domain (e.g., any of the FcεR1γ intracellular domains described herein), an OX40 intracellular domain (e.g., any of the OX40 intracellular domains described herein), and a CD3z ITAM3 (e.g., any of the CD3z ITAM3 motifs described herein). In some embodiments, the engineered CD3e subunit is encoded by the construct shown as Design D in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the FcεR1γ intracellular domain and the OX40 intracellular domain together include an amino acid sequence that has at least 70%, such as 80%, 85%, 90%, 92%, 94%, 96%, 98%, or 100%, sequence identity to SEQ ID NO: 13. In some embodiments, the CD3z ITAM3 includes an amino acid sequence that has at least 70%, such as 80%, 85%, 90%, 92%, 94%, 96%, 98%, or 100%, sequence identity to SEQ ID NO: 11.

In some embodiments, provided herein is an engineered CD3e subunit, including from N-terminus to C-terminus, a target recognizing region (e.g., any of the target recognizing regions described herein), a truncated human CD3e, a CD40 intracellular domain (e.g., any of the CD40 intracellular domains described herein), an DAP12 intracellular domain (e.g., any of the DAP12 intracellular domains described herein), and a CD3z ITAM3 (e.g., any of the CD3z ITAM3 motifs described herein). In some embodiments, the engineered CD3e subunit is encoded by the construct shown as Design E in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the CD40 intracellular domain and the DAP12 intracellular domain together include an amino acid sequence that has at least 70%, such as 80%, 85%, 90%, 92%, 94%, 96%, 98%, or 100%, sequence identity to SEQ ID NO: 14. In some embodiments, the CD3z ITAM3 includes an amino acid sequence that has at least 70%, such as 80%, 85%, 90%, 92%, 94%, 96%, 98%, or 100%, sequence identity to SEQ ID NO: 11.

In some embodiments, provided herein is an engineered CD3e subunit, including from N-terminus to C-terminus, a target recognizing region (e.g., any of the target recognizing regions described herein), a truncated human CD3e, a 4-1BB intracellular domain (e.g., any of the 4-1BB intracellular domains described herein), and a CD3z ITAM3 (e.g., any of the CD3z ITAM3 motifs described herein). In some embodiments, the engineered CD3e subunit is encoded by the construct shown as Design F in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the 4-1BB intracellular domain includes an amino acid sequence that has at least 70%, such as 80%, 85%, 90%, 92%, 94%, 96%, 98%, or 100%, sequence identity to SEQ ID NO: 3. In some embodiments, the CD3z ITAM3 includes an amino acid sequence that has at least 70%, such as 80%, 85%, 90%, 92%, 94%, 96%, 98%, or 100%, sequence identity to SEQ ID NO: 11.

In some embodiments, provided herein is an engineered CD3e subunit, including from N-terminus to C-terminus, a target recognizing region (e.g., any of the target recognizing regions described herein), a truncated human CD3e, a 4-1BB motif (e.g., any of the 4-1BB motifs described herein), a LAT motif (e.g., any of the LAT motifs described herein), and a CD3z ITAM3 (e.g., any of the CD3z ITAM3 motifs described herein). In some embodiments, the engineered CD3e subunit is encoded by the construct shown as Design G in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the 4-1BB motif includes an amino acid sequence that has at least 70%, such as 80%, 85%, 90%, 92%, 94%, 96%, 98%, or 100%, sequence identity to SEQ ID NO: 15. In some embodiments, the LAT motif includes an amino acid sequence that has at least 70%, such as 80%, 85%, 90%, 92%, 94%, 96%, 98%, or 100%, sequence identity to SEQ ID NO: 16. In some embodiments, the CD3z ITAM3 includes an amino acid sequence that has at least 70%, such as 80%, 85%, 90%, 92%, 94%, 96%, 98%, or 100%, sequence identity to SEQ ID NO: 11.

In some embodiments, provided herein is an engineered CD3e subunit, including from N-terminus to C-terminus, a target recognizing region (e.g., any of the target recognizing regions described herein), a truncated human CD3e, a CD40 motif (e.g., any of the CD40 motifs described herein), a LAT motif (e.g., any of the LAT motifs described herein), and a CD3z ITAM3 (e.g., any of the CD3z ITAM3 motifs described herein). In some embodiments, the engineered CD3e subunit is encoded by the construct shown as Design H in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the CD40 motif includes an amino acid sequence that has at least 70%, such as 80%, 85%, 90%, 92%, 94%, 96%, 98%, or 100%, sequence identity to SEQ ID NO: 17. In some embodiments, the LAT motif includes an amino acid sequence that has at least 70%, such as 80%, 85%, 90%, 92%, 94%, 96%, 98%, or 100%, sequence identity to SEQ ID NO: 16. In some embodiments, the CD3z ITAM3 includes an amino acid sequence that has at least 70%, such as 80%, 85%, 90%, 92%, 94%, 96%, 98%, or 100%, sequence identity to SEQ ID NO: 11.

In some embodiments, provided herein is an engineered CD3e subunit, including from N-terminus to C-terminus, a target recognizing region (e.g., any of the target recognizing regions described herein), a truncated human CD3e, and a CD3z ITAM2-3 (e.g., any of the CD3z ITAM2-3 motifs described herein with a STAT5 binding region). In some embodiments, the engineered CD3e subunit is encoded by the construct shown as Design J in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the CD3z ITAM2-3 (with a STAT5 binding region) includes an amino acid sequence that has at least 70%, such as 80%, 85%, 90%, 92%, 94%, 96%, 98%, or 100%, sequence identity to SEQ ID NO: 18.

In some embodiments, provided herein is an engineered CD3e subunit, including from N-terminus to C-terminus, a target recognizing region (e.g., any of the target recognizing regions described herein), a truncated human CD3e, a 4-1BB motif (e.g., any of the 4-1BB motifs described herein), a LAT motif (e.g., any of the LAT motifs described herein), and a CD3z ITAM3 (e.g., any of the CD3z ITAM3 motifs described herein with a STAT5 binding region). In some embodiments, the engineered CD3e subunit is encoded by the construct shown as Design K in FIG. 14. In some embodiments, the target recognizing region is an scFv. In some embodiments, the scFc described herein includes an amino acid sequence that has at least 70%, such as 80%, 85%, 90%, 92%, 94%, 96%, 98%, or 100%, sequence identity to SEQ ID NO: 9 or SEQ ID NO: 30. In some embodiments, the truncated human CD3e described herein does not include an intracellular domain of human CD3e (e.g., a functional intracellular domain of human CD3e). In some embodiments, the truncation (e.g., any modification, deletion, insertion, and/or substitution) is within a region corresponding to position 151 to position 179 of SEQ ID NO: 5. In some embodiments, the truncated human CD3e does not include an amino acid sequence corresponding to positions 151-179 of SEQ ID NO: 5. In some embodiments, the truncated human CD3e described herein includes an amino acid sequence that has at least 70%, such as 80%, 85%, 90%, 92%, 94%, 96%, 98%, or 100%, sequence identity to SEQ ID NO: 10. In some embodiments, the 4-1BB motif includes an amino acid sequence that has at least 70%, such as 80%, 85%, 90%, 92%, 94%, 96%, 98%, or 100%, sequence identity to SEQ ID NO: 15. In some embodiments, the LAT motif includes an amino acid sequence that has at least 70%, such as 80%, 85%, 90%, 92%, 94%, 96%, 98%, or 100%, sequence identity to SEQ ID NO: 16. In some embodiments, the CD3z ITAM3 (with a STAT5 binding region) includes an amino acid sequence that has at least 70%, such as 80%, 85%, 90%, 92%, 94%, 96%, 98%, or 100%, sequence identity to SEQ ID NO: 19.

### Engineered TCR Complex System

In some embodiments, provided herein is an engineered TCR complex system, including an engineered CD3e subunit (e.g., any of the CD3e subunits described herein). In some embodiments, the engineered TCR complex system further includes an engineered CD3z subunit (e.g., any of the CD3z subunits described herein).

In some embodiments, the engineered CD3z subunit described herein includes a co-stimulatory region (e.g., any of the co-stimulatory regions described herein). In some embodiments, the co-stimulatory region described herein is human CD28, a functional portion or a functional variant thereof. In some embodiments, the co-stimulatory region described herein is human 4-1BB, a functional portion or a functional variant thereof.

In some embodiments, one or both of the engineered CD3e subunit and the engineered CD3z subunit described herein includes a STAT binding region (e.g., any of the STAT binding regions described herein). In some embodiments, the STAT binding region described herein is within the intracellular domain of the engineered CD3e subunit and the engineered CD3z subunit described herein. Specifically, in some embodiments, the engineered CD3e subunit described herein includes a STAT5 binding region, and the engineered CD3z subunit described herein includes a STAT3 binding region; in some embodiments, the engineered CD3e subunit described herein includes a STAT3 binding region, and the engineered CD3z subunit described herein includes a STAT5 binding region. In some embodiments, the engineered CD3e subunit described herein includes a STAT5 binding region or a STAT3 binding region, and the engineered CD3z subunit described herein does not include a STAT5 binding region or a STAT3 binding region. In some embodiments, the engineered CD3z subunit described herein includes a STAT5 binding region or a STAT3 binding region, and the engineered CD3e subunit described herein does not include a STAT5 binding region or a STAT3 binding region.

In some embodiments, when the engineered CD3e subunit described herein is expressed in a population of T cells, at least one of the following is met if compared to when the STAT binding region is absent in any of the engineered CD3 epsilon subunit or the engineered CD3 zeta subunit: the population of T cells comprise a higher percentage (e.g., at least 10%, 20%, 30%, 40%, 50%, 60%, 70%, 80%, 90%, 1-fold, 2-fold, 5-fold, 10-fold, 20-fold, 50-fold, or 100-fold) of terminally differentiated effector T cells; the population of T cells exhibit an increased (e.g., at least 10%, 20%, 30%, 40%, 50%, 60%, 70%, 80%, 90%, 1-fold, 2-fold, 5-fold, 10-fold, 20-fold, 50-fold, or 100-fold) antigen-specific stimulation in vitro; and the population of T cells exhibit an increased (e.g., at least 10%, 20%, 30%, 40%, 50%, 60%, 70%, 80%, 90%, 1-fold, 2-fold, 5-fold, 10-fold, 20-fold, 50-fold, or 100-fold) expansion in vitro upon repeated stimulation with the antigen.

In some embodiments, the engineered CD3e subunit and the engineered CD3z subunit described herein can interact with endogenous (e.g., unmodified) TCR subunits, endogenous (e.g., unmodified) CD3g subunits, and/or endogenous (e.g., unmodified) CD3d subunits, forming a functional TCR-CD3 complex.

### Modulating the Activities of an Immune Cell

In one aspect, the disclosure provides a method for modulating activities of an immune cell.

In some embodiments, the method substantially comprises the step of expressing an exogenous CD3 zeta subunit in the immune cell. Herein, the exogenous CD3 zeta subunit can be merely a mammalian CD3 zeta subunit, or a functional portion or a functional variant thereof, or more preferably can be an engineered CD3 zeta subunit according to any one of the embodiments of the engineered CD3 zeta subunit as described above.

In some embodiments, the method is configured such that after the above step is administered to the immune cell, the at least one of the following effects is satisfied:
(1) the immune cell expressing the engineered CD3 zeta subunit has a reduced activation in the absence of antigen stimulation of the TCR complex compared with when the immune cell does not express the engineered CD3 zeta subunit;
(2) the immune cell expressing the engineered CD3 zeta subunit has a reduced cytotoxicity against non-target cells compared with when the immune cell does not express the engineered CD3 zeta subunit;
(3) the immune cell expressing the engineered CD3 zeta subunit has an increased activation upon stimulation of the TCR complex compared with when the immune cell does not express the engineered CD3 zeta subunit; or
(4) the immune cell expressing the engineered CD3 zeta subunit has an increased immune cell response against target cells corresponding thereto compared with when the immune cell does not express the engineered CD3 zeta subunit.

In some embodiments, the immune cell may be a T lymphocyte (i.e. T cell), a tumor-infiltrating lymphocytes (TILs), a natural killer (NK) cell, or an NK T cells.

According to some embodiments of the method, the immune cell expresses an engineered TCR or CAR.

### Engineered Cells

The present disclosure provides engineered cells (e.g., T cells) that comprise the engineered CD3 zeta subunit, the engineered target-recognizing subunit, or the engineered TCR complex described herein. These engineered cells can be used to treat various disorders or disease as described herein (e.g., virus infection, cancers, virus-induced disorders).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. More preferably, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a T cell, B cell or NK cell.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. TCR gene-modified T cells can be obtained by transducing T cells (e.g., isolated from the peripheral blood of patients), with a viral vector. In some embodiments, the T cell is a TCR gene-modified T cell. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell.

In some embodiments, the cell is an NK cell. In some embodiments, preparation of the engineered cells includes one or more culture and/or preparation steps. The cells for introduction of the binding molecule, e.g., TCR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. TCR, CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

In some embodiments, the engineered TCR complex, when engineered into a human T cell, may compete with endogenous TCR complexes and/or can form mispairings with endogenous TCR complex components (e.g., TCR or CD3), which may, in certain aspects, reduce the engineered TCR complex's signaling, activity, and/or expression, and ultimately result in reduced activity of the engineered cells. The engineered cell can be genetically modified. In some embodiments, the engineered cells can comprise a genetic disruption of a gene encoding an endogenous TCR complex component. In some embodiments, the engineered cells do not express the endogenous TCR complex component.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector can include an additional nucleic acid encoding an inhibitory protein (e.g., a checkpoint inhibitor). In various embodiments, the cell expresses the genetically engineered antigen receptor and the inhibitory protein. In various embodiments, the inhibitory protein is constitutively expressed.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two or three genes separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER. C6® cells; and NSO cells. In some embodiments, a particular eukaryotic host cell is selected based on its ability to make desired post-translational modifications to the binding molecule. For example, in some embodiments, CHO cells produce polypeptides that have a higher level of sialylation than the same polypeptide produced in 293 cells.

In one aspect, the disclosure provides a nucleic acid comprising a polynucleotide encoding a polypeptide comprising:
(1) the engineered CD3 zeta subunit described herein; and/or
(2) the engineered target-recognizing subunit described herein.

In some embodiments, the polypeptide comprises the engineered CD3 zeta subunit described herein and the engineered target-recognizing subunit described herein. In some embodiments, the engineered CD3 zeta subunit described herein and the engineered target-recognizing subunit are joined via a T2A linker.

In one aspect, the disclosure provides a nucleic acid comprising a polynucleotide encoding a polypeptide comprising:
(1) the engineered CD3 zeta subunit described herein; and/or
(2) the engineered target-recognizing subunit described herein.

In some embodiments, the polypeptide comprises the engineered CD3 zeta subunit described herein and the engineered target-recognizing subunit described herein. In some embodiments, the engineered CD3 zeta subunit described herein and the engineered target-recognizing subunit are joined via a T2A linker.

In some embodiments, the engineered target-recognizing subunit comprises one or more, or all of the following (e.g., from N-terminus to C-terminus): an scFv and a CD3e; and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv and a truncated CD3e; and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, and a CD3z ITAM (e.g., CD3z ITAM2-3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, a FceR1g intracellular domain, an OX40 intracellular domain, and a CD3z ITAM (e.g., CD3z ITAM3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, an OX40 intracellular domain, a DAP12 intracellular domain, and a CD3z ITAM (e.g., CD3z ITAM3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, a 4-1BB intracellular domain, and a CD3z ITAM (e.g., CD3z ITAM3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, a 4-1BB motif, a LAT motif, and a CD3z ITAM (e.g., CD3z ITAM3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv, a truncated CD3e, a CD40 intracellular domain, a LAT motif, and a CD3z ITAM (e.g., CD3z ITAM3); and the engineered CD3 zeta subunit comprises one or more, or all of the following (e.g., from N-terminus to C-terminus): a CD3z and a CD28 co-stimulatory domain.

In some embodiments, the engineered target-recognizing subunit comprises one or more, or all of the following (e.g., from N-terminus to C-terminus): an scFv and a truncated CD3e; and the engineered CD3 zeta subunit comprises one or more, or all of the following (e.g., from N-terminus to C-terminus): a CD3z component (e.g., SEQ ID NO: 20) and a CD28 co-stimulatory domain.

In some embodiments, the engineered target-recognizing subunit comprises one or more, or all of the following (e.g., from N-terminus to C-terminus): an scFv, a truncated CD3e, and a CD3z ITAM (e.g., CD3z ITAM2-3); and the engineered CD3 zeta subunit comprises one or more, or all of the following (e.g., from N-terminus to C-terminus): a CD3z component (e.g., SEQ ID NO: 20) and a CD28 co-stimulatory domain.

In some embodiments, the engineered target-recognizing subunit comprises one or more, or all of the following (e.g., from N-terminus to C-terminus): an scFv, a truncated CD3e, a 4-1BB motif, a LAT motif, and a CD3z ITAM (e.g., CD3z ITAM3-LHYLSLMQ); and the engineered CD3 zeta subunit comprises one or more, or all of the following (e.g., from N-terminus to C-terminus): a CD3z component (e.g., SEQ ID NO: 20) and a CD28 co-stimulatory domain.

In one aspect, the disclosure relates to a vector comprising one or more of the nucleic acids as described herein. In one aspect, the disclosure also relates to a vector comprising two of the nucleic acids as described herein.

The term “Linker” (L) or “linker domain” or “linker region” as used herein refer to an oligo- or polypeptide region from about 1 to 100 amino acids in length, which links together any of the domains/regions. Linkers can be composed of flexible residues like glycine and serine so that the adjacent protein domains are free to move relative to one another. Longer linkers can be used when it is desirable to ensure that two adjacent domains do not sterically interfere with one another. Linkers can be cleavable or non-cleavable. Examples of cleavable linkers include 2A linkers (for example P2A, T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. Other linkers will be apparent to those of skill in the art and can be used in the methods described herein.

The present disclosure also provides a nucleic acid sequence comprising a nucleotide sequence encoding any of the engineered TCR complex, engineered CD3 zeta subunit, or engineered target-recognizing subunit (including e.g., functional portions and functional variants thereof, polypeptides, or proteins described herein).

“Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the engineered CD3 zeta subunit and the nucleotide sequence encoding the engineered target-recognizing subunit are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In some embodiments, the engineered CD3 zeta subunit, the engineered target-recognizing subunit, or the engineered TCR complex described herein is encoded by a nucleotide sequence that has been codon-optimized. In certain embodiments, the engineered target-recognizing subunit, or the engineered TCR complex further comprises a signal peptide. In particular embodiments, the engineered CD3 zeta subunit, the engineered target-recognizing subunit, or the engineered TCR complex described herein is isolated or purified or is recombinant. In some of any such embodiments, the engineered CD3 zeta subunit, the engineered target-recognizing subunit, or the engineered TCR complex described herein is recombinant. In some of any such embodiments, the engineered CD3 zeta subunit, the engineered target-recognizing subunit, or the engineered TCR complex described herein is human.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Method for Preparation of Engineered Cells

The present disclosure provides a method or process for manufacturing and using the engineered cells for treatment of pathological diseases or conditions.

The cells for introduction of the engineered TCR complex can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered cells (e.g., engineered T cells) will seek and destroy antigen positive tumor cells. In some embodiments, the nucleic acid construct further includes a sequence encoding an inhibitory protein. In some embodiments, these engineered cells (e.g., engineered T cells) can block PD-1/PD-L1 immunosuppression and strengthen the antitumor immune response. In some embodiments, the method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the methods further involve introducing into the cell one or more agent, wherein each of the one or more agent is independently capable of inducing a genetic disruption of a gene encoding an endogenous TCR complex component. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells may be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

According to various embodiments described herein, the present disclosure provides an immunotherapy against tumors. In some embodiments, the engineered cells (e.g., engineered T cells) recognize a tumor associated antigen. In some embodiments, these engineered cells (e.g., engineered T cells) demonstrate a stronger antitumor response and reduced T cell exhaustion.

The present disclosure provides a method to create a personalized anti-tumor immunotherapy. In some embodiments, the engineered cells can be produced from a patient's blood cells. These engineered cells are then reinfused into the patient as a cellular therapy product. This product can be applied to any patient who has a tumor.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered cells expressing the engineered TCR complex, the engineered CD3 zeta subunit, or the engineered target-recognizing subunit to a subject in need thereof (e.g., a subject having, or identified or diagnosed as having, a cancer).

In some embodiments, the subject has a solid tumor. In some embodiments, the subject has breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer.

In some embodiments, the compositions and methods disclosed herein can be used for treatment of patients at risk for a cancer. Patients with cancer can be identified with various methods known in the art.

Furthermore, the disclosure provides methods for treating infection or infection associated conditions in a subject. In some embodiments, the treatment can halt, slow, retard, or inhibit progression of the disease. These methods generally involve administering a therapeutically effective amount of genetic engineered cells disclosed herein to a subject in need thereof. In some embodiments, the disease or condition treated is an infectious disease or condition, such as, but not limited to, viral, retroviral, bacterial, and protozoal infections, immunodeficiency, Human Papilloma Virus (HPV), Cytomegalovirus (CMV), Epstein-Barr virus (EBV), adenovirus, BK polyomavirus.

As used herein, by an “effective amount” or “therapeutically effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In any of the methods described herein, the engineered cells and, and/or at least one additional therapeutic agent can be administered to the subject at least once a week (e.g., once a week, twice a week, three times a week, four times a week, once a day, twice a day, or three times a day). In some embodiments, at least two different engineered cells (e.g., cells express different binding molecules) are administered in the same composition (e.g., a liquid composition). In some embodiments, engineered cells and at least one additional therapeutic agent are administered in the same composition (e.g., a liquid composition). In some embodiments, engineered cells and the at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, concurrently with, or after administering the engineered cells to the subject.

In some embodiments, one or more additional therapeutic agents can be administered to the subject. The additional therapeutic agent can be a checkpoint inhibitor (CPI). In some embodiments, the checkpoint inhibitor is an inhibitory protein, e.g., an antibody or antigen binding fragment thereof. The checkpoint inhibitor can inhibit or block one or more immune checkpoints, including e.g., PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, VISTA and combinations thereof. In some embodiments, the inhibitory protein blocks PD-1 or PD-L1. In various embodiments, the inhibitory protein comprises an anti-PD-1 scFv. The inhibitory protein is capable of leading to reduced expression of PD-1 or PD-L1 and/or inhibiting upregulation of PD-1 or PD-L1 in T cells in the population and/or physically obstructing the formation of the PD-1/PD-L1 complex and subsequent signal transduction. In some embodiments, the inhibitory protein blocks PD-1. In some embodiments, the additional therapeutic agent is an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CTLA4 antibody (e.g., ipilimumab), an anti-CD20 antibody (e.g., rituximab), an anti-EGFR antibody (e.g., cetuximab), an anti-CD319 antibody (e.g., elotuzumab), or an anti-PD1 antibody (e.g., nivolumab).

In one some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

### Compositions and Formulations

The present disclosure provides compositions (including pharmaceutical and therapeutic compositions) containing the engineered cells, produced by the methods disclosed herein. Also provided are methods, e.g., therapeutic methods for administrating the engineered cells and compositions thereof to subjects, e.g., patients.

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the engineered cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the engineered cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the engineered cells, by multiple bolus administrations of the engineered cells, or by continuous infusion administration of the engineered cells.

The engineered cells and compositions can be administered using standard administration techniques, formulations, and/or devices. Administration of the engineered cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the engineered cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the engineered cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtrationmembranes.

The compositions or pharmaceutical compositions as described herein can be included in a container, pack, or dispenser together with instructions for administration.

### Methods of Administration

Provided are also methods of administering the engineered cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the engineered cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered cells (e.g., engineered T cells).

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, “Cell transfer immunotherapy for metastatic solid cancer—what clinicians need to know.” Nature reviews Clinical oncology 8.10 (2011): 577; Themeli et al. “Generation of tumor-targeted human T lymphocytes from induced pluripotent stem cells for cancer therapy.” Nature biotechnology 31.10 (2013): 928; Tsukahara et al. “CD19 target-engineered T-cells accumulate at tumor lesions in human B-cell lymphoma xenograft mouse models.” Biochemical and biophysical research communications 438.1 (2013): 84-89; Davila et al. “CD19 CAR-targeted T cells induce long-term remission and B Cell Aplasia in an immunocompetent mouse model of B cell acute lymphoblastic leukemia.” PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the engineered cells are derived from a subject, e.g., patient, in need of a treatment and the engineered cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the engineered cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the HLA class or HLA supertype of the subject is identified. In some embodiments, the subject is treated with a cell therapy that can recognize the antigen in the context of the HLA class or HLA supertype.

In some embodiments, the subject has been treated with a therapeutic agent targeting the disease or condition, e.g. the tumor, prior to administration of the engineered cells or composition containing the engineered cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the engineered cells are administered prophylactically, e.g., to reduce the likelihood of or prevent relapse. In some embodiments, the subject has not received prior treatment with another therapeutic agent.

In some embodiments, the engineered cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the engineered cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the engineered cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the engineered cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the engineered cells are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the engineered cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the engineered cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the engineered cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the engineered cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the engineered cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the TCR described here provides improved expression and activity, thereby providing therapeutic effects even at a low effector to target (E:T) ratio.

Optimal response to therapy can depend on the ability of the engineered recombinant receptors such as TCRs, to be consistently and reliably expressed on the surface of the engineered cells and/or bind the target antigen. For example, in some cases, properties of certain recombinant receptors, e.g., TCRs, can affect the expression and/or activity of the recombinant receptor, in some cases when expressed in a cell, such as a human T cell, used in cell therapy. In some contexts, the level of expression of particular recombinant receptors, e.g., TCRs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered cells (e.g., engineered T cells) (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1.

For the prevention or treatment of disease, the appropriate dosage may depend on the type of disease to be treated, the type of cells or recombinant receptors, the severity and course of the disease, whether the engineered cells are administered for preventive or therapeutic purposes, previous therapy, the subject's clinical history and response to the engineered cells, and the discretion of the attending physician. The compositions and cells are in some embodiments suitably administered to the subject at one time or over a series of treatments.

The engineered cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the engineered cells. In some embodiments, it is administered by multiple bolus administrations of the engineered cells, for example, over a period of no more than 3 days, or by continuous infusion administration of the engineered cells.

In some embodiments, the engineered cells are administered as part of a combination treatment, such as simultaneously with or sequentially with, in any order, another therapeutic intervention, such as an antibody or engineered cell or receptor or agent, such as a cytotoxic or therapeutic agent. The engineered cells in some embodiments are co-administered with one or more additional therapeutic agents or in connection with another therapeutic intervention, either simultaneously or sequentially in any order. In some contexts, the engineered cells are co-administered with another therapy sufficiently close in time such that the cell populations enhance the effect of one or more additional therapeutic agents, or vice versa. In some embodiments, the engineered cells are administered prior to the one or more additional therapeutic agents. In some embodiments, the engineered cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the engineered cells, the biological activity of the engineered cell populations in some embodiments is measured, e.g., by any of a number of known methods. Parameters to assess include specific binding of engineered cells (e.g., engineered T cells) to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al. “Construction and pre-clinical evaluation of an anti-CD19 chimeric antigen receptor.” Journal of immunotherapy (Hagerstown, Md.: 1997) 32.7 (2009): 689 and Hermans et al. “The VITAL assay: a versatile fluorometric technique for assessing CTL- and NKT-mediated cytotoxicity against multiple targets in vitro and in vivo.” Journal of immunological methods 285.1 (2004): 25-40. In certain embodiments, the biological activity of the engineered cells is measured by assaying expression and/or secretion of one or more cytokines, such as CD107a, IFNγ, IL-2, and TNF. In some aspects the biological activity is measured by assessing clinical outcome, such as reduction in tumor burden or load.

### EXAMPLES

In the following, multiple examples are provided to further describe the inventions as covered in the various aspects of the disclosure as set forth above. It is to be noted that these examples serve an illustrating purpose only, and shall not be interpreted to limit the scope of the disclosure.

### Example 1. Designs of Several Aspire-TCR Subunits

In this example, several designs for the Aspire-TCR subunit(s) are provided.

### 1.1. Design of Engineered CD3 Zeta Subunits

FIG. 2A illustrates the structure of an engineered CD3 zeta (i.e. CD3z) subunit according to some embodiments of the disclosure, which is constructed substantially through the fusion of a co-stimulatory region (e.g., the co-stimulatory domain of CD28, 4-1BB, etc.) to the C-terminus of a CD3z component (e.g., a human CD3z). A flexible linker (illustrated as a straight line in FIG. 2A) may be arranged between the CD3z component and the co-stimulatory region, but it is optional. It is noted that according to some other embodiments of the engineered CD3z subunits (not shown), the co-stimulatory region may be within the intracellular domain of a CD3z component, and may be, for example, between the transmembrane domain and the intracellular domain of the CD3z component. Such engineered CD3z subunits may be ectopically expressed in an immune cell so as to improve certain characteristics of the immune cell, such as enhancing the expansion capability.

### 1.2. Design of Engineered CD3 Epsilon Subunits

FIG. 2B illustrates the structure of an engineered CD3 epsilon (i.e. CD3e) subunit, which substantially comprises a target-recognizing region (e.g., scFv, sdAb, ligand, etc.) fused to the N-terminus of a truncated CD3 epsilon (i.e. “ΔCD3e”, which lacks the intracellular domain corresponding to amino acids 151-179 of human CD3e (SEQ ID NO: 5)). Depending on different embodiments of the engineered CD3e subunit as provided herein, one or more functional regions (e.g., CD40 motif, 4-1BB motif, LAT motif, etc.; as illustrated by the box with dotted lines in FIG. 2B) may further optionally be fused to the C-terminus of the truncated CD3e, but certain embodiment of the engineered CD3e subunit as provided herein may comprise no other functional regions (i.e. the C-terminus of the truncated CD3 epsilon subunit “ΔCD3e” is the C-terminus of the engineered CD3e subunit). Between the target-recognizing region and the truncated CD3e, and/or between the truncated CD3e and the one or more functional regions, a flexible linker (as illustrated by the straight lines in FIG. 2B) can be optionally arranged but not necessary. Any one embodiment of the engineered CD3e subunits as described above may be used alone or in combination with any one of the engineered CD3z subunits as illustrated in FIG. 2A and described above to engineer an immune cell.

### 1.3. Design of Engineered Ligand-Based Aspire-TCR Subunits

FIG. 2C illustrates the structure of a series of embodiments of a ligand-based Aspire-TCR subunit, which substantially comprises a ligand moiety (“ligand”; e.g. IL13 (E13Y), GM-CSF, etc.) fused to the N-terminus of a TCR signaling complex subunit (e.g., one of a TCR alpha subunit, a TCR beta subunit, a CD3 gamma subunit, a CD3 delta subunit, or a CD3 epsilon subunit; illustrated as “TCRα/TCRb/CD3g/CD3d/CD3e” in FIG. 2C). In each of these embodiments, the ligand moiety is substantially the target-recognizing moiety, which can specifically recognize the corresponding cognate receptor expressed on a target cell. A flexible linker, such as a GS linker, may optionally be arranged between the ligand moiety and the TCR signaling complex subunit. It is noted that in addition to fusing to the N-terminus of the TCR complex subunit, the ligand moiety can be inserted at any other locations within the extracellular domain of the TCR signaling complex subunit. Upon expression in an immune cell, the ligand moiety of the engineered target-recognizing TCR complex subunit may direct such engineered immune cell for the specific recognition of a target cell expressing the cognate ligand receptor, in turn allowing the immune cell to exert its cytotoxicity against the target cell. Any one embodiment of the engineered ligand-based Aspire-TCR subunits as described above may be used alone or in combination with an engineered CD3z subunit as illustrated in FIG. 2A to engineer an immune cell.

### Example 2. Ligand-Based IL13Ra2-Targeting Aspire-TCR Subunits, Expression and Characterizations in T Cells

In this example, a ligand-based Aspire-TCR subunit is provided, which substantially encodes a fusion protein “IL13(E13Y)-CD3e”, comprising an “IL13(E13Y)” ligand moiety (SEQ ID NO: 4) fused to the N-terminus of a human CD3 epsilon subunit (SEQ ID NO: 5) by means of a GS linker (SEQ ID NO: 6). The “IL13(E13Y)” ligand moiety is designed to specifically target IL-13Rα2 (or IL13Ra2), a well-established cell surface marker for gliomas and renal cell carcinomas (RCC). To be more specific, the “IL13(E13Y)” ligand moiety comprises a mutant form of IL-13 cytokine, i.e. IL-13 (E13Y), that has been shown to have selective affinity to IL-13Rα2, while less likely to bind to the IL-13Rα1/IL-4β/γc receptor in normal tissues. Moreover, an IL13(E13Y) ligand has been employed in an IL-13Rα2-CAR-T cell therapy for treating glioma, as described in detail in U.S. Pat. No. 7,514,537B, whose disclosure is incorporated herein by reference in its entirety.

### 2.1. Design of Four Aspire-TCR Constructs Targeting IL13Ra2

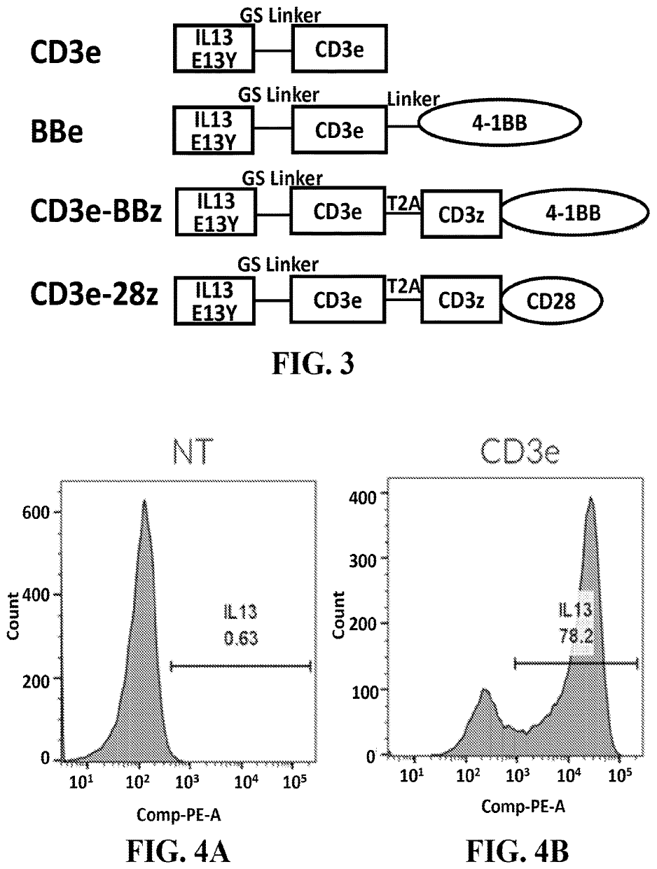

Four Aspire-TCR constructs, including “CD3e”, “BBe”, “CD3e-BBz”, and “CD3e-28z”, as illustrated in FIG. 3, were designed. Description for these four constructs is provided below:
(1) “CD3e” construct: encoding the fusion protein “IL13(E13Y)-CD3e” as described above.
(2) “BBe” construct: encoding a fusion protein “IL13(E13Y)-CD3e-41BB”, where the C-terminus of “IL13(E13Y)-CD3e” is further fused to a 4-1BB intracellular domain (“41BB”; SEQ ID NO: 3) via a linker (“Linker”; SEQ ID NO: 7).
(3) “CD3e-BBz” construct: encoding two fusion proteins “IL13(E13Y)-CD3e” and “CD3z-41BB”, separated by a 2A self-cleaving peptide T2A (SEQ ID NO: 8). The “CD3z-41BB” (i.e. “BBz”) is substantially an embodiment of the engineered CD3z subunit as shown in FIG. 2A, where the C-terminus of the human CD3z subunit (SEQ ID NO: 1) is fused with a human 4-1BB intracellular domain (SEQ ID NO: 3).
(4) “CD3e-28z” construct: encoding two fusion proteins “IL13(E13Y)-CD3e” and “CD3z-CD28”, separated by a 2A self-cleaving peptide T2A (SEQ ID NO: 8). The “CD3z-CD28” (i.e. “28z”) is yet another embodiment of the engineered CD3z subunit as shown in FIG. 2A, where the C-terminus of human CD3z subunit (SEQ ID NO: 1) is fused with a human CD28 co-stimulatory region (SEQ ID NO: 2).

In the “CD3e-BBz” and “CD3e-28z” constructs, the IL13(E13Y)-CD3e and the engineered CD37 subunit can be expressed separately when T2A peptide undergoes self-cleaving.

### 2.2. Expression of the Four IL13(E13Y) Aspire-TCR Constructs in T Cells

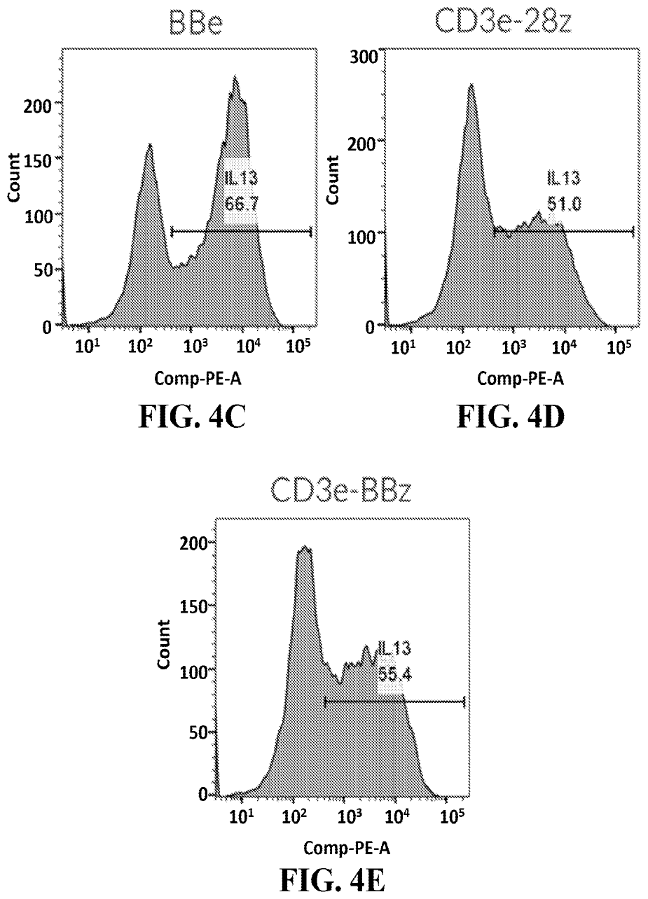

First, in vitro expression of the above four constructs in T cells was examined. Briefly, primary human T cells were transduced with genetic constructs encoding the four Aspire-TCR constructs and expanded in the presence of IL-2 for 12 days. The T cells expressing Aspire-TCR constructs are hereby named “Aspire-T” cells. After expansion, IL13 expression in these IL13(E13Y) Aspire-T cells was measured by flow cytometry, representing the expression of the Aspire-TCR subunit comprising the IL13(E13Y) ligand. As shown in FIGS. 4A-4E, each of the four Aspire-TCR constructs “CD3e”, “BBe”, “CD3e-28z”, and “CD3e-BBz” (FIGS. 4B-4E) can be effectively expressed as compared with the non-transduced (“NT”) T cells (FIG. 4A).

### 2.3. Cytotoxicity and Activation of IL13(E13Y) Aspire-T Cells Against Target Tumor Cells

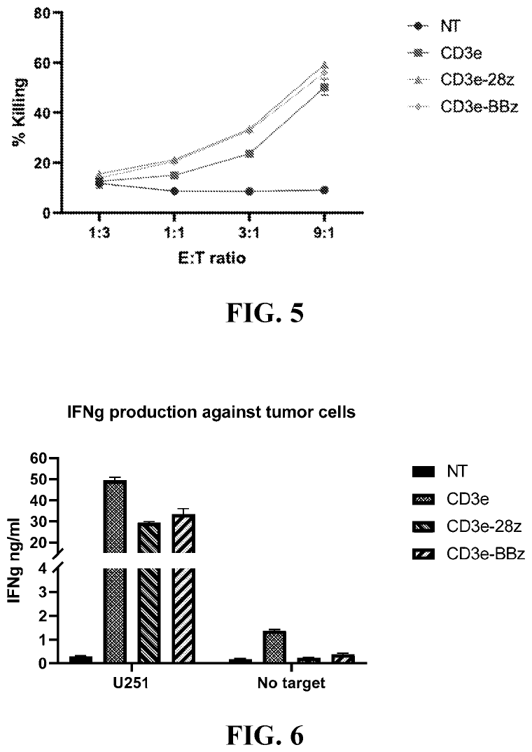

The cytotoxicity of IL13(E13Y) Aspire-T cells against target tumor cells was further investigated. Briefly, different Aspire-T cells were individually cultured overnight with CFSE pre-labeled tumor cells, specifically U251 MG glioma cell line as target tumor cells. Subsequently, tumor cells were collected for viability assessment by 7-AAD. As shown in FIG. 5, three IL13(E13Y) Aspire-T cells, “CD3e”, “CD3e-28z” and “CD3e-BBz”, showed significant cytotoxicity against the IL13Ra2-expressing U251 glioblastoma cells in comparison to the “NT” T cell control. Even more, the “CD3e-28z” and “CD3e-BBz” Aspire-T cells, both featuring the engineered CD3z subunit in addition to the engineered target-recognizing subunit “IL13(E13Y)-CD3e”, demonstrated significantly elevated cytotoxicity against the target tumor cells in comparison to the “CD3e” Aspire-T cells that only expressed the engineered target-recognizing subunit “IL13(E13Y)-CD3e”. This observation indicates that armoring of the T cells expressing the engineered target-recognizing TCR subunit with the engineered CD3z subunit can enhance cytotoxicity against the target tumor cells.

To assess the antigen-specific stimulation of the various Aspire-T cells, the following assay was conducted. Briefly, different Aspire-T cells were cultured with or without U251 target tumor cells for 48 hours. Subsequently, the supernatant was collected and IFN-γ levels were measured by ELISA. As shown in FIG. 6, in the presence of target tumor cells (i.e. the “U251” group), the “NT” T cells were not activated by the antigen, whereas the three IL13(E13Y) Aspire-T cell types, “CD3e”, “CD3e-28z”, and “CD3e-BBz”, all exhibited a notable increase in IFN-γ levels, indicating a significant antigen activation. However, it is noteworthy that in the absence of the target tumor cells (i.e. the “No target” group), the “CD3e” T cells (i.e. T cells expressing the engineered target-recognizing subunit “IL13(E13Y)-CD3e” only) showed a relatively high background activation (i.e. activation in the absence of antigen stimulation), which is approximately 3-10 folds higher compared with other groups. Specifically, the “CD3e-28z” T cells (i.e. T cells co-expressing the engineered target-recognizing subunit IL13(E13Y)-CD3e and the engineered “CD3z-CD28” subunit) showed almost no activation without antigen stimulation (i.e. a level comparable to the control “NT” T cells), whereas the “CD3e-BBz” T cells (i.e. T cells co-expressing the engineered target-recognizing subunit IL13(E13Y)-CD3e and the engineered “CD3z-4-1BB” subunit) showed a little higher background activation than the “CD3e-28z” T cells, but it was still much less than the “CD3e” T cells (i.e. only approximately ⅕ of the “CD3e” T cells). These results indicate that armoring of the T cells expressing the engineered target-recognizing TCR subunit with the engineered CD3z subunit can reduce the unwanted background activation.

### 2.4. Cytotoxicity and Activation of IL13(E13Y) Aspire-T Cells Against Non-Target Tumor Cells

To further examine the specificity of the four Aspire-T cells, their cytotoxicity and activation against non-target cells were also investigated.

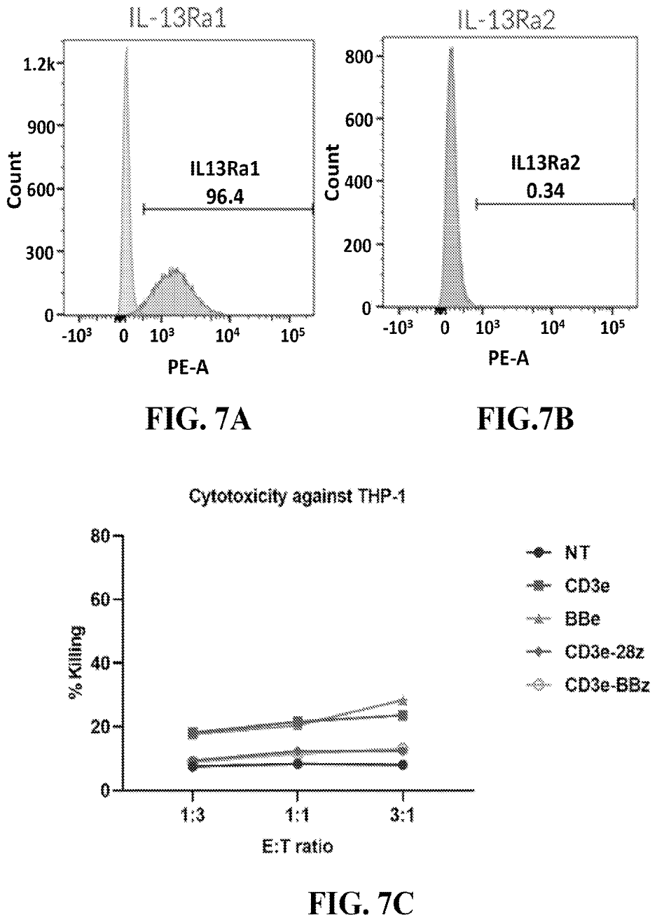

First, the T cell cytotoxicity against non-target cells was examined, and a non-target tumor cell line THP-1, which expresses IL13Ra1 instead of IL13Ra2 (see FIGS. 7A-7B), was tested. As shown by the results in FIG. 7C, both the “CD3e” (i.e. T cells expressing the engineered target-recognizing subunit “IL13(E13Y)-CD3e” only) and “BBe” (i.e. T cells expressing the engineered target-recognizing subunit “IL13(E13Y)-CD3e-4-1BB” subunit) Aspire-T cells showed a relatively higher cytotoxicity against the non-target THP-1 cells, whereas the “CD3e-28z” (i.e. T cells co-expressing the engineered target-recognizing subunit “IL13(E13Y)-CD3e” and the engineered “CD3z-CD28” subunit) and the “CD3e-BBz” (i.e. T cells co-expressing the engineered target-recognizing subunit “IL13(E13Y)-CD3e” and the engineered “CD3z-4-1BB”) Aspire-T cells showed much lower cytotoxicity, which was substantially similar to the cytotoxicity of “NT” control T cells.

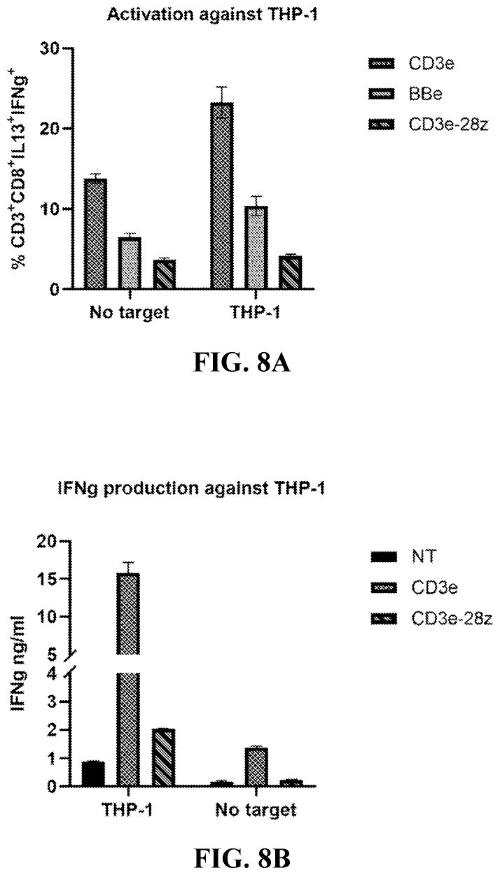

Moreover, the T cell activation against the non-target THP-1 cells was further investigated. As shown by the results in FIG. 8A, both “CD3e” and “BBe” Aspire-T cells showed elevated activation upon contact with the non-target THP-1 cells, as illustrated by the percentage increase of the IFN-γ-producing cells in the presence of THP-1 cells (i.e. “THP-1 group) as compared to that in the absence of THP-1 cells (i.e. “No target” group). In sharp contrast, the “CD3e-28z” Aspire-T cells showed no activation. In an independent experiment shown in FIG. 8B, which examined the IFN-γ secretion levels, the “CD3e” Aspire-T cells showed obvious activation upon contacting with the non-target THP-1 cells, whereas the “CD3e-28z” Aspire-T cells showed only marginal activation.

The above results in FIGS. 7A-7C and FIGS. 8A-8B suggest that armoring of the T cells expressing the engineered target-recognizing TCR subunit with the engineered CD3z subunit can significantly reduce the unwanted activation by, and cytotoxicity against, non-target cells.

Taken the above together, the Aspire-T cells expressing only an engineered CD3e that is fused with the IL13(E13Y) ligand moiety (e.g., “CD3e” and “BBe”) demonstrated cytotoxicity against IL13Ra2-expressing tumor cells (target tumor cells) (FIG. 5). However, these Aspire-T cells exhibited a certain level of background activation even in the absence of antigen or can even be activated in the presence of, and thereby causing cytotoxicity against, non-target cells (FIG. 7C). The limited level of specificity of these Aspire-T cells can nonetheless be rescued by co-expression of the engineered CD3z subunit provided herein (i.e. CD3z fused with CD28 or 4-1BB co-stimulatory domain), which can dramatically reduce the background activation of the engineered T cells in the absence of antigen (see FIG. 6), significantly reduce unwanted cytotoxicity against non-target cells (see FIGS. 7C, 8A and 8B), and furthermore increase the cytotoxicity against target tumor cells (see FIG. 5), thereby providing a favorably safer therapeutic profile.

### Example 3. ScFv-Based ALPP-Targeting Aspire-TCR Subunits, Expression and Characterizations in T Cells

In this example, an scFv-based Aspire-TCR subunit is provided, which substantially includes a fusion protein “F8-CD3e”, comprising an anti-ALPP scFv “F8” (SEQ ID NO: 9) fused to the N-terminus of a human CD3 epsilon subunit (SEQ ID NO: 5) via a GS linker (SEQ ID NO: 6). This Aspire-TCR is specifically designed to target ALPP (alkaline phosphatase, placental; also known as placental alkaline phosphatase or PLAP), which has been established as a specific tumor marker for ovarian cancer (e.g., ovarian adenocarcinoma, serous cystadenocarcinoma, undifferentiated carcinoma and dysgerminoma) and seminoma.

### 3.1. Designs of Two Aspire-TCR Constructs Targeting ALPP

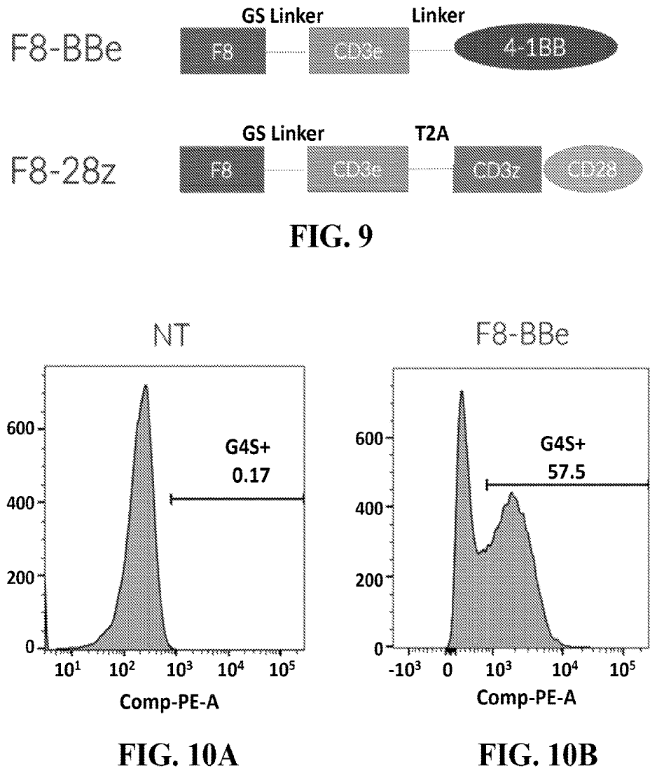

Specifically, two constructs were designed, and their structural diagrams are illustrated in FIG. 9. The two constructs can be separately cloned into a pMP71 retroviral vector to produce engineered T cells. More specifically, these two constructs include:
(1) a “F8-BBe” construct, which substantially encodes a fusion protein “F8-CD3e-4-1BB”, where the C-terminus of the fusion protein “F8-CD3e” as described above is further fused to a 4-1BB intracellular domain (“4-1BB”; SEQ ID NO: 3) via a linker (“Linker”; SEQ ID NO: 7);
(3) a “F8-28z” construct, which substantially encodes two fusion proteins “F8-CD3e” and “CD3z-CD28”, separated by a 2A self-cleaving peptide T2A (SEQ ID NO: 8). Herein, the “CD3z-CD28” is the same as the one described above in Example 2, where the C-terminus of human CD3z subunit (SEQ ID NO: 1) is fused with a human CD28 co-stimulatory region (SEQ ID NO: 2).

### 3.2. Expression of Anti-ALPP Aspire-TCR Constructs in T Cells

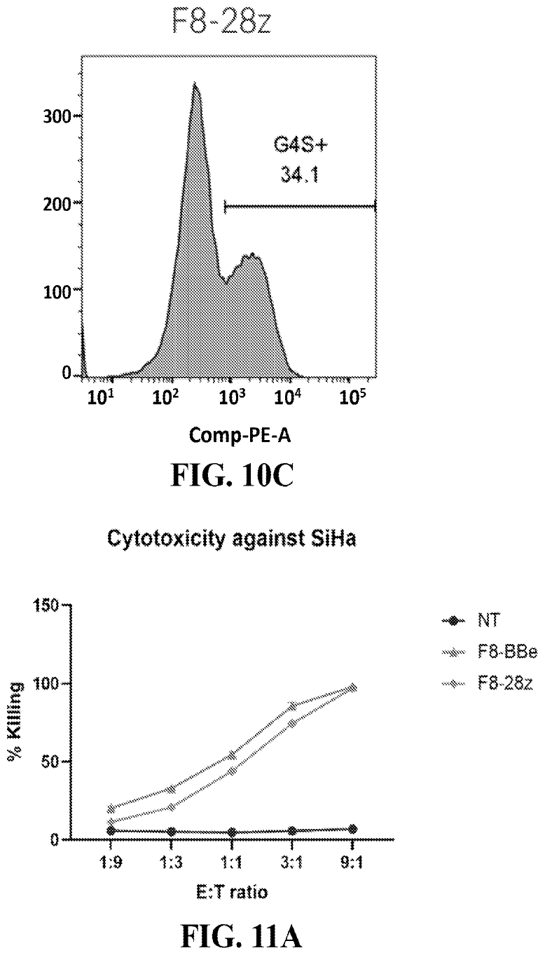

In vitro expression of these two Aspire-TCR constructs in T cells was examined, using non-transduced (“NT”) T cells as a negative control. Briefly, primary human T cells were transduced with different constructs and expanded in the presence of IL-2 for 5-6 days. After expansion, F8 scFv expression was measured by flow cytometry. As shown in FIGS. 10A-10C, both Aspire-TCR constructs can be effectively expressed in transduced T cells.

### 3.3. Cytotoxicity and Activation of Anti-ALPP Aspire-T Cells Against Target Tumor Cells

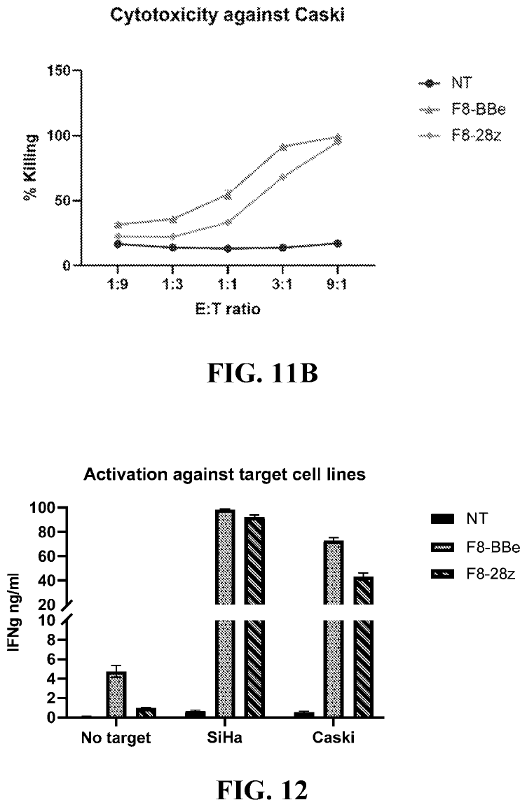

The cytotoxicity of anti-ALPP Aspire-T cells against target tumor cells was further investigated. Briefly, different Aspire-T cells were respectively cultured overnight with CFSE pre-labeled tumor cells, specifically SiHa or Caski as target tumor cells. Subsequently, tumor cells were collected for viability assessment by 7-AAD. As shown in FIGS. 11A-11B, compared with the “NT” control T cells, both “F8-28z” and “F8-BBe” Aspire-T cells showed significant cytotoxicity against both SiHa cells (FIG. 11A) and Caski cells (FIG. 11B), respectively.

To examine the antigen-specific stimulation of the various Aspire-T cells, Aspire-T cells were respectively cultured with or without target tumor cells (SiHa or Caski) for 48 hours. Subsequently, the supernatant was collected and the IFN-γ levels were measured by ELISA. The results are shown in FIG. 12. As expected, the control “NT” T cells showed no activation either in the “No target” group or in any of the target tumor groups (SiHa and Caski tumor cells). Both “F8-28z” and “F8-BBe” Aspire-T cells showed antigen-specific activation evidenced by the increased IFN-γ secretion in the presence of target tumor cells (with antigens) as compared to that in the absence of antigens (the “No target” group). The “F8-BBe” Aspire-T cells (expressing only the target-recognizing “F8-CD3e-4-1BB” subunit) showed a relatively higher level of background activation in the absence of antigen, whereas the “F8-28z” Aspire-T cells (co-expressing the target-recognizing “F8-CD3e” subunit and the engineered “CD3z-CD28” subunit) only showed minimal background activation in the absence of antigens. These results indicate that armoring of the T cells expressing the engineered target-recognizing TCR subunit with the engineered CD3z subunit can reduce unwanted background activation. While both “F8-28z” and “F8-BBe” Aspire-T cells showed good antigen-specific activation, the “F8-28z” T cells showed favorably minimal background activation in the absence of antigen stimulation.

### 3.4. Cytotoxicity and Activation of Anti-ALPP Aspire-T Cells Against Non-Target Tumor Cells

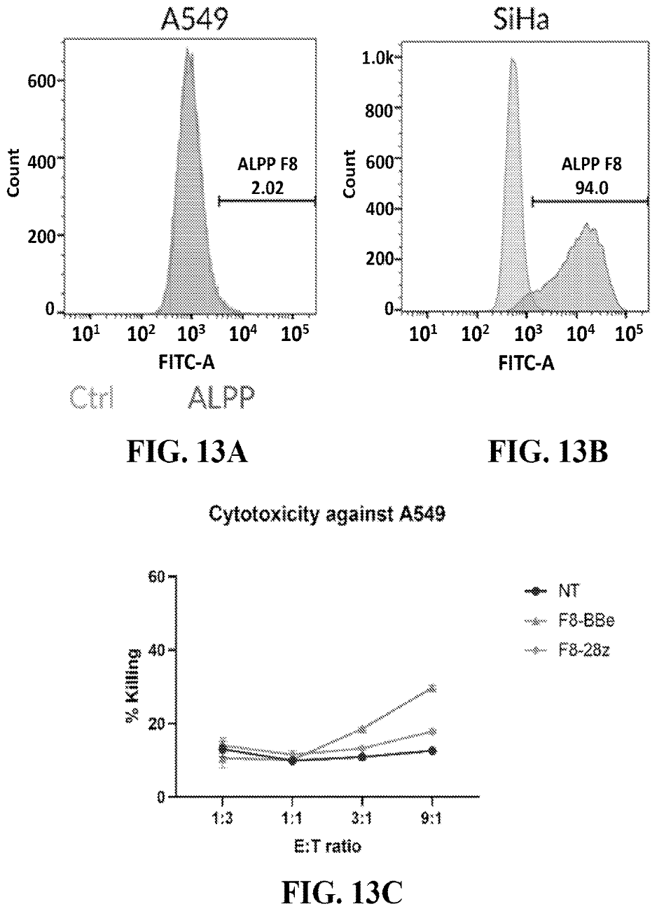

To further examine the specificity of the two anti-ALPP Aspire-T cell types, their cytotoxicity against non-target cells were further investigated. As shown in FIGS. 13A-13B, as compared to the target SiHa cells tested positive for the antigen ALPP (FIG. 13B), A549 cells were non-target tumor cells tested negative for the antigen ALPP (FIG. 13A). Therefore, A549 cells were used to assess non-specific cytotoxicity of the two anti-ALPP Aspire-T cells. As shown by the results in FIG. 13C, the “F8-BBe” T cells (expressing only the target-recognizing “F8-CD3e-4-1BB” subunit) showed relatively higher cytotoxicity against the non-target A549 cells. In contrast, the “F8-28z” T cells (co-expressing the target-recognizing “F8-CD3e” subunit and the engineered “CD3z-CD28” subunit) showed much lower off-target cytotoxicity, which was substantially similar to the cytotoxicity of the control “NT” T cells.

Therefore, similar to Example 2 described above, although the anti-ALPP Aspire-T cell “F8-BBe” (expressing only the target-recognizing “F8-CD3e-4-1BB” subunit) exhibited cytotoxicity against SiHa or Caski target tumor cells (FIGS. 11A-11B), they also showed a relatively high level of background activation in the absence of antigen (FIG. 12) and a relatively high level of off-target cytotoxicity against the non-target tumor cells (FIG. 13C). This limited specificity of “F8-BBe” Aspire-T cells can be significantly improved or even rescued by co-expressing the engineered “CD3z-CD28” subunit. This co-expression of both the anti-ALPP TCR subunit and the engineered CD3z-CD28 subunit in the “F8-28z” Aspire-T cell dramatically reduced the background activation in the absence of antigen (FIG. 12) and diminished the off-target cytotoxicity against non-target cells (FIG. 13C), thereby providing a favorably safer therapeutic profile.

### Example 4. Truncated CD3e-Based Aspire-TCR Subunits, Expression and Characterizations in T Cells

This example substantially provides a series of truncated CD3e-based engineered CD3 epsilon (i.e. CD3e) subunits according to different embodiments of the engineered CD3e subunit as described above in Example 1 and illustrated in FIG. 2B. These engineered CD3e subunits all comprise a target-recognizing region fused to the N-terminus of a truncated CD3 epsilon (“ΔCD3e” (SEQ ID NO: 10), which lacks the C-terminal 29 amino acid residues compared with full-length CD3e subunit (SEQ ID NO: 5)), thereby forming a fusion polypeptide “target-recognizing region-ΔCD3e”. Depending on different embodiments, one or more functional regions (e.g., CD40 motif, 4-1BB motif, LAT motif, etc.) can further optionally be fused to the C-terminus the fusion polypeptide “target-recognizing region-ΔCD3e”.

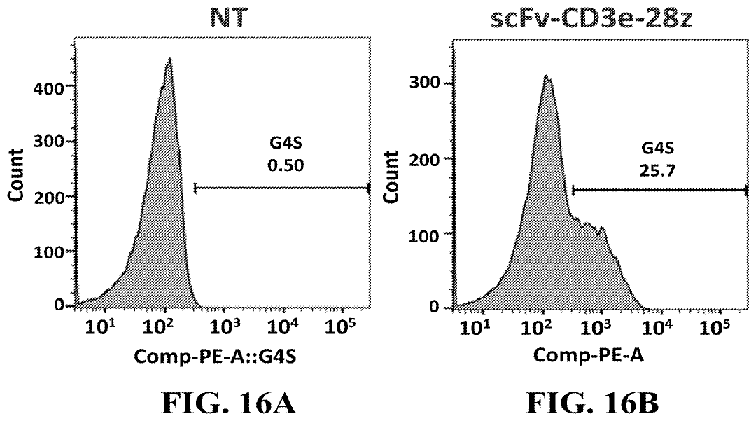

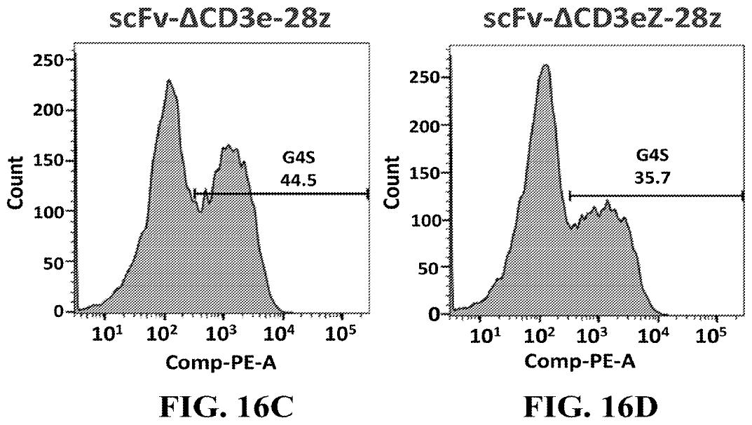

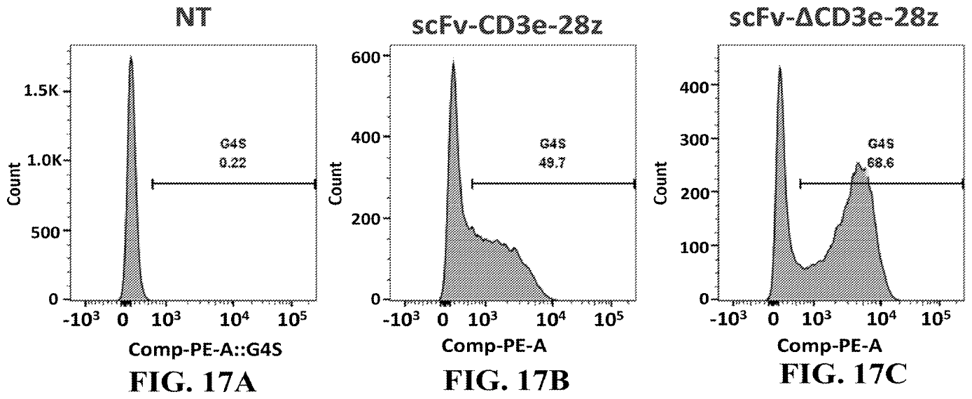

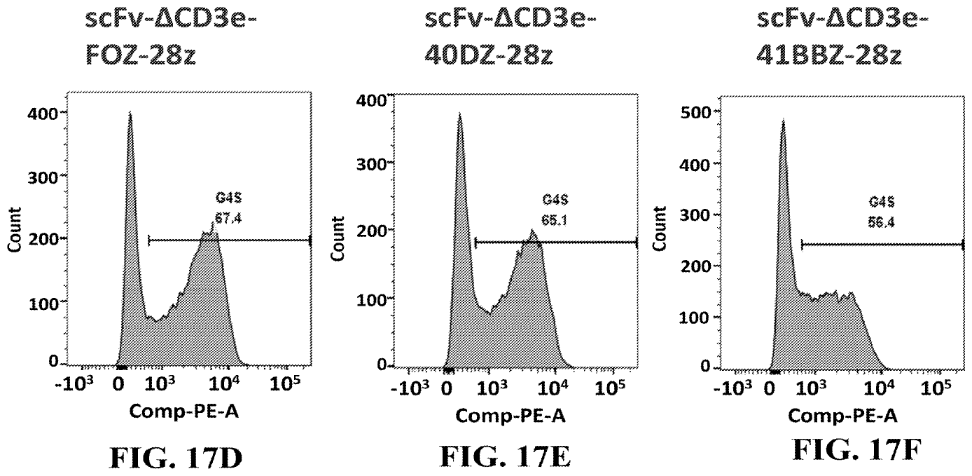

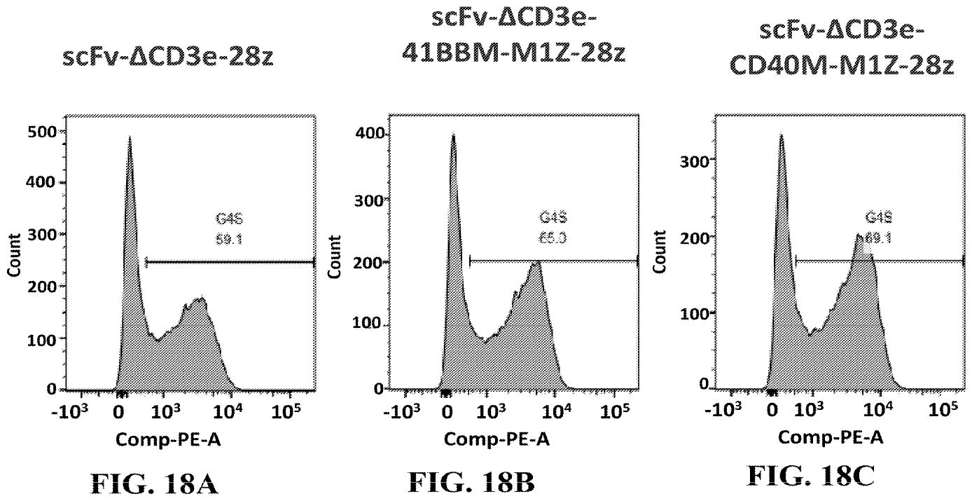

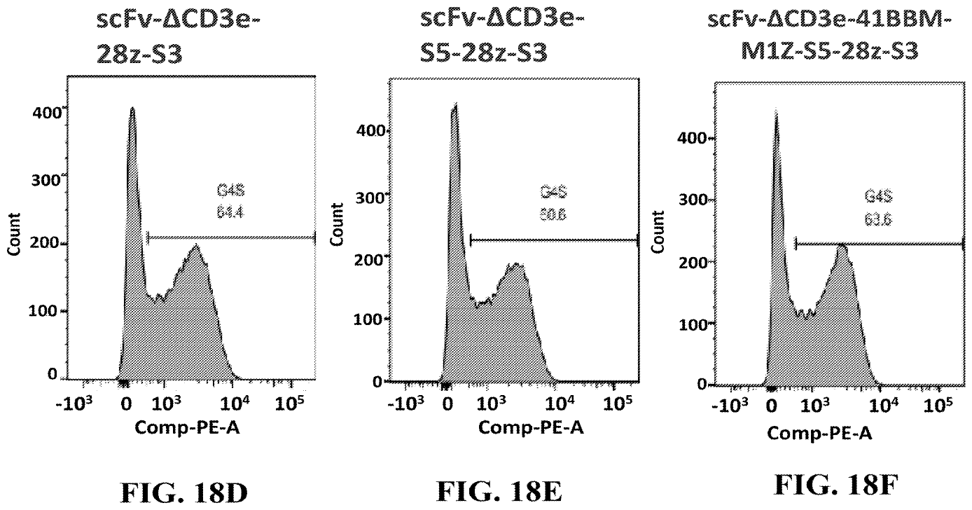

In this example, an anti-ALPP scFv was used as the target-recognizing region for the engineered CD3e subunits, which may be an F8 scFv (SEQ ID NO: 9) or an A02 scFv (SEQ ID NO: 30, disclosed in US20220125845A1, whose contents are incorporated by reference in entirety). The F8 scFv was used for all the in vitro experiments in FIGS. 16A-23B, and the A02 scFv is used for the in vivo experiments in FIGS. 25-27.

### 4.1. Designs of Aspire-TCR Constructs

As shown in FIG. 14 and Table 1, a total of 11 Aspire-TCR constructs A-K were designed, each encoding two chimeric proteins including an engineered CD3e subunit and an engineered CD3z subunit, that are separated by a T2A linker (SEQ ID NO: 8). Each of these constructs can be cloned into a pMP71 retroviral vector to thereby produce the Aspire-T cells.

Herein, the engineered CD3e subunit in each construct is substantially a target-recognizing Aspire-TCR subunit, comprising an scFv fused, via a flexible linker (e.g., a GS linker (SEQ ID NO: 6)), to a full-length CD3e subunit (see Design A) or to a truncated CD3e (“ΔCD3e”) subunit (see Designs B-K, with various domains/motifs/regions in each different embodiment). The engineered “CD3z-CD28” subunit, or “28z”, comprises a CD3z or modified CD3z subunit with the intracellular domain further comprising a CD28 co-stimulatory domain “CD28”. In the examples below, these two engineered CD3 subunits were co-expressed in T cells and were characterized in vitro and in vivo. It is noted that Design A is substantially the same as the “F8-CD3e” construct in Example 3 above, which is mainly used as control to characterize Designs B-K in this example.

The following domains/motifs/regions may be included in the designs: (1) “scFv”; (2) human CD3e component: “CD3e” (SEQ ID NO: 5), or “ΔCD3e” (i.e. CD3e with an intracellular region thereof truncated; SEQ ID NO: 10); (3) human CD3z signaling domain: “CD3z ITAM2-3” (SEQ ID NO: 12), “CD3z ITAM3” (SEQ ID NO: 11), “CD3z-LHYRHQMQ” (SEQ ID NO: 20), “CD3z ITAM2-3-LHYLSLMQ” (SEQ ID NO: 18), or “CD3z ITAM3-LHYLSLMQ” (SEQ ID NO: 19); (4) human 4-1BB functional region(s): “4-1BB motif” (SEQ ID NO: 15) or “4-1BB” (short as “4-1BB intracellular domain”; SEQ ID NO: 3); or CD28 co-stimulatory domain (“CD28”; SEQ ID NO: 2); and (5) different modified regions, domains or motifs intended to enhance Aspire-T cell functions: “FcεR1γ” (i.e. FcεR1γ intracellular domain; SEQ ID NO: 25); “OX40” (i.e. OX40 intracellular domain; SEQ ID NO: 26); “FceR1g+OX40” (i.e. a compound functional region formed by fusing “FcεR1γ” with “OX40” from N- to C-terminus; SEQ ID NO: 13), “CD40” (i.e. CD40 intracellular domain; SEQ ID NO: 27); “DAP2” (i.e. DAP2 intracellular domain; SEQ ID NO: 28); “CD40+DAP12” (i.e. a compound functional region formed by fusing “CD40” with “DAP2” from N- to C-terminus; SEQ ID NO: 14), “CD40 motif” (SEQ ID NO: 17), and “LAT motif” (SEQ ID NO: 16). The below table summarize the structures of these 11 Aspire-TCR constructs (Designs A-K).

TABLE 1
Structures of 11 Aspire-TCR constructs (A-K)

| | | | engineered |
|---|---|---|---|
| | | engineered | CD3z |
| Design | Construct | CD3e subunit | subunit |
| [A] | scFv-CD3e-28z | scFv-CD3e | 28z |
| [B] | scFv-ΔCD3e-28z | scFv-ΔCD3e | 28z |
| [C] | scFv-ΔCD3eZ-28z | scFv-ΔCD3eZ | 28z |
| [D] | scFv-ΔCD3e-FOZ-28z | scFv-ΔCD3e-FOZ | 28z |
| [E] | scFv-ΔCD3e-40DZ-28z | scFv-ΔCD3e-40DZ | 28z |
| [F] | scFv-ΔCD3e-41BBMZ- | scFv-ΔCD3e- | 28z |
| | 28z | 41BBMZ | |
| [G] | scFv-ΔCD3e-41BBM- | scFv-ΔCD3e-41BBM- | 28z |
| | M1Z-28z | M1Z | |
| [H] | scFv-ΔCD3e-CD40M- | scFv-ΔCD3e-CD40M- | 28z |
| | M1Z-28z | M1Z | |
| [I] | scFv-ΔCD3e-28z-S3 | scFv-ΔCD3e | 28z-S3 |
| [J] | scFv-ΔCD3e-S5-28z-S3 | scFv-ΔCD3e-S5 | 28z-S3 |
| [K] | scFv-ΔCD3e-41BBM- | scFv-ΔCD3e-41BBM- | 28z-S3 |
| | M1Z-S5-28z-S3 | M1Z-S5 | |

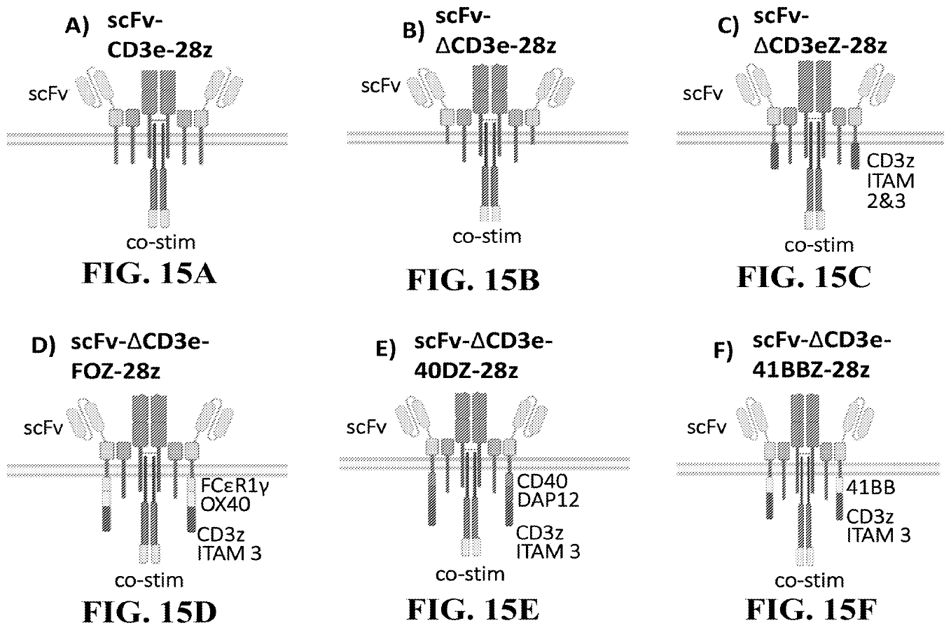

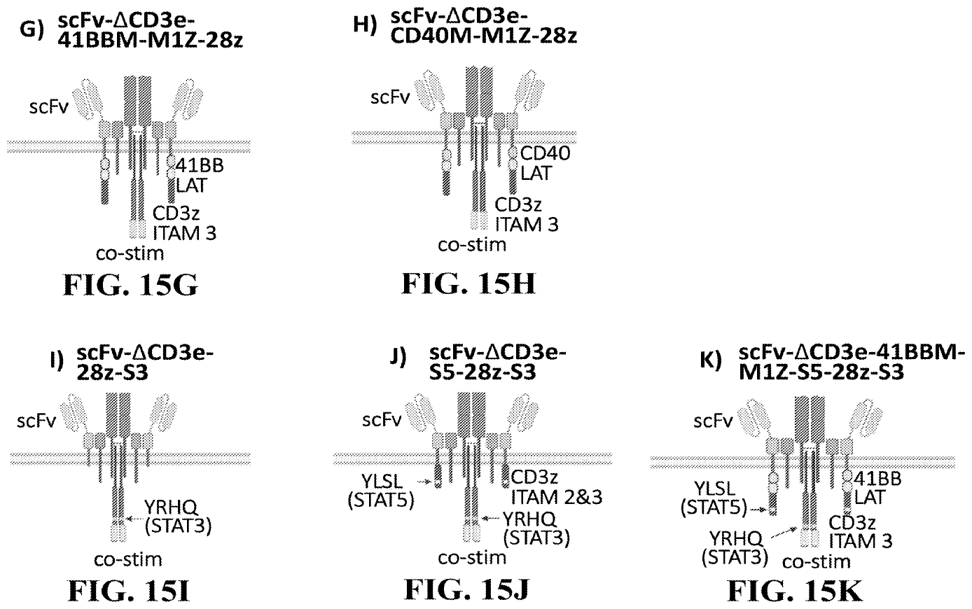

As shown in FIGS. 15A-15K, the engineered target-recognizing CD3e subunit and the engineered CD3z subunit encoded by each of the 11 Aspire-TCR constructs shown in FIG. 14 can, when expressed in an immune cell, be incorporated into a T cell receptor signaling complex, thereby forming an Aspire-TCR complex.

### 4.2. In Vitro Expression of Aspire-TCR Constructs in T Cells

In vitro expression of these constructs in T cells was examined. Briefly, primary human T cells were transduced with different constructs and expanded in the presence of IL-2 for 12-13 days. After expansion, scFv expression on different Aspire-T cells was measured by flow cytometry. As shown in FIGS. 16A-16D, 17A-17F, and 18A-18F, compared to scFv-CD3e-28z (Design A in FIG. 14), all the Aspire-TCRs comprising “ΔCD3e” (Designs B-K) showed unexpectedly better expression in primary T cells, indicating that the C-terminal 29-amino acid (AA) portion of CD3e might negatively impact the expression level of the engineered CD3e subunit, and that removal of this 29-AA portion from the CD3e component can significantly enhance expression of the engineered CD3e subunit.

### 4.3. In Vitro Expansion Capability and Post-Expansion Memory Phenotyping of Aspire-T Cells

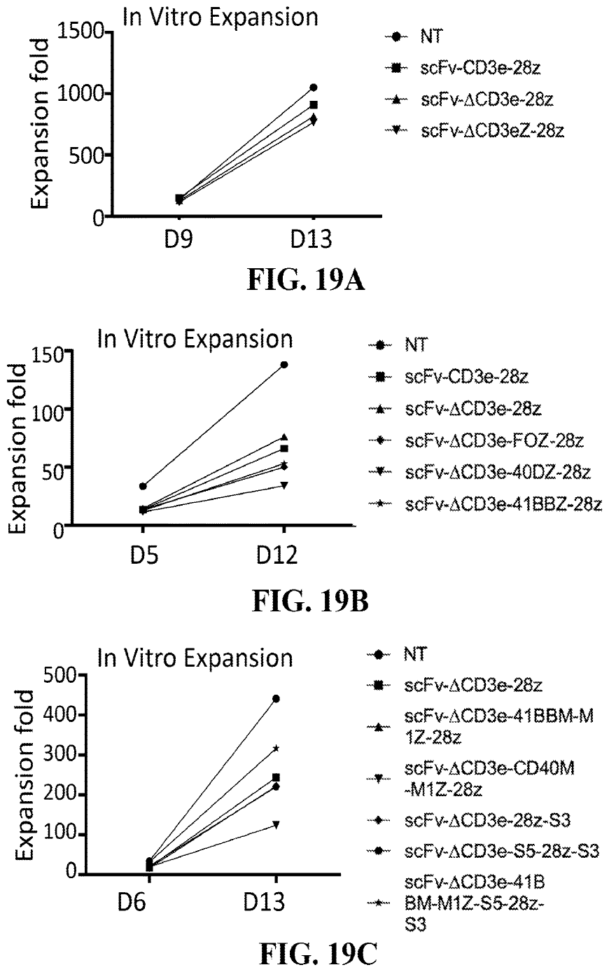

The in vitro expansion capability of scFv Aspire-T cells was investigated, as described below. Primary human T cells were transduced with individual scFv Aspire-TCR constructs designed in FIG. 14 and expanded in the presence of IL-2 for 12-13 days. Expansion folds were calculated when cells were harvested. As shown in FIGS. 19A-19C, all Aspire-T cells with “ΔCD3e” (Designs B-K) expanded well in culture, however, the expansion fold varied. In general, the control non-transduced (“NT”) T cells displayed the best expansion capability compared to all transduced Aspire-T cells. Compared to T cells expressing scFv-CD3e-28z (Design A), scFv-ΔCD3e-28z (Design B) and scFv-ΔCD3eZ-28z (Design C) showed similar T cell expansion levels (FIG. 19A). When compared to T cells expressing scFv-ΔCD3e-28z (Design B), T cells expressing scFv-ΔCD3e-FOZ-28z (design D) or scFv-ΔCD3e-41BBZ-287. (Design F) showed slightly lower expansion levels, and T cells expressing scFv-ΔCD3e-40DZ-28z (Design E) showed much lower expansion levels (FIG. 19B). Moreover, when compared to T cells expressing scFv-ΔCD3e-28z (Design B), T cells expressing scFv-ΔCD3e-41BBM-M1Z-28z (Design G), scFv-ΔCD3e-28z-S3 (Design I) or scFv-ΔCD3e-S5-28z-S3 (Design J) showed a similar level of expansion capability, while T cells expressing scFv-ΔCD3e-CD40M-M1Z-28z (Design H) expanded slower and T cells expressing scFv-ΔCD3e-41BBM-M1Z-S5-28z-S3 (Design K) expanded faster in culture (FIG. 19C).

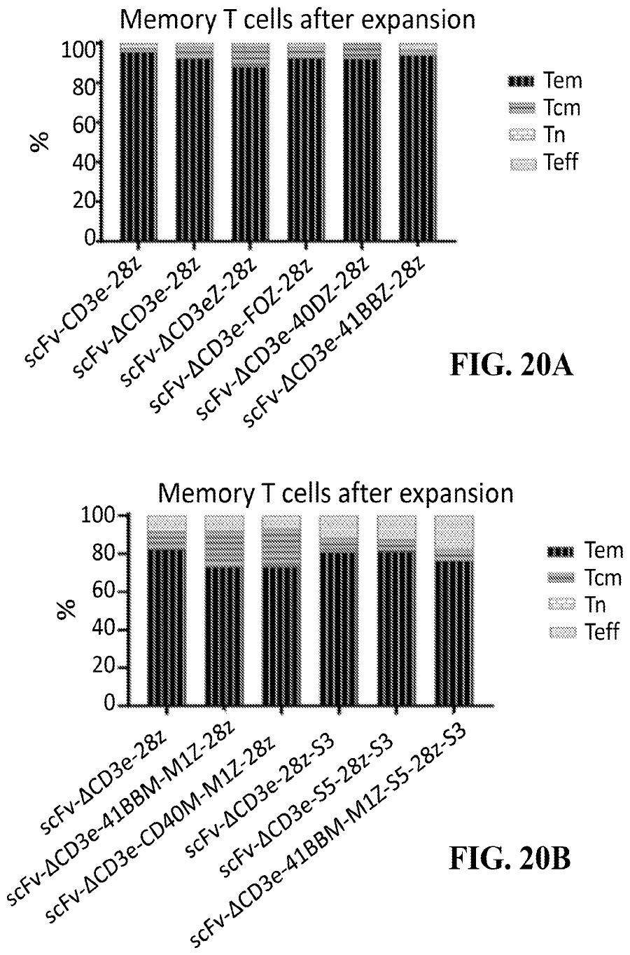

Furthermore, the proportion of memory T cells in different Aspire-T cells after expansion was investigated as follows. Briefly, expanded Aspire-T cells were stained with CD3, CD8, G4S, CD45RO, CCR7 antibodies and subsequently analyzed by flow cytometry. Different phenotypes of T cells included effector memory T cells (“Tem”), central memory T cells (“Tcm”), naïve T cells (“Tn”), and terminally differentiated effector T cells (“Teff”). As shown in FIG. 20A, T cells expressing scFv-ΔCD3e-28z (Design B) showed more, i.e. a higher proportion of, “Tcm” (i.e. central memory T cells) than T cells expressing scFv-CD3e-28z (Design A) in culture, indicating that that T cells expressing scFv-ΔCD3e-28z (Design B) retained more Tcm after expansion compared to T cells expressing scFv-CD3e-28z (Design A). When compared to T cells expressing scFv-ΔCD3e-28z (Design B), T cells expressing scFv-ΔCD3e-FOZ-28z (Design D) or scFv-ΔCD3e-40DZ-28z (Design E) showed similar memory phenotyping, while T cells expressing scFv-ΔCD3eZ-28z (Design C) showed a higher proportion of Tcm, and T cells expressing scFv-ΔCD3e-41BBZ-28z (Design F) had slightly less, i.e. a lower proportion of, Tcm but more Teff (i.e. effector T cells). Moreover, as shown in FIG. 20B, T cells expressing scFv-ΔCD3e-41BBM-M1Z-28z (Design G) or scFv-ΔCD3e-CD40M-M1Z-28z (Design H) showed more Tcm in culture than T cells expressing scFv-ΔCD3e-28z (Design B). However, T cells expressing scFv-ΔCD3e-28z-S3 (Design I), scFv-ΔCD3e-S5-28z-S3 (Design J), and scFv-ΔCD3e-41BBM-M1Z-S5-28z-S3 (Design K) showed slightly less Tcm but more Teff. In particular, T cells expressing scFv-ΔCD3e-41BBM-M1Z-S5-28z-S3 (Design K) showed the highest Teff. T cells expressing scFv-ΔCD3e-CD40M-M1Z-28z (Design H) showed higher Tn (naïve T cells) than scFv-ΔCD3e-28z (Design B) or scFv-ΔCD3e-41BBM-M1Z-28z (Design G).

### 4.4. In Vitro Cytotoxicity and Activation of Aspire-T Cells Against Target Tumor Cells

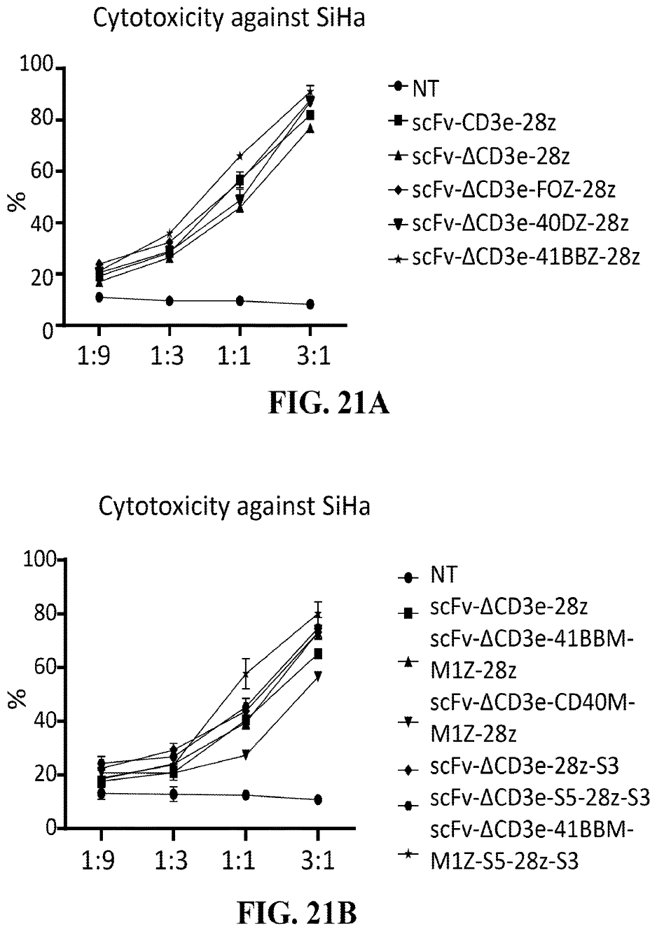

The specific in vitro cytotoxicity against target tumor cells was further examined. Briefly, different Aspire-T cells were respectively cultured with CFSE pre-labeled target tumor cells (SiHa cells) overnight, after which tumor cells were collected, and viability was measured by 7-AAD. As shown in FIG. 21A, no cytotoxicity was detected for the control “NT” T cells, and when compared to T cells expressing scFv-CD3e-28z (Design A), T cells expression scFv-ΔCD3e-28z (Design B), scFv-ΔCD3e-FOZ-28z (Design D), scFv-ΔCD3e-40DZ-28z (Design E), or scFv-ΔCD3e-41BBZ-28z (Design F) showed similar T cell cytotoxicity against target SiHa tumor cells. As shown in FIG. 21B, when compared to T cells expressing scFv-ΔCD3e-28z (Design B), T cells expressing scFv-ΔCD3e-41BBM-M1Z-28z (Design G), scFv-ΔCD3e-28z-S3 (Design I), or scFv-ΔCD3e-S5-28z-S3 (Design J) showed similar T cell killing activity, whereas T cells expressing scFv-ΔCD3e-CD40M-M1Z-28z (Design H) showed lower and T cells expression scFv-ΔCD3e-41BBM-M1Z-S5-28z-S3 (Design K) showed higher T cell killing activity, against tumor target cells.

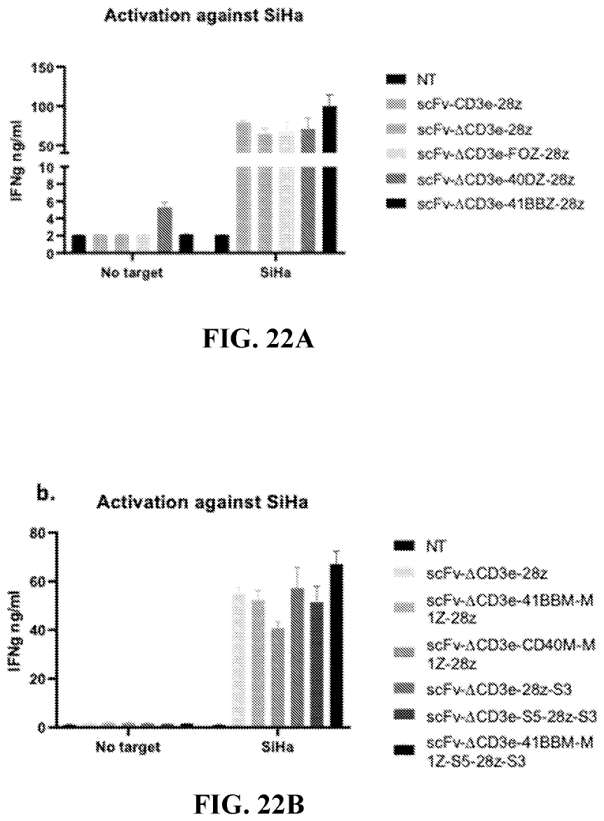

Furthermore, the activation of Aspire-T cells upon antigen-specific stimulation was also examined in vitro. Briefly, different Aspire-T cells were respectively cultured with or without target SiHa tumor cells for 48 hours, after which the supernatant was collected and IFN-γ level was measured by ELISA. As shown in FIG. 22A, when compared to T cells expressing scFv-CD3e-28z (Design A), T cells expressing scFv-ΔCD3e-28z (Design B), scFv-ΔCD3e-FOZ-28z (Design D), scFv-ΔCD3e-40DZ-28z (Design E), or scFv-ΔCD3e-41BBZ-28z (Design F) showed similar T cell activation upon antigen stimulation. However, T cells expressing scFv-ΔCD3e-40DZ-28z (Design E) showed higher background activation. T cells expressing scFv-ΔCD3e-41BBZ-28z (Design F) exhibited higher activation as compared to T cells expressing scFv-ΔCD3e-28z (Design B). As shown in FIG. 22B, when compared to T cells expressing scFv-ΔCD3e-28z (Design B), T cells expressing scFv-ΔCD3e-41BBM-M1Z-28z (Design G), scFv-ΔCD3e-28z-S3 (Design I) and scFv-ΔCD3e-S5-28z-S3 (Design J) showed similar levels of T cell activation, whereas T cells expressing scFv-ΔCD3e-CD40M-M1Z-28z (Design H) showed lower, and T cells expressing scFv-ΔCD3e-41BBM-M1Z-S5-28z-S3 (Design K) showed higher levels, of T cell activation.

### 4.5. Aspire-T Cells Expansion Upon Antigen Repetitive Stimulation In Vitro

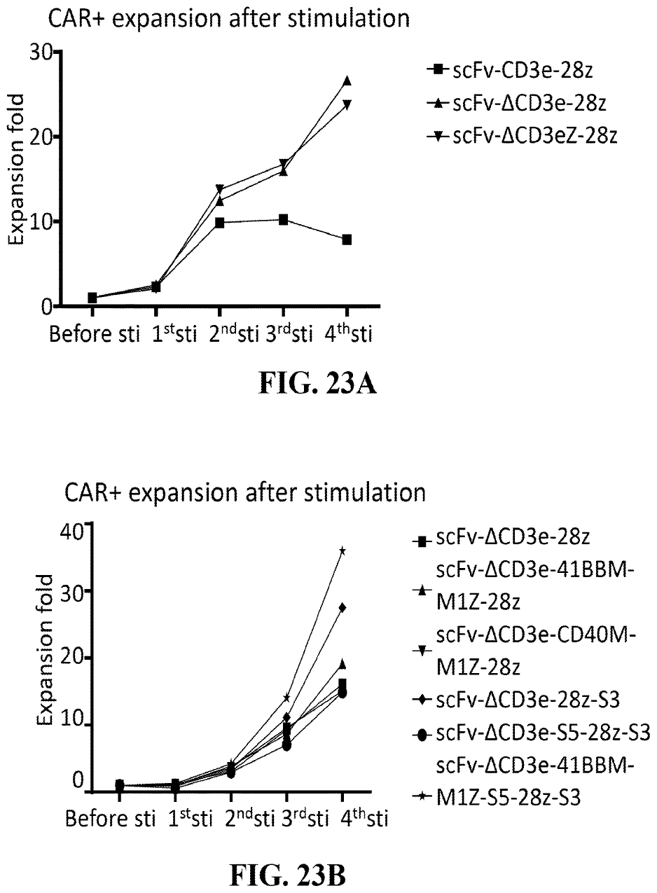

Briefly, different Aspire-T cells were repeatedly stimulated by tumor target cells, and the expansion of CAR+ T cells (i.e. T cells expressing the Aspire-TCR) was monitored. As shown in FIG. 23A, when compared to T cells expressing scFv-CD3e-28z (Design A), T cells expressing scFv-ΔCD3e-287. (Design B) or scFv-ΔCD3eZ-287. (Design C) showed better T cell expansion upon repeated antigen stimulation. As shown in FIG. 23B, T cells expressing scFv-ΔCD3e-41BBM-M1Z-28z (Design G), scFv-ΔCD3e-CD40M-M1Z-28z (Design H) and scFv-ΔCD3e-S5-28z-S3 (Design J) showed similar T cell expansion as compared to T cells expressing scFv-ΔCD3e-28z (Design B) after antigen repetitive stimulation, whereas T cells expressing scFv-ΔCD3e-28z-S3 (Design I) and scFv-ΔCD3e-41BBM-M1Z-S5-28z-S3 (Design K) both showed notably enhanced T cell expansion in comparison to T cells expressing scFv-ΔCD3e-28z (Design B) upon antigen repetitive stimulation.

4.6. In Vivo Antitumor Efficacy and Proliferation Capability of Aspire-T Cells Armored with Membrane-Tethered IL12 (mtIL12)

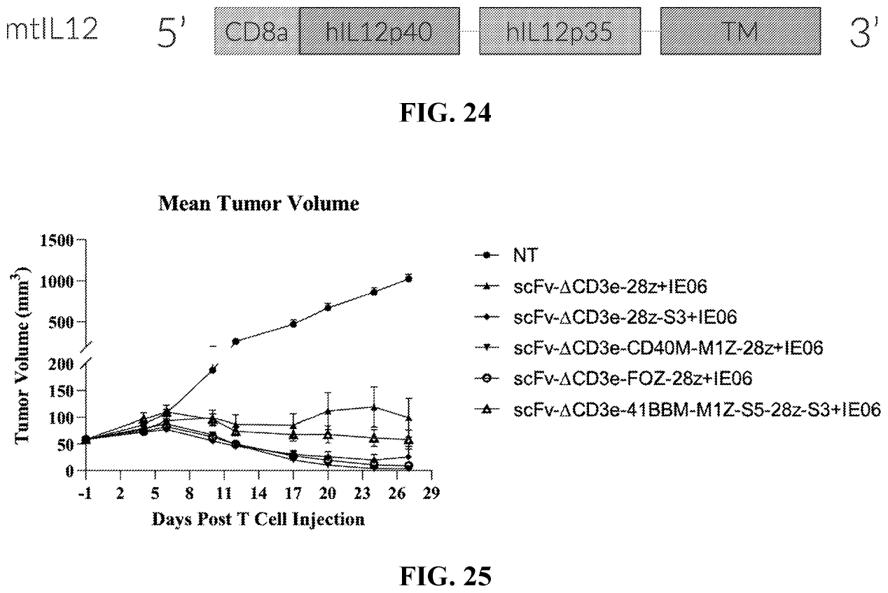

In vivo antitumor efficacy and proliferation capability of Aspire-T cells were further assessed using NSG (NOD scid gamma) mouse model. In these characterization experiments, the following constructs were used to transduce T cells to thereby obtain Aspire-T cells: scFv-ΔCD3e-28z (Design B), scFv-ΔCD3e-FOZ-28z (Design D), scFv-ΔCD3e-CD40M-M1Z-28z (Design H), scFv-ΔCD3e-28z-S3 (Design I), and scFv-ΔCD3e-41BBM-M1Z-S5-28z-S3 (Design K). In each of these constructs, the scFv is anti-ALPP A02 scFv (SEQ ID NO: 30), and all other sequences (including the motif/region/domain sequences and linker sequences, etc.) are same as the above-mentioned counterpart constructs where the anti-ALPP F8 scFv (SEQ ID NO: 9) is used. In addition, each of the above Aspire-T cells were further armored with (i.e. co-expressing) a membrane-tethered IL12 (i.e. mtIL12), with the structure diagram (IE06) shown in FIG. 24 and sequence shown in SEQ ID NO: 31.

Briefly, 48 female NSG mice were subcutaneously implanted with 5.0×10⁶ target tumor cells in 100 μl PBS. 28 days post tumor cell implantation, on Day-1, the animals were sorted into groups based on tumor size, with tumors in each group having an approximately equal average size of about 58 mm³. Then, the mice were intravenously injected with 2.5×10⁶ CAR+ T cells (i.e. T cells co-expressing each Aspire-TCR construct and mtIL12 IE06 as described above), or an equivalent number of non-transduced (“NT”) cells (8.3× 10⁶/mouse) as indicated. Caliper measurements were recorded twice a week for all animals during the study. The tumor volume was calculated using the formula: width²×length/2.

As shown in FIG. 25, in mouse studies, when treated with same dosage of T cells, all the armored Aspire-T cells showed inhibition of tumor growth, when compared to the control “NT” T cells. More specifically, the IE06-armored Aspire-T cells expressing scFv-ΔCD3e-28z-S3 (Design I), scFv-ΔCD3e-CD40M-M1Z-28z (Design H), or scFv-ΔCD3e-FOZ-28z (Design D) showed the highest antitumor activity, followed by the IE06-armored Aspire-T cells expressing scFv-ΔCD3e-41BBM-M1Z-S5-28z-S3 (Design K); they all exhibited a higher antitumor activity than the IE06-armored Aspire-T cells expressing scFv-ΔCD3e-28z (Design B) in terms of the in vivo antitumor efficacy. It is noted that even the IE06-armored Aspire-T cells expressing scFv-ΔCD3e-28z (Design B) still showed great antitumor efficacy even after about 1 month.

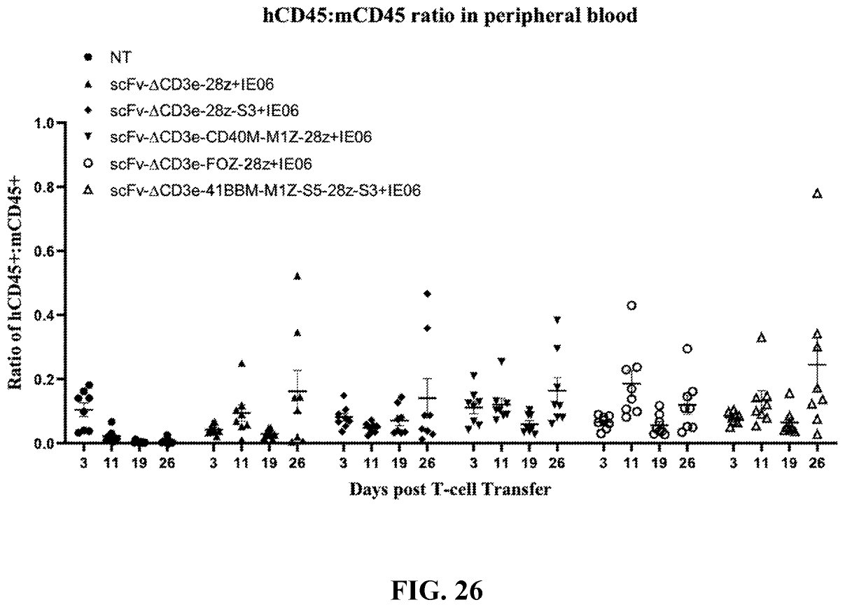

In the same mouse experiment, post T cell injection, blood samples were collected on the specified days (Day 3, Day 11, Day 19 and Day 26), and the hCD45: mCD45 ratio in peripheral blood was analyzed by FACS. As shown in FIG. 26, the peripheral blood analysis showed that the IE06-armored Aspire-T cells expressing scFv-ΔCD3e-CD40M-M1Z-28z (Design H) or scFv-ΔCD3e-FOZ-28z (Design D) showed the highest cell expansion and persistence, and the IE06-armored Aspire-T cells expressing scFv-ΔCD3e-28z-S3 (Design I) and scFv-ΔCD3e-41BBM-M1Z-S5-28z-S3 (Design K) showed similar T cell expansion, compared to IE06-armored CAR+ T cells expressing scFv-ΔCD3e-28z (Design B).

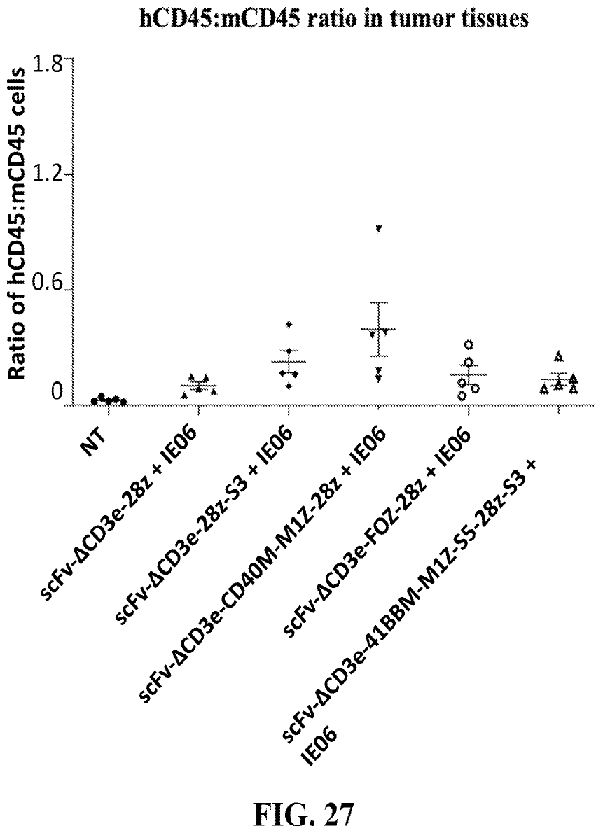

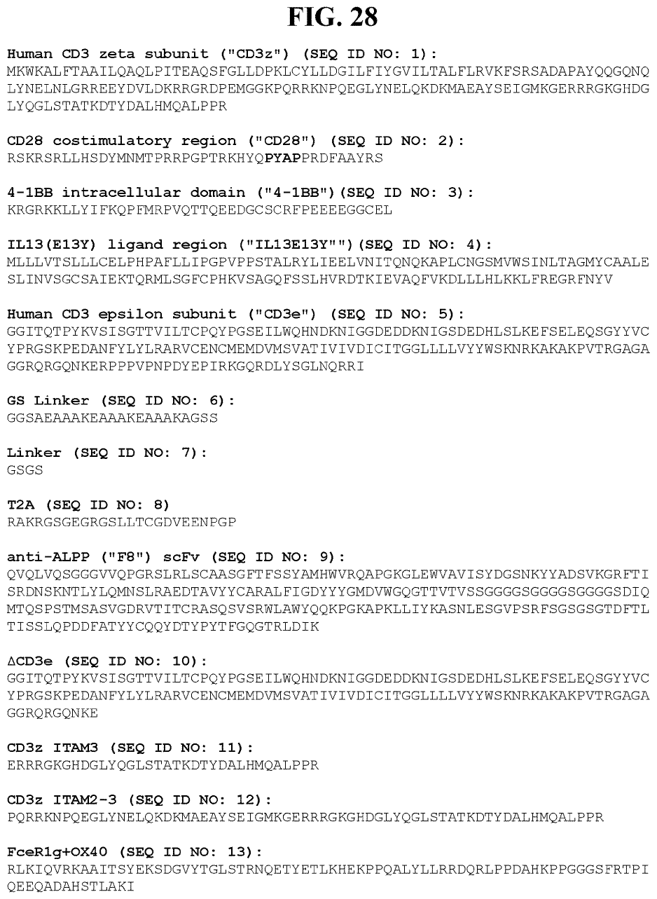

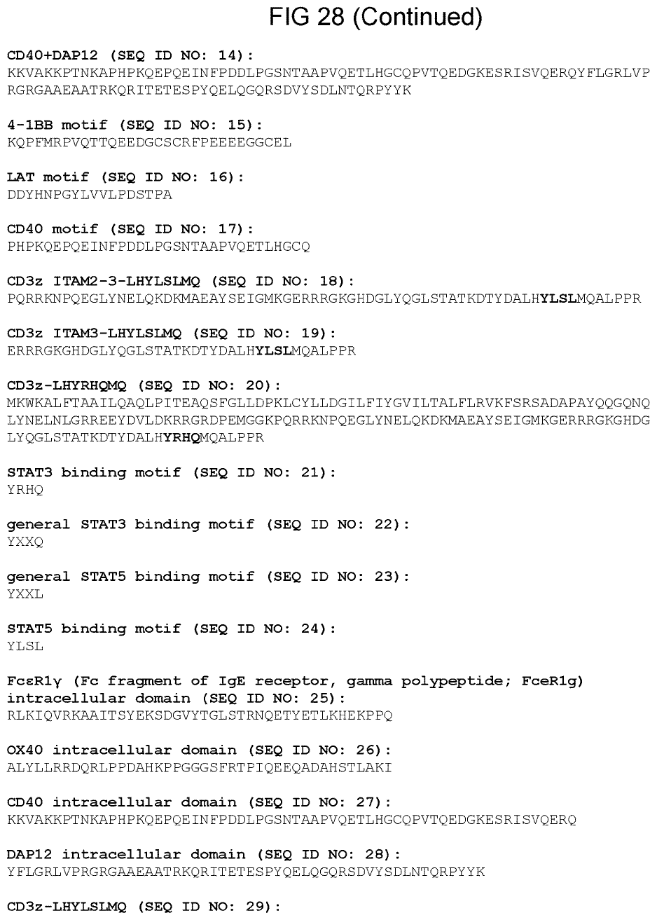

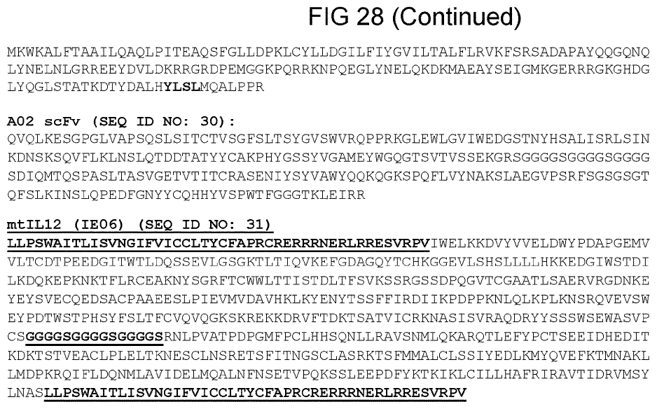

Additionally, on Day 27 post T cell injection, all remaining mice were euthanized and individual tumors were removed from each mouse. The tumors were digested, followed by passing through a 70 μm filter. The resulting lysate was collected and processed for staining and subsequent FACS analysis. As shown in FIG. 27, the analysis on tumor infiltrated T cells revealed that all IE06-armored Aspire-T cells had a higher T cell population in the tumor tissues than the control “NT” T cells. Additionally, compared to the IE06-armored T cells expressing scFv-ΔCD3e-28z (Design B), the IE06-armored T cells expressing scFv-ΔCD3e-FOZ-28z (Design D) and scFv-ΔCD3e-41BBM-M1Z-S5-28z-S3 (Design K) showed a similar hCD45: mCD45 ratio in tumor tissues, whereas the IE06-armored T cells expressing scFv-ΔCD3e-28z-S3 (Design I) and scFv-ΔCD3e-CD40M-M1Z-28z (Design H) showed a higher hCD45:mCD45 ratio than the armored T cells expressing scFv-ΔCD3e-28z (Design B), with the armored T cells expressing scFv-ΔCD3e-CD40M-M1Z-28z (Design H) notably exhibited the highest hCD45:mCD45 ratio.

4.7. Summary of Data from the Aspire-TCR Constructs

Based on the data above, the following observations were made.

Design A (scFv-CD3e-287): it was mainly used as a reference to evaluate other designs.

Design B (scFv-ΔCD3e-28z): On top of Design A, the ICD (intracellular domain) of CD3 epsilon (CD3e) was removed. The experimental results demonstrated that it had a better cell surface expression than Design A. Surprisingly, without the intracellular signaling domain, in Design B, a similar cytolytic activity and T cell activation against tumor targets were observed, while more central memory T cells (Tcm) were shown in Design B, as compared to Design A. Upon repeated antigen stimulation, Design B showed significant better T cell expansion than Design A, indicating that Design B is more memory like and has better persistency.

Design C (scFv-ΔCD3eZ-28z): On top of Design B, CD3 zeta (CD3z) ITAM 2&3 was added to the truncated CD3e to provide stronger downstream signaling (two ITAMs as compared to a single ITAM). Surprisingly, the experimental results revealed that Design C showed a very similar T cell activity, in vitro expansion, and T cell proliferation upon repeated antigen stimulation, whereas it showed a higher percentage of Tcm, compared to Design B and Design A, indicating that ITAM 2&3 of CD3z can drive different T cell phenotypes from CD3e ITAM.

Design D (scFv-ΔCD3e-FOZ-28z): First, OX40 has been previously reported to enhance CAR-T cell proliferation and persistence while reducing exhaustion (Sci. Transl. Med. 13, eaba7308 (2021); Int. J. Cancer 129, 2935-2944 (2011)). Second, the use of CD3z ITAM3 in lieu of its full-length counterpart has been shown to elicit preferential differentiation of long-lived central memory subsets in a CD28-based second-generation CAR (Nat. Med. 25, 82-88 (2019)). Third, FcεR1γ is a protein that is encoded by the FCER1G gene. It is a subunit of the high-affinity IgE receptor, which is expressed on mast cells and basophils. There is some evidence to suggest that FcεR1γ signaling may play a role in CAR-T cell function. One study found that CAR-T cells expressing a chimeric receptor that incorporated the FcεR1γ signaling domain exhibited enhanced cytokine production and cytotoxicity against cancer cells (J Transl Med 21, 197 (2023)). Therefore, in Design D, the combination of co-stimulatory signaling of FcεR1γ and OX40 and the signaling domain of CD3z ITAM3 were added to the ICD of CD3e, to achieve an enhanced and more persistent T cell activity. The experimental data revealed that compared to Design B, Design D showed a similar T cell cytolytic activity and activation upon antigen stimulation. Design D also showed similar in vitro expansion and memory phenotyping.

Design E (scFv-ΔCD3e-40DZ-287): The CD40 intracellular domains can provide co-stimulation of CAR-T cells to activate NF-κB and the subsequent expression of T cell co-stimulatory molecules in a manner that is discrete from 4-1BB signaling (J. Exp. Med. 2003; 198:1023-34). Therefore, in Design E, the co-stimulatory signaling of CD40 and DAP12 and signaling domain of CD3z ITAM3 were added, to achieve an enhanced and more persistent T cell activity. The experimental data revealed that compared to Design B, Design E had a similar T cell cytolytic activity and activation upon antigen stimulation. It had similar memory phenotyping, while for in vitro expansion, Design E showed a slower proliferation rate than Design B.

Design F (scFv-ΔCD3e-41BBMZ-28z): 41BB is a critical co-stimulatory factor in T cell development and activation, and upon engagement with its ligand (CD137L), can enhance T-cell survival, proliferation, and cytokine production. T cells expressing anti-CD19 CARs containing CD137 exhibited the greatest antileukemic efficacy and prolonged (>6 months) survival (Mol Ther. 2009 August; 17 (8): 1453-64). In this design, the 41BB ICD and CD3z ITAM3 were added to the truncated CD3e, aiming to provide an additional co-stimulatory signal to the T cells, in addition to CD28z. The experimental data revealed that compared to Design B, Design F had a better T cell cytolytic activity and activation upon antigen stimulation, which may be related to more Teff and less Tcm. For in vitro expansion, Design F showed slightly lower expression and slower proliferation rate than Design B. In comparison with Design E, Design F showed more Teff, less Tcm and a slightly better cytolytic activity and T cell activation, which may indicate that CD40 co-stimulatory signaling can drive the T cells towards to be more memory like than 4-1BB.

Design G (scFv-ΔCD3e-41BBM-M1Z-28z): To improve the viral transduction efficiency and increase the transgene expression, functional motif instead of domains (e.g., 4-1BB motif instead of 4-1BB intracellular domain) were used. In addition, the Linker for activation of T cells (LAT) is a pivotal protein that becomes tyrosine-phosphorylated swiftly upon TCR pathway activation. This post-translational modification transforms LAT into a critical docking platform for proteins containing SH2 domains. The phosphorylation of LAT facilitates the assembly of numerous adaptor proteins and signaling molecules into complex multimolecular signaling structures. These complexes can be strategically positioned in proximity to the TCR engagement site, thereby orchestrating downstream signaling events that are essential for T cell activation. In the current design, LAT motif was added to preposition the downstream signaling and enhance the T cell activity. The experimental data showed that Design G had a bit higher expression than Design B. In memory phenotyping analysis, Design G revealed much more Tcm than Design B in culture, which may be due to the cooperation of both 4-1BB co-stimulatory and LAT signaling. Function validation showed that Design G had a similar cytolytic activity against tumor targets and slightly better expansion upon repeated Antigen stimulation, which is in consistent with the higher percentage of Tcm and indicates a better persistence.

Design H (scFv-ΔCD3e-CD40M-M1Z-28z): In this design, different from Design G, CD40, instead of 4-1BB, was used to drive a more stem/memory like T cell phenotyping and better T cell persistence. Similarly, to minimize the vector size, instead of using the entire ICD, the CD40 motif was used. Experimental validation showed that similar to Design G, Design H had a higher viral transduction efficiency and better expression on primary T cells than Design B. It also showed significantly more Tom and Tn (naïve T cells) than Design B in culture, which may explain why Design H had slower proliferation, lower cytolytic activities, and T cell activation against tumor targets. Compared to Design G, Design H had slightly higher Tn. In the in vitro antigen repetitive stimulation, Design H showed similar T cell expansion. However, in the animal studies, Design H demonstrated significantly better T cell in vivo proliferation and persistence, as well as good antitumor efficacy.

In summary of the above for Designs A-H, the CD3zeta (CD3z) ITAM motif significantly influenced T cell phenotypes, promoting a more memory-like state compared to the CD3epsilon (CD3e) ITAM; the CD40 co-stimulatory signaling can skew T cells towards a memory-like phenotype more effectively than 4-1BB signaling; utilizing a smaller motif can reduce the overall vector size, which has been shown to enhance transgene expression in primary T cells; and the strategic incorporation of co-stimulatory signaling elements, specifically CD40 or 4-1BB, along with LAT, can markedly boost T cell “stemness”, leading to improved T cell persistence. These insights paved the way for optimizing T cell-based therapies and may significantly impact the development of more effective and lasting immunotherapies.

The optimal activation and proliferation of T cells necessitate a triad of signals: TCR engagement (signal 1), co-stimulation (signal 2), and cytokine engagement (signal 3). Presently, the CAR constructs under clinical evaluation encompass both a CD3z domain for TCR signaling and a domain for co-stimulation. However, they lack a domain for transmitting crucial signal 3. Signal 3 is particularly significant as it involves cytokines that interact with the common γ chain receptors, pivotal for T cell immunity, primarily via the JAK-STAT signaling pathway. The cytokines IL-2, IL-7, and IL-15 primarily activate STAT5 through its association motif YXXL (SEQ ID NO: 23), whereas, IL-21 distinctively activates STAT3 through its association motif YXXQ (SEQ ID NO: 22) within the IL-21 receptor.

In the following designs, the STAT motifs were added into the Aspire-TCR constructs to enable Aspire-T cells to induce cytokine signaling after antigen stimulation therefore to achieve better T cell activity.

Design I (scFv-ΔCD3e-28z-S3): On top of Design B, STAT3 binding motif (YRHQ) was incorporated into the expressed CD3z. Compared to Design B, Design I had a slightly better expression of Aspire on primary T cells and more Teff in culture. Additionally, in the in vitro antigen repetitive stimulation, better T cell expansion was observed. Consistently, in the animal studies, Design I also demonstrated better in vivo proliferation after T cell transfer, a higher T cell population in tumor tissues and a more significant antitumor activity, indicating that the addition of STAT3 signaling enhanced T cell proliferation and likely T cell persistence as well.

Design J (scFv-ΔCD3e-S5-28z-S3): On top of Design B, with the deletion of ICD (intracellular domain) of CD3 epsilon (CD3e), CD3 zeta (CD3z) ITAM 2&3 was added to the truncated CD3e to potentially provide stronger downstream signaling (two ITAMs as compared to a single ITAM). The STAT5 binding motif (YLSL) was incorporated into the CD3z fused to CD3e, and STAT3 binding motif (YRHQ) to the separately expressed CD3z in order to provide both STAT5 and STAT3 cytokine downstream signaling, aiming for a better T cell activity and long-term persistence. Surprisingly, a better T cell activity in Design J similar to Design I was not observed. The experimental results showed that Design J had a similar expression level comparing to Design B. Also, the in vitro functionality (cytotoxicity and T cell activation) had a little difference from Design B. Upon antigen repetitive stimulation, it had similar T cell expansion. It may suggest that the combination of CD28 co-stimulation and both STAT3 & STAT5 signaling would not provide the optimal T cell activation and proliferation.

Design K (scFv-ΔCD3e-41BBM-M1Z-S5-28z-S3): On top of Design J, 41BB-LAT motifs were incorporated into the design to further improve T cell activation and proliferation. Design K had more Tn and Teff cells, compared to either Design B or Design J. It also had better T cell expansion with or without repeated antigen stimulation. Functionality validation demonstrated that Design K had a stronger cytolytic activity and higher cytokine production upon antigen stimulation. On the other hand, Design K did not show a significant antitumor activity, which may be due to the fact that IE06 (IL12) armor is too strong and led the Design K cells to be easier exhausted.

In summary of Designs I-K, the integration of cytokine signaling pathways, particularly those mediated by STAT3 and/or STAT5, has been found to amplify T cell proliferation, which is also indicative of enhanced T cell persistence; and the strategic selection of co-stimulatory signals in combination with cytokine signaling pathways is crucial. Specifically, the synergy of 4-1BB (and potentially CD40) with STAT3 and/or STAT5 signaling markedly improves T cell activation and proliferation. This enhancement is poised to boost the potential antitumor efficacy of T cell-based therapies. These conclusions underscore the importance of fine-tuning T cell therapies by manipulating both co-stimulatory and cytokine signaling to maximize therapeutic outcomes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered CD3 zeta (CD3z) subunit, comprising a co-stimulatory region operably linked to or incorporated into a CD3 zeta component, wherein:
the CD3 zeta component comprises a human CD3 zeta or a functional portion or a functional variant thereof, comprising a sequence that has at least 80% sequence identity to SEQ ID NO: 1; and
the co-stimulatory region is within an intracellular domain of the engineered CD3 zeta subunit.

2. The engineered CD3 zeta subunit of claim 1, wherein when expressed in an immune cell, at least one of the following is met:
(1) the immune cell expressing the engineered CD3 zeta subunit has a reduced activation in the absence of antigen stimulation compared to when the immune cell does not express the engineered CD3 zeta subunit;
(2) the immune cell expressing the engineered CD3 zeta subunit has a reduced cytotoxicity against non-target cells compared to when the immune cell does not express the engineered CD3 zeta subunit;
(3) the immune cell expressing the engineered CD3 zeta subunit has an increased activation upon antigen stimulation compared to when the immune cell does not express the engineered CD3 zeta subunit; and
(4) the immune cell expressing the engineered CD3 zeta subunit has an increased immune cell response against target cells corresponding thereto compared to when the immune cell does not express the engineered CD3 zeta subunit.

3. The engineered CD3 zeta subunit of claim 1 or claim 2, wherein the CD3 zeta component comprises an amino acid sequence as set forth in SEQ ID NO: 1.

4. The engineered CD3 zeta subunit of any one of claims 1-3, wherein the co-stimulatory region comprises a co-stimulatory domain, or a functional portion or a functional variant thereof, of a protein selected from the group consisting of CD28, 4-1BB, OX40, CD2, CD27, CDS, ICAM-1, LFA-1, and ICOS.

5. The engineered CD3 zeta subunit of claim 4, wherein the co-stimulatory region comprises a co-stimulatory domain, or a functional portion or a functional variant thereof, of CD28, comprising a sequence that has at least 80% sequence identity to SEQ ID NO: 2.

6. The engineered CD3 zeta subunit of claim 4, wherein the co-stimulatory region comprises a co-stimulatory domain, or a functional portion or a functional variant thereof, of 4-1BB, comprising a sequence that has at least 80% sequence identity to SEQ ID NO: 3.

7. The engineered CD3 zeta subunit of any one of claims 1-6, wherein the co-stimulatory region is operably fused to the C-terminus of the CD3 zeta component.

8. The engineered CD3 zeta subunit of any one of claims 1-6, wherein the co-stimulatory region is located between a transmembrane domain and an intracellular domain of the CD3 zeta component.

9. The engineered CD3 zeta subunit of any one of claims 1-8, further comprising a STAT binding region within an intracellular domain thereof.

10. The engineered CD3 zeta subunit of claim 9, wherein the STAT binding region comprises a STAT3 binding motif that comprises an amino acid sequence that is at least 50%, 75%, or 100% identical to the amino acid sequence of SEQ ID NO: 22.

11. The engineered CD3 zeta subunit of claim 10, wherein the STAT3 binding motif comprises an amino acid sequence that is at least 50%, 75%, or 100% identical to the amino acid sequence of SEQ ID NO: 21.

12. The engineered CD3 zeta subunit of claim 9, wherein the STAT binding region comprises a STAT5 binding motif that comprises an amino acid sequence that is at least 50%, 75%, or 100% identical to the amino acid sequence of SEQ ID NO: 23.

13. The engineered CD3 zeta subunit of claim 12, wherein the STAT5 binding motif comprises an amino acid sequence that is at least 50%, 75%, or 100% identical to the amino acid sequence of SEQ ID NO: 24.

14. The engineered CD3 zeta subunit of any one of claims 9-13, wherein the STAT binding region is located within a region of the CD3 zeta component corresponding to positions 150-164 of SEQ ID NO: 1.

15. The engineered CD3 zeta subunit of claim 14, wherein the STAT binding region is an insertion at a location of the CD3 zeta component corresponding to between position 157 and position 158 of SEQ ID NO: 1.

16. The engineered CD3 zeta subunit of claim 15, wherein the CD3 zeta component comprises an amino acid sequence that is at least 80%, 85%, 90%, 95%, or 100% identical to the amino acid sequence of SEQ ID NO: 20.

17. The engineered CD3 zeta subunit of claim 15, wherein the CD3 zeta component comprises an amino acid sequence that is at least 80%, 85%, 90%, 95%, or 100% identical to the amino acid sequence of SEQ ID NO: 29.

18. The engineered CD3 zeta subunit of any one of claims 9-17, wherein when the engineered CD3 zeta subunit is expressed in a population of T cells, at least one of the following is met if compared to when the STAT binding region is absent in the engineered CD3 zeta subunit:
the population of T cells exhibit an increased expansion in vitro upon repeated stimulation with the antigen;
the population of T cells exhibit an increased efficacy in vivo against tumors that comprise the target cells; and
the population of T cells exhibit a higher ratio in tumor tissues infiltrated therewith in vivo.

19. The engineered CD3 zeta subunit of any one of claims 1-18, wherein the immune cell is a T lymphocyte, a tumor-infiltrating lymphocyte (TIL), or a natural kill (NK) cell.

20. The engineered CD3 zeta subunit of any one of claims 1-19, wherein when the engineered CD3 zeta subunit is expressed in a population of T cells expressing a T cell receptor (TCR), the population of T cells exhibit an increased surface expression compared to when the co-stimulatory region is absent in the engineered CD3 zeta subunit.

21. A method for modulating activities of an immune cell, comprising:
expressing in the immune cell the engineered CD3 zeta subunit according to any one of claims 1-20.

22. The method of claim 21, wherein the immune cell expressing the engineered CD3 zeta subunit:
(1) has a reduced activation in the absence of antigen stimulation compared to when the immune cell does not express the engineered CD3 zeta subunit;
(2) has a reduced cytotoxicity against non-target cells compared to when the immune cell does not express the engineered CD3 zeta subunit;
(3) has an increased activation upon antigen stimulation compared to when the immune cell does not express the engineered CD3 zeta subunit; or
(4) has an increased immune cell response against target cells corresponding thereto compared to when the immune cell does not express the engineered CD3 zeta subunit.

23. An engineered CD3 epsilon (CD3e) subunit, comprising a target-recognizing region and a CD3 epsilon component, wherein:
the target-recognizing region is operably fused to an extracellular domain of the engineered CD3 epsilon subunit; and
the CD3 epsilon component comprises a truncated CD3 epsilon with a truncation at a region of a human CD3 epsilon subunit corresponding to the intercellular domain thereof.

24. The engineered CD3 epsilon subunit of claim 23, wherein the truncation is within a region of the human CD3 epsilon subunit corresponding to positions 151-179 of SEQ ID NO: 5.

25. The engineered CD3 epsilon subunit of claim 24, wherein the CD3 epsilon component comprises an amino acid sequence that is at least 80%, 85%, 90%, 95%, or 100% identical to the amino acid sequence of SEQ ID NO: 10.

26. The engineered CD3 epsilon subunit of claim 25, wherein the CD3 epsilon component comprises the amino acid sequence of SEQ ID NO: 10.

27. The engineered CD3 epsilon subunit of any one of claims 23-26, wherein when the engineered CD3 epsilon subunit is expressed in a population of T cells, the population of T cells exhibit an increased surface expression of the engineered CD3 epsilon subunit and/or an increased expansion when compared to when the CD3 epsilon component comprises a full-length CD3 epsilon subunit.

28. The engineered CD3 epsilon subunit of any one of claims 23-26, wherein the C-terminus of the CD3 epsilon component is the C-terminus of the engineered CD3 epsilon subunit.

29. The engineered CD3 epsilon subunit of claim 28, wherein when the engineered CD3 epsilon subunit is expressed in a population of T cells, at least one of the following is met:
(1) the population of T cells exhibit an increased surface expression of the engineered CD3 epsilon subunit, comparing to when the CD3 epsilon component comprises a full-length CD3 epsilon subunit;
(2) the population of T cells comprise a higher percentage of central memory T cells, comparing to when the CD3 epsilon component comprises a full-length CD3 epsilon subunit; and
(3) the population of T cells exhibit an increased expansion in vitro upon repeated stimulation with the antigen, comparing to when the CD3 epsilon component comprises a full-length CD3 epsilon subunit.

30. The engineered CD3 epsilon subunit of any one of claims 23-26, further comprising at least one functional region in an intracellular domain thereof, wherein the at least one functional region is operably fused to the C-terminus of the CD3 epsilon component.

31. The engineered CD3 epsilon subunit of claim 30, wherein the at least one functional region comprises an immune receptor tyrosine based activation motif (ITAM).

32. The engineered CD3 epsilon subunit of claim 31, wherein the ITAM is derived from CD3z, CD3g, DAP12, FcαRI, FcγRI, FcγRII, FcγRIII, Dectin-1, CLEC-1, CD28, or CD72.

33. The engineered CD3 epsilon subunit of claim 32, wherein the ITAM comprises:
(1) a CD3z ITAM3 that comprises an amino acid sequence that is at least 80%, 85%, 90%, 95%, or 100% identical to the amino acid sequence of SEQ ID NO: 11; or
(2) a CD3z ITAM2-3 that comprises an amino acid sequence that is at least 80%, 85%, 90%, 95%, or 100% identical to the amino acid sequence of SEQ ID NO: 12.

34. The engineered CD3 epsilon subunit of any one of claims 31-33, wherein the ITAM is located at the C-terminus of the engineered CD3 epsilon subunit.

35. The engineered CD3 epsilon subunit of claim 34, wherein the ITAM is operably fused to the C-terminus of the CD3 epsilon component, wherein the ITAM comprises a CD3z ITAM3 or a CD3z ITAM2-3.

36. The engineered CD3 epsilon subunit of claim 35, wherein the ITAM comprises a STAT binding region, wherein the STAT binding region is a STAT3 binding motif or a STAT5 binding motif.

37. The engineered CD3 epsilon subunit of any one of claims 31-36, wherein when the engineered CD3 epsilon subunit is expressed in a population of T cells, the population of T cells comprise a higher percentage of central memory T cells compared to when the ITAM is absent in the engineered CD3 epsilon subunit.

38. The engineered CD3 epsilon subunit of any one of claims 30-37, wherein the at least one functional region comprises any one or a combination of:
(1) an FcεR1γ intracellular domain, which comprises an amino acid sequence that is at least 80%, 85%, 90%, 95%, or 100% identical to the amino acid sequence of SEQ ID NO: 25;
(2) an OX40 intracellular domain, which comprises an amino acid sequence that is at least 80%, 85%, 90%, 95%, or 100% identical to the amino acid sequence of SEQ ID NO: 26:
(3) a CD40 intracellular domain, which comprises an amino acid sequence that is at least 80%, 85%, 90%, 95%, or 100% identical to the amino acid sequence of SEQ ID NO: 27;
(4) a DAP12 intracellular domain, which comprises an amino acid sequence that is at least 80%, 85%, 90%, 95%, or 100% identical to the amino acid sequence of SEQ ID NO: 28:
(5) a 4-1BB intracellular domain, which comprises an amino acid sequence that is at least 80%, 85%, 90%, 95%, or 100% identical to the amino acid sequence of SEQ ID NO: 3;
(6) a 4-1BB motif, which comprises an amino acid sequence that is at least 80%, 85%, 90%, 95%, or 100% identical to the amino acid sequence of SEQ ID NO: 15;
(7) a CD40 motif, which comprises an amino acid sequence that is at least 80%, 85%, 90%, 95%, or 100% identical to the amino acid sequence of SEQ ID NO: 17;
(8) a linker for activation of T cells (LAT) motif, which comprises an amino acid sequence that is at least 80%, 85%, 90%, 95%, or 100% identical to the amino acid sequence of SEQ ID NO: 16; and
(9) a CD28 intracellular domain, which comprises an amino acid sequence that is at least 80%, 85%, 90%, 95%, or 100% identical to the amino acid sequence of SEQ ID NO: 2.

39. The engineered CD3 epsilon subunit of any one of claims 23-38, further comprising a STAT binding region within an intracellular domain thereof.

40. The engineered CD3 epsilon subunit of claim 39, wherein the STAT binding region comprises:
(1) a STAT3 binding motif, which comprises an amino acid sequence that is at least 50%, 75%, or 100% identical to the amino acid sequence of SEQ ID NO: 22; or
(2) a STAT5 binding motif, which comprises an amino acid sequence that is at least 50%, 75%, or 100% identical to the amino acid sequence of SEQ ID NO: 23.

41. The engineered CD3 epsilon subunit of claim 40, wherein the STAT binding region comprises a STAT3 binding motif, which comprises an amino acid sequence that is at least 50%, 75%, or 100% identical to the amino acid sequence of SEQ ID NO: 21.

42. The engineered CD3 epsilon subunit of claim 40, wherein the STAT binding region comprises a STAT5 binding motif, which comprises an amino acid sequence that is at least 50%, 75%, or 100% identical to the amino acid sequence of SEQ ID NO: 24.

43. The engineered CD3 epsilon subunit of any one of claims 39-42, further comprising an ITAM in the intracellular domain thereof, wherein ITAM comprises the STAT binding region.

44. The engineered CD3 epsilon subunit of claim 43, wherein the ITAM is a CD3z ITAM2-3 motif, which comprises an amino acid sequence that is at least 80%, 85%, 90%, 95%, or 100% identical to the amino acid sequence of SEQ ID NO: 18.

45. The engineered CD3 epsilon subunit of claim 43, wherein the ITAM is a CD3z ITAM3 motif, which comprises an amino acid sequence that is at least 80%, 85%, 90%, 95%, or 100% identical to the amino acid sequence of SEQ ID NO: 19.

46. The engineered CD3 epsilon subunit of claim 38, wherein the at least one functional region comprises both an FcεR1γ intracellular domain and an OX40 intracellular domain.

47. The engineered CD3 epsilon subunit of claim 46, wherein the at least one functional region comprises a first compound functional region comprising, from an N-terminus to a C-terminus direction, the FcεR1γ intracellular domain and the OX40 intracellular domain, wherein the first compound functional region comprises an amino acid sequence that is at least 80%, 85%, 90%, 95%, or 100% identical to the amino acid sequence of SEQ ID NO: 13.

48. The engineered CD3 epsilon subunit of claim 46 or 47, wherein the at least one functional region further comprises, over the C-terminus of the first compound functional region, an ITAM, wherein the ITAM comprises CD3z ITAM3 or CD3z ITAM2-3.

49. The engineered CD3 zeta subunit of claim 48, wherein when the engineered CD3 zeta subunit is expressed in a population of T cells, the population of T cells exhibit an increased efficacy in vivo against tumors that comprise the target cells compared to when the FcεR1γ intracellular domain, the OX40 intracellular domain, and the ITAM are all absent in the engineered CD3 zeta subunit.

50. The engineered CD3 epsilon subunit of claim 38, wherein the at least one functional region comprises both a CD40 intracellular domain and a DAP12 intracellular domain.

51. The engineered CD3 epsilon subunit of claim 50, wherein the at least one functional region comprises a second compound functional region comprising, from an N-terminus to a C-terminus direction, the CD40 intracellular domain and the DAP12 intracellular domain, wherein the second compound functional region comprises an amino acid sequence that is at least 80%, 85%, 90%, 95%, or 100% identical to the amino acid sequence of SEQ ID NO: 14.

52. The engineered CD3 epsilon subunit of claim 50 or claim 51, wherein the at least one functional region further comprises, at the C-terminus of the second compound functional region, an ITAM, wherein the ITAM comprises a CD3z ITAM3 or a CD3z ITAM2-3.

53. The engineered CD3 epsilon subunit of any one of claims 23-52, further comprising, from the N-terminus to the C-terminus thereof, a 4-1BB intracellular domain and an ITAM, wherein the ITAM comprises a CD3z ITAM3 or a CD3z ITAM2-3.

54. The engineered CD3 epsilon subunit of claim 53, wherein when the engineered CD3 epsilon subunit is expressed in a T cell that is specifically against a target cell having a specific antigen, at least one of the following is met if compared to when the 4-1BB intracellular domain and the ITAM are absent in the engineered CD3 epsilon subunit:
the T cell exhibits an increased cytotoxicity against the target cell in vitro; and
the T cell exhibits an increased stimulation by the antigen in vitro.

55. The engineered CD3 epsilon subunit of claim 38, wherein the at least one functional region comprises both a 4-1BB motif and an LAT motif.

56. The engineered CD3 epsilon subunit of claim 55, further comprising an ITAM at the C-terminus of the engineered CD3 epsilon subunit, wherein the ITAM comprises a CD3z ITAM3 or a CD3z ITAM2-3.

57. The engineered CD3 epsilon subunit of claim 56, wherein when the engineered CD3 epsilon subunit is expressed in a population of T cells, at least one of the following is met if compared to when the 4-1BB motif, the LAT motif and the ITAM are all absent in the engineered CD3 epsilon subunit:
the population of T cells comprise a higher percentage of central memory T cells; and
the population of T cells exhibit an increased expansion in vitro upon repeated stimulation with the antigen.

58. The engineered CD3 epsilon subunit of claim 56, wherein the ITAM comprises a STAT binding region, wherein the STAT binding region is a STAT3 binding motif or a STAT5 binding motif.

59. The engineered CD3 epsilon subunit of claim 38, wherein the at least one functional region comprises both a CD40 motif and an LAT motif.

60. The engineered CD3 epsilon subunit of claim 59, further comprising an ITAM at the C-terminus of the engineered CD3 epsilon subunit, wherein the ITAM comprises a CD3z ITAM3 or a CD3z ITAM2-3.

61. The engineered CD3 zeta subunit of claim 60, wherein when the engineered CD3 zeta subunit is expressed in a population of T cells, at least one of the following is met if compared to when the CD40 motif, the LAT motif, and the ITAM are all absent in the engineered CD3 zeta subunit:
the population of T cells exhibit an increased surface expression of the engineered CD3 epsilon subunit;
the population of T cells comprise a higher percentage of naïve T cells;
the population of T cells comprise a higher percentage of central memory T cells;
the population of T cells exhibit an increased efficacy in vivo against tumors that comprise the target cells; and
the population of T cells exhibit a higher ratio in tumor tissues infiltrated therewith in vivo.

62. The engineered CD3 epsilon subunit of any one of claims 23-61, wherein the target-recognizing region is operably incorporated into the CD3 epsilon component.

63. The engineered CD3 epsilon subunit of any one of claims 23-61, wherein the target-recognizing region comprises:
(1) an antigen-binding region; or
(2) a ligand or a fragment thereof that binds to a cell-surface receptor expressed on a target cell of the immune cell.

64. The engineered CD3 epsilon subunit of claim 63, wherein the antigen-binding region comprises a single-chain variable fragment (scFv) that specifically recognizes ALPP, LYPD3, IL13Ra2, BCMA, CD16, CD19, CD20, CD22, CD27 CD138, CD33, CD123, CD171, CD70, CD7, CS-1, PSMA, PSCA, ROR1, GD2, MUC1, MUC16, HER2(ErbB2), MET, EphA2, EpCAM, CEA, CSPG4, Lewis Y antigen, Mesothelin, NKG2D, Glypican-3 (GPC-3), FAP, FRa (folate receptoralpha), EGFR, EGFR vIII, IL-11Ra (IL11 receptor alpha), VEGFR-II, B7-H6, and DNAM-1.

65. The engineered CD3 epsilon subunit of claim 64, wherein the antigen-binding region comprises a single-chain variable fragment (scFv) against ALPP.

66. The engineered CD3 epsilon subunit of claim 63, wherein the antigen-binding region comprises a single-domain antibody (sdAb or nanobody).

67. The engineered CD3 epsilon subunit of claim 63, wherein the ligand is selected from the group consisting of IL13 (E13Y), IL-13, IL-11, IL-10, APRIL, GM-CSF, TPO, Adnectin, T1E, FLT3L, EPHRIN B2, CTLX, LFA-1, and FSH.

68. The engineered CD3 epsilon subunit of claim 67, wherein the ligand is IL13 (E13Y).

69. The engineered CD3 epsilon subunit of any one claims 23-68, further comprising a flexible linker between the target-recognizing region and the CD3 epsilon component.

70. The engineered CD3 epsilon subunit of claim 69, wherein the flexible linker comprises a sequence of SEQ ID NO: 6.

71. An engineered TCR complex system, comprising at least one of:
the engineered CD3 zeta subunit according to any one of claims 1-20; and
the engineered CD3 epsilon subunit according to any one of claims 23-70.

72. The engineered TCR complex system of claim 71, comprising both the engineered CD3 epsilon subunit and the engineered CD3 zeta subunit.

73. The engineered TCR complex system of claim 72, wherein the co-stimulatory region of the engineered CD3 zeta subunit comprises:
a co-stimulatory domain, or a functional portion or a functional variant thereof, of CD28; or
a co-stimulatory domain, or a functional portion or a functional variant thereof, of 4-1BB.

74. The engineered TCR complex system of claim 72 or claim 73, wherein one or both of the engineered CD3 epsilon subunit and the engineered CD3 zeta subunit comprise a STAT binding region within an intracellular domain thereof.

75. The engineered TCR complex system of claim 74, wherein both of the engineered CD3 epsilon subunit and the engineered CD3 zeta subunit comprise a STAT binding region within an intracellular domain thereof, wherein the STAT binding region of the engineered CD3 epsilon subunit is different from the STAT binding region of the engineered CD3 zeta subunit.

76. The engineered TCR complex system of claim 75, wherein the STAT binding region of the engineered CD3 epsilon subunit and the STAT binding region of the engineered CD3 zeta subunit are (1) respectively a STAT3 binding motif and a STAT5 binding motif or (2) are respectively a STAT5 binding motif and a STAT3 binding motif.

77. The engineered TCR complex system of claim 74, wherein only the engineered CD3 zeta subunit comprises a STAT binding region within an intracellular domain thereof, wherein the STAT binding region comprises a STAT3 binding motif or a STAT5 binding motif.

78. The engineered TCR complex system of any one of claims 74-77, wherein when the engineered CD3 epsilon subunit is expressed in a population of T cells, at least one of the following is met if compared to when the STAT binding region is absent in any of the engineered CD3 epsilon subunit or the engineered CD3 zeta subunit:
the population of T cells comprise a higher percentage of terminally differentiated effector T cells;
the population of T cells exhibit an increased antigen-specific stimulation in vitro; and
the population of T cells exhibit an increased expansion in vitro upon repeated stimulation with the antigen.

79. The engineered TCR complex system of claim 71, comprising the engineered CD3 zeta subunit, and further comprising an engineered target-recognizing TCR subunit, wherein the TCR subunit is based on one of TCRα, TCRβ, TCRγ, TCRδ, CD3ε, CD3γ, or CD3δ, and preferably on one of TCRα, TCRβ, CD3ε, CD3γ, or CD3δ.

80. The engineered TCR complex system of claim 79, wherein the engineered target-recognizing TCR subunit comprises a target-recognizing region, the target-recognizing region comprising:
(1) an antigen-binding region, the antigen-binding region comprising at least one of a single-chain variable fragment (scFv) or a single-domain antibody; or
(2) a ligand or a fragment thereof that binds to a cell-surface receptor expressed on a target cell of the immune cell.

81. A chimeric polypeptide, comprising a target-recognizing region operably linked to or incorporated into one of a TCR alpha subunit, a TCR beta subunit, a CD3 gamma subunit, a CD3 delta subunit or a CD3 epsilon subunit, or a functional portion or a functional variant thereof, wherein:
the target-recognizing region comprises a ligand, a functional portion, or a functional variant thereof, wherein when the chimeric polypeptide is expressed in an immune cell, the target-recognizing region of the chimeric polypeptide is capable of binding to a cell-surface receptor expressed on a target cell of the immune cell.

82. The chimeric polypeptide of claim 81, wherein the target-recognizing region is operably linked to or incorporated into a CD3 epsilon subunit or a functional portion or a functional variant thereof.

83. The chimeric polypeptide of claim 82, wherein the CD3 epsilon subunit comprises an amino acid sequence that is at least 80%, 85%, 90%, 95%, or 100% identical to the amino acid sequence of SEQ ID NO: 5.

84. The chimeric polypeptide of claim 82, wherein the CD3 epsilon subunit comprises a truncated CD3 epsilon, which comprises an amino acid sequence that is at least 80%, 85%, 90%, 95%, or 100% identical to the amino acid sequence of SEQ ID NO: 10.

85. The chimeric polypeptide of any one of claims 81-84, wherein the ligand is selected from the group consisting of IL13 (E13Y), IL-13, IL-11, IL-10, APRIL, GM-CSF, TPO, Adnectin, T1E, FLT3L, EPHRIN B2, CTLX, LFA-1, and FSH.

86. The chimeric polypeptide of claim 85, wherein the ligand is IL13 (E13Y).

87. The chimeric polypeptide of claim 86, wherein the IL13 (E13Y) ligand comprises an amino acid sequence that is at least 80%, 85%, 90%, 95%, or 100% identical to the amino acid sequence of SEQ ID NO: 4.

88. An engineered immune cell, comprising:
the engineered TCR complex system according to any one of claims 71-80; and/or
the chimeric polypeptide according to any one of claims 79-85.

89. The engineered immune cell of claim 88, wherein the engineered immune cell is a T lymphocyte, a tumor infiltrating lymphocyte (TIL), or a natural killer (NK) cell.

90. A method for treating a subject in need thereof, comprising administrating to the subject a therapeutically effective amount of the engineered immune cell according to claim 88 or claim 89.


SEQUENCE LISTING (31 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 41 aa, synthetic construct
RSKRSRLLHSDYMNMTPRRPGPTRKHYQPYAPPRDFAAYRS

SEQ ID NO: 3 - protein, 42 aa, synthetic construct
KRGRKKLLYIFKQPFMRPVQTTQEEDGCSCRFPEEEEGGCEL

SEQ ID NO: 4 - protein, 136 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNYV

SEQ ID NO: 5 - protein, 179 aa, Homo sapiens
GGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 6 - protein, 23 aa, synthetic construct
GGSAEAAAKEAAAKEAAAKAGSS

SEQ ID NO: 7 - protein, 4 aa, synthetic construct
GSGS

SEQ ID NO: 8 - protein, 25 aa, synthetic construct
RAKRGSGEGRGSLLTCGDVEENPGP

SEQ ID NO: 9 - protein, 244 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARALFIGDYYYGMDVWGQGTTVTVSSGGGGSGGGGSGGGGSDIQMTQSPSTMSASVGDRVTITCRASQSVSRWLAWYQQKPGKAPKLLIYKASNLESGVPSRFSGSGSGTDFTLTISSLQPDDFATYYCQQYDTYPYTFGQGTRLDIK

SEQ ID NO: 10 - protein, 150 aa, synthetic construct
GGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKE

SEQ ID NO: 11 - protein, 34 aa, synthetic construct
ERRRGKGHDGLYQGLSTATKDTYDALHMQALPPR

SEQ ID NO: 12 - protein, 65 aa, synthetic construct
PQRRKNPQEGLYNELQKDKMAEAYSEIGMKGERRRGKGHDGLYQGLSTATKDTYDALHMQALPPR

SEQ ID NO: 13 - protein, 84 aa, synthetic construct
RLKIQVRKAAITSYEKSDGVYTGLSTRNQETYETLKHEKPPQALYLLRRDQRLPPDAHKPPGGGSFRTPIQEEQADAHSTLAKI

SEQ ID NO: 14 - protein, 114 aa, synthetic construct
KKVAKKPTNKAPHPKQEPQEINFPDDLPGSNTAAPVQETLHGCQPVTQEDGKESRISVQERQYFLGRLVPRGRGAAEAATRKQRITETESPYQELQGQRSDVYSDLNTQRPYYK

SEQ ID NO: 15 - protein, 31 aa, Synthetic construct
KQPFMRPVQTTQEEDGCSCRFPEEEEGGCEL

SEQ ID NO: 16 - protein, 18 aa, synthetic construct
DDYHNPGYLVVLPDSTPA

SEQ ID NO: 17 - protein, 33 aa, synthetic construct
PHPKQEPQEINFPDDLPGSNTAAPVQETLHGCQ

SEQ ID NO: 18 - protein, 69 aa, synthetic construct
PQRRKNPQEGLYNELQKDKMAEAYSEIGMKGERRRGKGHDGLYQGLSTATKDTYDALHYLSLMQALPPR

SEQ ID NO: 19 - protein, 38 aa, synthetic construct
ERRRGKGHDGLYQGLSTATKDTYDALHYLSLMQALPPR

SEQ ID NO: 20 - protein, 168 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHYRHQMQALPPR

SEQ ID NO: 21 - protein, 4 aa, synthetic construct
YRHQ

SEQ ID NO: 22-23 - skipped (2 entries)

SEQ ID NO: 24 - protein, 4 aa, synthetic construct
YLSL

SEQ ID NO: 25 - protein, 42 aa, synthetic construct
RLKIQVRKAAITSYEKSDGVYTGLSTRNQETYETLKHEKPPQ

SEQ ID NO: 26 - protein, 42 aa, synthetic construct
ALYLLRRDQRLPPDAHKPPGGGSFRTPIQEEQADAHSTLAKI

SEQ ID NO: 27 - protein, 62 aa, synthetic construct
KKVAKKPTNKAPHPKQEPQEINFPDDLPGSNTAAPVQETLHGCQPVTQEDGKESRISVQERQ

SEQ ID NO: 28 - protein, 52 aa, synthetic construct
YFLGRLVPRGRGAAEAATRKQRITETESPYQELQGQRSDVYSDLNTQRPYYK

SEQ ID NO: 29 - protein, 168 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHYLSLMQALPPR

SEQ ID NO: 30 - protein, 249 aa, synthetic construct
QVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIRR

SEQ ID NO: 31 - protein, 610 aa, synthetic construct
LLPSWAITLISVNGIFVICCLTYCFAPRCRERRRNERLRRESVRPVIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASLLPSWAITLISVNGIFVICCLTYCFAPRCRERRRNERLRRESVRPV